(19) United States
(12) Patent Application Publication
(10) Pub. No.: US 2024/0215443 A1
(43) Pub. Date: Jun. 27, 2024

(54) ORGANIC COMPOUND AND LIGHT-EMITTING DEVICE

(71) Applicant: SEMICONDUCTOR ENERGY LABORATORY CO., LTD., Atsugi-shi (JP)

(72) Inventors: Yui YOSHIYASU, Atsugi (JP); Nobuharu OHSAWA, Zama (JP); Hiromitsu KIDO, Atsugi (JP); Masatoshi TAKABATAKE, Atsugi (JP); Satoshi SEO, Sagamihara (JP)

(21) Appl. No.: 18/525,103

(22) Filed: Nov. 30, 2023

(30) Foreign Application Priority Data
Nov. 30, 2022 (JP) 2022-192372
Dec. 1, 2022 (JP) 2022-193138
Jan. 13, 2023 (JP) 2023-003459

(51) Int. Cl. H10K 85/60; C07D 209/86; C07F 7/08; C09K 11/06; H10K 85/40

(52) U.S. Cl. CPC H10K 85/6572; C07D 209/86; C07F 7/0812; C09K 11/06; H10K 85/40; H10K 50/11

(57) ABSTRACT

A highly efficient and highly reliable light-emitting device is provided. The light-emitting device includes an organic compound layer between a pair of electrodes. The organic compound layer includes a light-emitting layer, the light-emitting layer includes a first organic compound, a second organic compound, and a light-emitting substance, the first organic compound contains deuterium, and in a PL measurement of a mixed layer of the first organic compound and the second organic compound, a spectrum of an exciplex is observed at room temperature, and a spectrum of the exciplex is not observed at a temperature in a temperature range of 4 K to 80 K, inclusive.

### BACKGROUND OF THE INVENTION

### 1. Field of the Invention

One embodiment of the present invention relates to an organic compound, an organic semiconductor element, a light-emitting device, a photodiode sensor, a display module, a lighting module, a display device, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display device, a liquid crystal display device, a lighting device, a power storage device, a memory device, an image capturing device, a driving method thereof, and a manufacturing method thereof.

### 2. Description of the Related Art

Light-emitting devices (also referred to as organic EL elements) including organic compounds and utilizing electroluminescence (EL) have been put into more practical use. In the basic structure of such light-emitting devices, an organic compound layer containing a light-emitting substance is located between a pair of electrodes. Carriers are injected by application of voltage to the device, and recombination energy of the carriers is used, whereby light emission can be obtained from the light-emitting substance.

Since such light-emitting devices are of self-luminous type, display devices in which the light-emitting devices are used in pixels have higher visibility than liquid crystal display devices and do not need backlights used in liquid crystal display devices. Display devices that include such light-emitting devices are also highly advantageous in that they can be thin and lightweight. Moreover, such light-emitting devices also have a feature of extremely fast response speed.

Since light-emitting layers of such light-emitting devices can be successively formed in a planar shape, planar light emission can be achieved. This feature is difficult to realize with point light sources typified by incandescent lamps and LED lamps or linear light sources typified by fluorescent lamps; thus, the light-emitting devices also have great potential as planar light sources, which can be used for lighting devices and the like.

Display devices or lighting devices that include light-emitting devices are suitable for a variety of electronic appliances as described above, and research and development of light-emitting devices have progressed for better characteristics.

For example, Non-Patent Document 1 reports a light-emitting device that includes a platinum complex as a light-emitting dopant.

### REFERENCE

### Non-Patent Document

[Non-Patent Document 1] Jinwon Sun, et al., “Exceptionally stable blue phosphorescent organic light-emitting diodes” Nature Photonics, vol. 16, pp 212-pp 218, 2022

### SUMMARY OF THE INVENTION

An object of one embodiment of the present invention is to provide a highly reliable light-emitting device. Another object of one embodiment of the present invention is to provide a light-emitting device having high emission efficiency. Another object of one embodiment of the present invention is to provide a high-color-purity light-emitting device. Another object of one embodiment of the present invention is to provide any of a low-power-consumption display device, a low-power-consumption electronic appliance, and a low-power-consumption lighting device. Another object of one embodiment of the present invention is to provide any of a highly reliable display device, a highly reliable electronic appliance, and a highly reliable lighting device. Another object of one embodiment of the present invention is to provide any of a high-color-purity display device, a high-color-purity electronic appliance, and a high-color-purity lighting device.

It is only necessary that at least one of the above-described objects be achieved in the present invention.

One embodiment of the present invention is a light-emitting device including an organic compound layer between a pair of electrodes, the organic compound layer includes a light-emitting layer, the light-emitting layer includes a first organic compound, a second organic compound, and a light-emitting substance, and the first organic compound contains deuterium. In a photoluminescence (PL) measurement of a mixed film of the first organic compound and the second organic compound, a spectrum of an exciplex is observed at room temperature, and a spectrum of the exciplex is not observed at a temperature in a temperature range of 4 K to 80 K, inclusive.

Another embodiment of the present invention is a light-emitting device including an organic compound layer between a pair of electrodes, the organic compound layer includes a light-emitting layer, the light-emitting layer includes a first organic compound, a second organic compound, and a light-emitting substance, and the first organic compound contains deuterium. In a PL measurement of a mixed film of the first organic compound and the second organic compound, a spectrum of an exciplex is observed at room temperature, and a plurality of peaks are observed at a temperature in a temperature range of 4K to 80 K, inclusive.

Another embodiment of the present invention is a light-emitting device including an organic compound layer between a pair of electrodes, the organic compound layer includes a light-emitting layer, the light-emitting layer includes a first organic compound, a second organic compound, and a light-emitting substance, and the first organic compound contains deuterium. In a PL measurement of a mixed film of the first organic compound and the second organic compound, a spectrum of an exciplex is observed at room temperature, and a phosphorescent spectrum of the first organic compound and a phosphorescent spectrum of the second organic compound are observed at a temperature in a temperature range of 4 K to 80 K, inclusive.

Another embodiment of the present invention is a light-emitting device including an organic compound layer between a pair of electrodes, the organic compound layer includes a light-emitting layer, the light-emitting layer includes a first organic compound, a second organic compound, and a light-emitting substance, and the first organic compound contains deuterium and three or more carbazole skeletons. In a PL measurement of a mixed film of the first organic compound and the second organic compound, a plurality of peaks are observed at a temperature in a temperature range of 4 K to 80 K, inclusive.

Another embodiment of the present invention is a light-emitting device including an organic compound layer between a pair of electrodes, the organic compound layer includes a light-emitting layer, the light-emitting layer includes a first organic compound, a second organic compound, and a light-emitting substance, and the first organic compound contains a deuterated carbazole skeleton. In a PL measurement of a mixed film of the first organic compound and the second organic compound, a plurality of peaks are observed at a temperature in a temperature range of 4 K to 80 K, inclusive.

In any of the above structures, a difference between the T₁ level of the first organic compound and the T₁ level of the second organic compound is less than or equal to 0.100 eV.

In any of the above structures, the light-emitting substance is a phosphorescent substance.

In any of the above structures, the light-emitting substance is a metal complex containing platinum.

One embodiment of the present invention is an organic compound represented by General Formula (G1-1) or General Formula (G1-2).

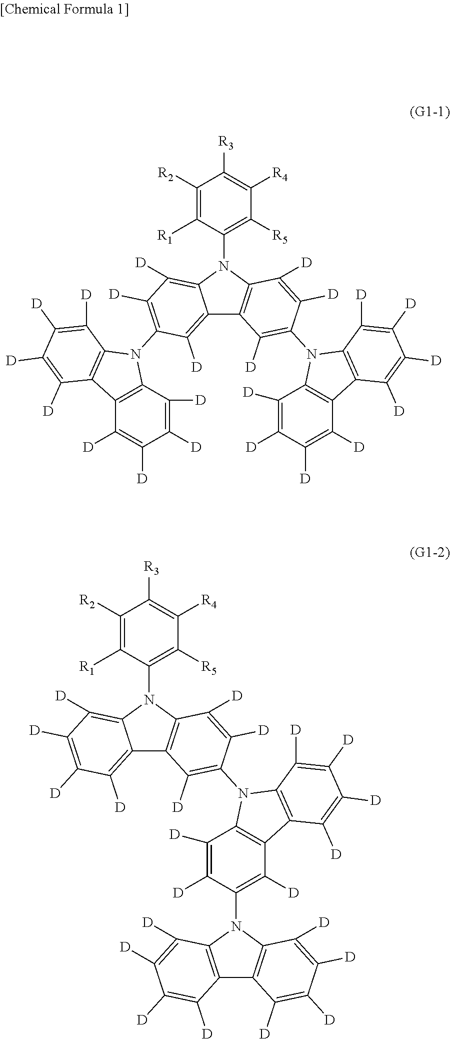

In General Formulas, R¹ to R⁵ each independently represent hydrogen, deuterium, a substituted or unsubstituted alkyl group having 1 to 10 carbon atoms, a substituted or unsubstituted cycloalkyl group having 3 to 10 carbon atoms, a substituted or unsubstituted polycyclic alkyl group having 6 to 10 carbon atoms, a substituted or unsubstituted aryl group having 6 to 30 carbon atoms, a substituted or unsubstituted heteroaryl group having 2 to 30 carbon atoms, a triarylsilyl group having 18 to 36 carbon atoms, or a trialkylsilyl group having 3 to 12 carbon atoms.

Another embodiment of the present invention is an organic compound represented by General Formula (G2) below.

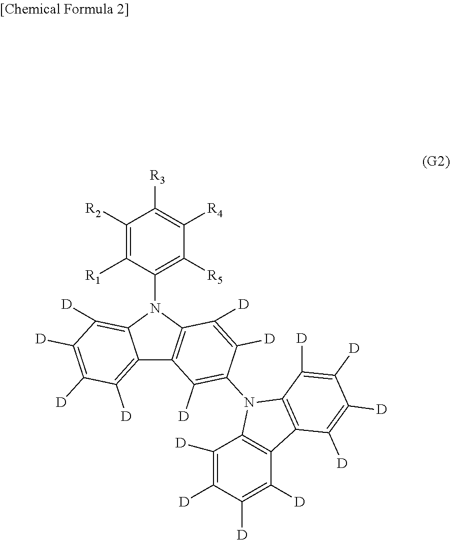

In Formula, at least one of R¹ to R⁵ represents a carbazolyl group containing deuterium, and the others of R¹ to R⁵ each independently represent each independently represent hydrogen, deuterium, a substituted or unsubstituted alkyl group having 1 to 10 carbon atoms, a substituted or unsubstituted cycloalkyl group having 3 to 10 carbon atoms, a substituted or unsubstituted polycyclic alkyl group having 6 to 10 carbon atoms, a substituted or unsubstituted aryl group having 6 to 30 carbon atoms, a substituted or unsubstituted heteroaryl group having 2 to 30 carbon atoms, a triarylsilyl group having 18 to 36 carbon atoms, or a trialkylsilyl group having 3 to 12 carbon atoms.

Another embodiment of the present invention is an organic compound represented by Structural Formula (100) or Structural Formula (101).

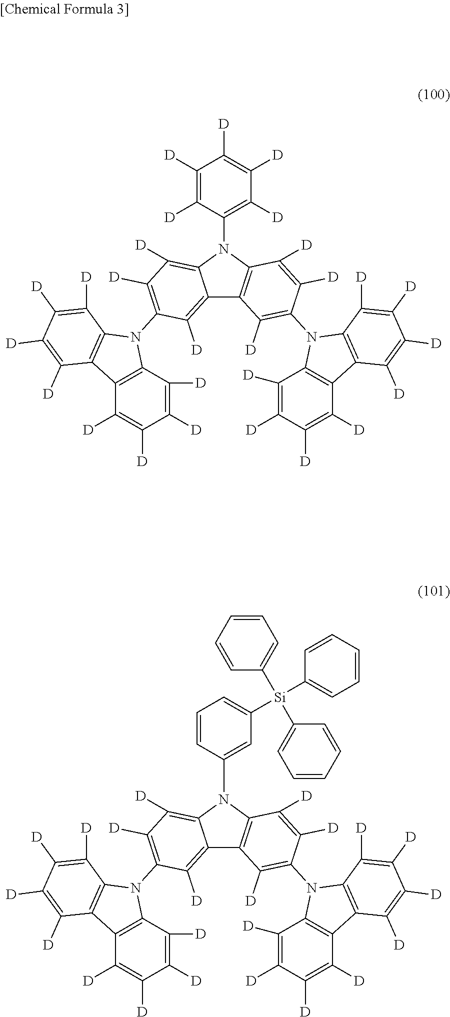 [SMILES: [2H]c1c([2H])c([2H])c(-n2c3c([2H])c([2H])c(-n4c5c([2H])c([2H])c([2H])c([2H])c5c5c([2H])c([2H])c([2H])c([2H])c54)c([2H])c3c3c([2H])c(-n4c5c([2H])c([2H])c([2H])c([2H])c5c5c([2H])c([2H])c([2H])c([2H])c54)c([2H])c([2H])c32)c([2H])c1[2H] ; [2H]c1c([2H])c([2H])c2c(c1[2H])c1c([2H])c([2H])c([2H])c([2H])c1n2-c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc([Si](c2ccccc2)(c2ccccc2)c2ccccc2)c1]

Another embodiment of the present invention is a light-emitting device including an organic compound layer between a pair of electrodes, the organic compound layer includes a light-emitting layer, and the light-emitting layer includes the organic compound described in General Formula (G1-1), General Formula (G1-2), General Formula (G2), Structural Formula (100), or Structural Formula (101).

Another embodiment of the present invention is a display device including any of the above light-emitting devices.

Another embodiment of the present invention is an electronic appliance that includes the above light-emitting device and at least one of a sensor, an operation button, a speaker, and a microphone.

Another embodiment of the present invention is a lighting device that includes the above light-emitting device and a housing.

According to one embodiment of the present invention, a highly reliable light-emitting device can be provided. According to another embodiment of the present invention, a light-emitting device having high emission efficiency can be provided. According to another embodiment of the present invention, a light-emitting device having high color purity can be provided. According to another embodiment of the present invention, any of a low-power-consumption display device, a low-power-consumption electronic appliance, and a low-power-consumption lighting device can be provided. According to another embodiment of the present invention, any of a highly reliable display device, a highly reliable electronic appliance, and a highly reliable lighting device can be provided. According to another embodiment of the present invention, any of a high-color-purity display device, a high-color-purity electronic appliance, and a high-color-purity lighting device can be provided.

Note that the description of these effects does not preclude the existence of other effects. One embodiment of the present invention does not necessarily have all these effects. Other effects will be apparent from and can be derived from the description of the specification, the drawings, the claims, and the like.

### BRIEF DESCRIPTION OF THE DRAWINGS

In the accompanying drawings:

FIGS. 1A and 1B schematically illustrate a light-emitting device;

FIG. 2A is a conceptual diagram of energy transfer of a light-emitting device of one embodiment of the present invention and FIG. 2B is a conceptual diagram of energy transfer of a conventional light-emitting device;

FIGS. 3A to 3E each illustrate a structure of a light-emitting device;

FIGS. 4A and 4B are a top view and a cross-sectional view, respectively, of a light-emitting apparatus;

FIGS. 5A to 5D each illustrate a light-emitting device;

FIGS. 6A to 6E are cross-sectional views illustrating an example of a method for manufacturing a light-emitting apparatus;

FIGS. 7A to 7E are cross-sectional views illustrating an example of a method for manufacturing a light-emitting apparatus;

FIGS. 8A to 8C are cross-sectional views illustrating an example of a method for manufacturing a light-emitting apparatus;

FIGS. 9A to 9C are cross-sectional views illustrating an example of a method for manufacturing a light-emitting apparatus;

FIGS. 10A to 10C are cross-sectional views illustrating an example of a method for manufacturing a light-emitting apparatus;

FIGS. 11A to 11C are cross-sectional views illustrating an example of a method for manufacturing a light-emitting apparatus;

FIGS. 12A to 12C are cross-sectional views illustrating an example of a method for manufacturing a light-emitting apparatus;

FIGS. 13A to 13G are top views each illustrating a structure example of a pixel;

FIGS. 14A to 141 are top views each illustrating a structure example of a pixel;

FIGS. 15A and 15B are perspective views illustrating a structure example of a display module;

FIGS. 16A and 16B are cross-sectional views each illustrating a structure example of a light-emitting apparatus;

FIG. 17 is a perspective view illustrating a structure example of a light-emitting apparatus;

FIG. 18A is a cross-sectional view illustrating a structure example of a light-emitting apparatus and FIGS. 18B and 18C are cross-sectional views illustrating structure examples of a transistor;

FIG. 19 is a cross-sectional view illustrating a structure example of a light-emitting apparatus;

FIGS. 20A to 20D are cross-sectional views each illustrating a structure example of a light-emitting apparatus;

FIGS. 21A to 21D each illustrate an example of an electronic appliance;

FIGS. 22A to 22F each illustrate an example of an electronic appliance;

FIGS. 23A to 23G each illustrate an example of an electronic appliance;

FIG. 24 shows absorption and emission spectra of an organic compound formed in an example;

FIG. 25 illustrates a structure of a light-emitting device in an example;

FIG. 26 is a graph showing luminance-current density characteristics of light-emitting devices in an example;

FIG. 27 is a graph showing luminance-voltage characteristics of light-emitting devices in an example;

FIG. 28 is a graph showing current efficiency-luminance characteristics of light-emitting devices in an example;

FIG. 29 is a graph showing the current density-voltage characteristics of light-emitting devices in an example;

FIG. 30 is a graph showing power efficiency-luminance characteristics of light-emitting devices in an example;

FIG. 31 is a graph showing external quantum efficiency-luminance characteristics of light-emitting devices in an example;

FIG. 32 is a graph showing an electroluminescence spectrum of light-emitting devices in an example;

FIG. 33 shows driving time-dependent changes in luminance of light-emitting devices in an example;

FIG. 34 shows LT80 of light-emitting devices in an example;

FIGS. 35A and 35B show PL spectra at room temperature and 10 K of materials used in an example;

FIGS. 36A and 36B show PL spectra at room temperature and 10 K of materials used in an example;

FIGS. 37A and 37B show PL spectra at room temperature and 10 K of materials used in an example;

FIGS. 38A and 38B show PL spectra at room temperature and 10 K of materials used in an example;

FIG. 39 show PL spectra at 10 K of materials used in an example;

FIG. 40 shows absorption and emission spectra of an organic compound formed in an example;

FIG. 41 shows absorption and emission spectra of a mixed film including an organic compound formed in an example; and

FIGS. 42A and 42B show PL spectra at 10 K of a mixed film including an organic compound formed in an example.

### DETAILED DESCRIPTION OF THE INVENTION

Embodiments of the present invention will be described in detail below with reference to the drawings. Note that the embodiments of the present invention are not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

In this specification and the like, a device formed using a metal mask or a fine metal mask (FMM) is sometimes referred to as a device having a metal mask (MM) structure. In this specification and the like, a device formed without using a metal mask or an FMM is sometimes referred to as a device having a metal maskless (MML) structure.

### Embodiment 1

It is a long time since displays (organic EL displays) that include organic EL elements (hereinafter also referred to as light-emitting devices) as display elements were put into practical use. These displays usually include pixels emitting light with at least three colors of red, green, and blue to achieve full-color display.

In a display fabricated by a side-by-side method or a side-by-side patterning method, in which light-emitting devices for the respective emission colors are provided in the pixels, the light-emitting devices include light-emitting substances corresponding to the respective emission colors of the pixels.

It is known that in current-excitation type organic EL devices, the theoretical limit of the internal quantum efficiency of a light-emitting device using a fluorescent material, which can utilize only a singlet excited state for light emission, is 25% since the generation probability ratio of a singlet excited state to that of a triplet excited state is 1:3. By contrast, a phosphorescent material can convert a singlet excited state into a triplet excited state through intersystem crossing and thus enables a light-emitting device with an internal quantum efficiency of 100% theoretically, which allows the light-emitting device to have higher emission efficiency than the case of using the fluorescent material. This is why phosphorescent materials are used as light-emitting substances in many red- and green-light-emitting devices in currently commercialized organic EL displays.

However, even in displays using phosphorescent materials for red and green light-emitting devices, in many cases, a fluorescent material that is inferior in efficiency to the phosphorescent materials is used for a blue-light-emitting device. A reason for this lies in not efficiency but reliability. Generally, light-emitting devices using a phosphorescent material as a blue-light-emitting substance have short lifetimes and have difficulty in achieving high reliability.

The reliability is decreased, for example, because the phosphorescent material has a long emission lifetime (also referred to as phosphorescence lifetime or exciton lifetime). Transition from a triplet excited state to a singlet ground state is spin-forbidden, whereas transition from a singlet excited state to a singlet ground state is spin-allowed; thus, the phosphorescence lifetime (˜μs) is much longer than the fluorescence lifetime (˜ns). A long phosphorescence lifetime means a long lifetime of a triplet exciton. Therefore, in a phosphorescent light-emitting device, a light-emitting substance keeps being in a high-energy excited state for a long time, which a cause of promoting deterioration of the light-emitting substance or nearby substances.

In the case where an organic compound (also referred to as a host material) to disperse a light-emitting substance is used in a light-emitting layer, the host material is preferably a substance having a triplet excited level and a single excited level that are higher than the triplet exited level and the single excited level of the light-emitting substance (also referred to as a guest material). In addition, blue light emission energy is higher than red or green light emission energy, and thus, a host material to disperse a blue-light-emitting substance preferably have a triplet excited state and a singlet excited state that are higher than those of a red- or green-light-emitting substance. Accordingly, the choice of the material is limited and it is difficult to obtain a material having high performance.

As described above, the energy of the triplet excited state of a light-emitting device including a phosphorescent material for blue light is higher than the energies of the triplet excited states of phosphorescent materials for red and green; thus, the light-emitting device including the phosphorescent material for blue light is significantly influenced by the long exciton lifetime of the light-emitting substance compared to a light-emitting device including a light-emitting substance for red or green; as a result, the light-emitting device including the phosphorescent material for blue light is yet to have the reliability high enough for practical use.

One embodiment of the present invention provides a light-emitting device using a highly reliable phosphorescent light-emitting substance or an organic compound that can be used in the light-emitting device. Next, a structure of the light-emitting device of one embodiment of the present invention will be described.

### <Structure Example of Light-Emitting Device>

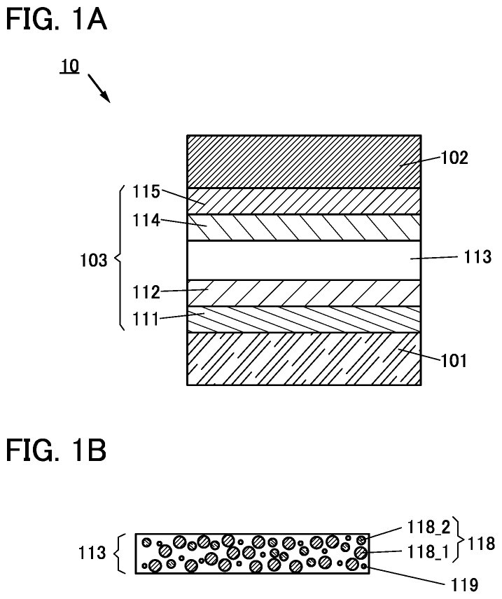

FIG. 1A is a schematic cross-sectional view of a light-emitting device 10 of one embodiment of the present invention. The light-emitting device 10 includes a pair of electrodes (a first electrode 101 and a second electrode 102) and an organic compound layer 103 between the pair of electrodes. The organic compound layer 103 includes at least a light-emitting layer 113.

The organic compound layer 103 illustrated in FIG. 1A includes functional layers such as a hole-injection layer 111, a hole-transport layer 112, an electron-transport layer 114, and an electron-injection layer 115, in addition to the light-emitting layer 113.

Although description in this embodiment is made assuming that the first electrode 101 and the second electrode 102 of the pair of electrodes serve as an anode and a cathode, respectively, the structure of the light-emitting device 10 is not limited thereto. That is, the first electrode 101 may be a cathode, the second electrode 102 may be an anode, and the stacking order of the layers between the electrodes may be reversed. In other words, the hole-injection layer 111, the hole-transport layer 112, the light-emitting layer 113, the electron-transport layer 114, and the electron-injection layer 115 may be stacked in this order from the anode side.

The structure of the organic compound layer 103 is not limited to the structure illustrated in FIG. 1A, and a structure including at least one layer selected from the hole-injection layer 111, the hole-transport layer 112, the electron-transport layer 114, and the electron-injection layer 115 may be employed. Alternatively, the organic compound layer 103 may include a functional layer which has a function of lowering a hole- or electron-injection barrier, improving a hole- or electron-transport property, inhibiting a hole- or electron-transport property, or reducing quenching by an electrode, for example. Note that the functional layer may be either a single layer or stacked layers.

FIG. 1B is a schematic cross-sectional view illustrating an example of the light-emitting layer 113 in FIG. 1A. The light-emitting layer 113 illustrated in FIG. 1B contains host materials 118 (an organic compound 118_1 and an organic compound 118_2) and a guest material 119 (a light-emitting substance).

The guest material 119 may be a light-emitting organometallic complex, and the light-emitting organometallic complex is preferably a substance capable of emitting phosphorescent light (hereinafter also referred to as a phosphorescent compound).

In the light-emitting layer 113, the host materials 118 are present in the largest proportion by weight, and the guest material 119 (phosphorescent compound) is dispersed in the host materials 118. As described above, the lowest triplet excited level (T₁ level) of the host materials 118 (the organic compound 118_1 and the organic compound 118_2) in the light-emitting layer 113 are preferably higher than the T₁ level of the guest material 119 in the light-emitting layer 113.

The host materials 118 (the organic compound 118_1 and the organic compound 118_2) in the light-emitting layer 113 preferably form an exciplex. An exciplex is an excited state formed from two or more kinds of substances. In photoexcitation, the exciplex is formed by interaction between one substance in an excited state and another substance in a ground state. The two kinds of substances that have formed the exciplex return to a ground state by emitting light and then serve as the original two kinds of substances. Because the excited energy level of the exciplex is lower than the singlet excited energy levels of the host materials 118 (the organic compound 118_1 and the organic compound 118_2) that form the exciplex, the excited state of the host materials 118 can be formed with lower excited energy. Accordingly, the driving voltage of a light-emitting device can be reduced.

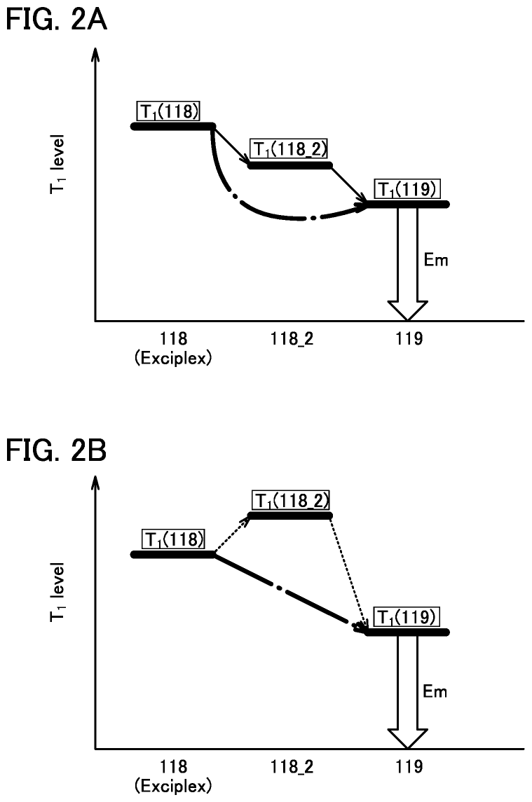

FIG. 2A is a conceptual diagram of energy transfer in light emission process of the guest material 119 in the light-emitting device of one embodiment of the present invention. The T₁ level (T₁(118_2)) energy of the organic compound 118_2 is lower than the T₁ level (T₁(118)) energy of an exciplex formed by the host materials 118 (the organic compound 118_1 and the organic compound 118_2) and is higher than the T₁ level (T₁(119)) energy of the guest material 119 serving as a light-emitting substance.

In a light-emitting device in which an exciplex formed by the host materials 118 is utilized, excitation energy is basically transferred from the T₁ level of the exciplex formed by excitation of the host materials 118 to the T₁ level of the guest material 119 (as indicated by a dashed-dotted arrow), so that the guest material 119 is excited. The excited guest material 119 returns from the triplet excited state to the ground state, so that light emission (Em) is generated.

In the light-emitting device of one embodiment of the present invention, the T₁ level energy of the organic compound 1182 is lower than the T₁ level energy of the exciplex and is higher than the T₁ level energy of the guest material 119, whereby excitation energy transfer from the T₁ level of the exciplex to the T₁ level of the guest material 119 through the T₁ level of the organic compound 118_2 (as indicated by the solid line arrow) as well as the above-described energy transfer from the T₁ level of the exciplex to the T₁ level of the guest material 119 (as indicated by the dashed-dotted arrow) is enabled. In this manner, energy of the exciplex can be transferred efficiently.

FIG. 2B is a conceptual diagram of energy transfer in a conventional light-emitting device. The T₁ level (T₁(118_2)) energy of the organic compound 118_2 is higher than the T₁ level (T₁(118)) energy of the exciplex formed by the host materials 118 (the organic compound 118_1 and the organic compound 118_2) and is higher than the T₁ level energy of (T₁(119)) of the guest material 119 serving as the light-emitting substance.

In other words, in the conventional light-emitting device, excitation energy is not transferred from the T₁ level of the exciplex to the T₁ level of the organic compound 118_2. Since the organic compound 118_2 is not excited, excitation energy transfer from the T₁ level of the organic compound 118_2 to the T₁ level of the guest material 119 (indicated by the broken arrows) is not caused. That is, in the conventional light-emitting device, energy is directly transferred from the T₁ level of the exciplex to the T₁ level of the guest material 119 (as indicated by the dashed-dotted arrow), so that the guest material 119 is excited.

In contrast, the light-emitting device of one embodiment of the present invention has a plurality of energy transfer paths from the T₁ level of the exciplex to the guest material 119, in which energy can be efficiently transferred to the guest material 119 before the exciplex deactivates. Accordingly, the light-emitting device of one embodiment of the present invention can exhibit high emission efficiency and include a highly reliable phosphorescent material.

As for excited levels, energy at an absorption edge of an absorption spectrum or an emission edge on the short wavelength side of a fluorescent spectrum (fluorescent component) of a photoluminescence (PL) spectrum at room temperature (e.g., approximately 290 K to 300 K (17° C. to 27° C.), preferably around 298 K (25° C.)) can be regarded as energy of a singlet excited level, and energy at an emission edge on the short wavelength side of a phosphorescent spectrum (phosphorescent component) of a PL spectrum at a low temperature (a temperature in the temperature range of 4 K to 80 K, inclusive) can be regarded as energy of a triplet excited level.

Measurement of each of the spectra can be performed on samples in a thin-film state or in a solution state. Note that comparison of the spectra themselves is performed on samples that are all in the same state, like thin-film samples or samples in a solution. In other words, when a PL spectrum at room temperature and a PL spectrum at a low temperature (a temperature in the temperature range of 4 K to 80 K, inclusive) are compared, the comparison is preferably performed on the samples that are all in the same state such as a thin-film state or solution state.

In the case where the measurement is performed on a sample in a solution state, hexane, benzene, toluene, diethyl ether, ethyl acetate, chloroform, chlorobenzene, dichloromethane, 2-methyltetrahydrofuran (2-MeTHF), or the like can be used as a solvent; toluene, dichloromethane, or 2-MeTHF is preferred. Furthermore, in observation of a phosphorescent component at a low temperature, a mixed solvent where iodobenzene:dichloromethane:toluene=20%:40%:40%, or the like may be used, in which case a favorable glass state can be formed.

Note that in observation of the phosphorescent component in a solution state, the measurement is preferably performed at 77K to 80K since the temperature of liquid nitrogen is 77K. In observation of the phosphorescent component in a thin-film state, the measurement is preferably performed at a temperature in a range from 4 K to 10 K because the temperature of the liquid helium is 4 K.

The lowest triplet excited state (T₁ level) of an organic compound can be energy of the intersection of the horizontal axis (wavelength) or the base line and a tangent to the phosphorescent spectrum at a point where the slope of the phosphorescent spectrum at a peak on the shorter wavelength side has a maximum value (see Daisaku TANAKA et al., “Ultra High Efficiency Green Organic Light-Emitting Devices”, Japanese Journal of Applied Physics, Vol. 46, No. 1, 2007, pp. L10-L12, for example). As another method, when the ν=0→ν=0 transition (0→0 band) between vibrational levels of the ground state and the excited state is clearly observed from a phosphorescent spectrum, the T₁ level can also be calculated using the 0→0 band (see Nicholas J. Turro, V. Ramamurthy, J. C. Scaiano, “MODERN MOLECULAR PHOTOCHEMISTRY OF ORGANIC MOLECULES”, UNIVERSITY SCIENCE BOOKS, published on Feb. 10, 2010, pp. 204-208). In this specification, the former method of drawing a tangent is employed to calculate the levels. In the case where the levels are compared with each other, those calculated by the same method are used.

Furthermore, it is highly likely that a light-emitting substance is in an excited state with high energy for a long time in a phosphorescent light-emitting device and thus promotes degradation of the host materials 118 in the surroundings of the light-emitting substance. In particular, the organic compound 118_2 used in one embodiment of the present invention receives energy from the exciplex of the host materials 118 and thus is excited alone. Accordingly, the organic compound 118_2 is preferably formed using a compound having a stable structure in an excited state.

Specifically, an organic compound containing deuterium is preferably used as the organic compound 118_2. Deuterium atoms are bonded to carbon atoms with a high spin density in the triplet excited state, so that the organic compound can have a stable structure in an excited state and improved reliability. The vibration of the organic compound can be suppressed in the excited state and nonradiative deactivation from the excited state can be suppressed. High emission efficiency can be achieved.

### <Example of Organic Compound>

Next, examples of an organic compound that can be used for the light-emitting device of one embodiment of the present invention will be described. The organic compound described below is preferably used as the organic compound 118_2, in particular.

### Compound Example 1

One embodiment of the present invention is an organic compound represented by General Formula (G1-1) or General Formula (G1-2).

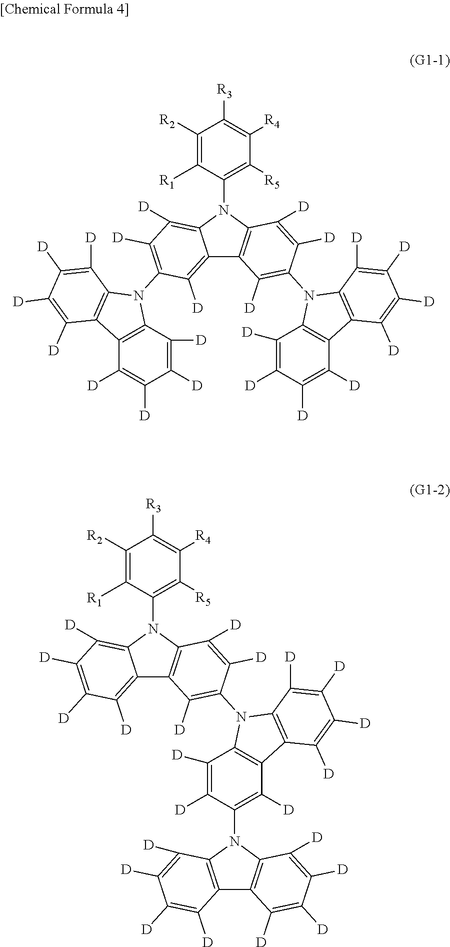

In General Formula (G1-1) or General Formula (G1-2), R¹ to R⁵ each independently represent hydrogen, deuterium, a substituted or unsubstituted alkyl group having 1 to 10 carbon atoms, a substituted or unsubstituted cycloalkyl group having 3 to 10 carbon atoms, a substituted or unsubstituted polycyclic alkyl group having 6 to 10 carbon atoms, a substituted or unsubstituted aryl group having 6 to 30 carbon atoms, a substituted or unsubstituted heteroaryl group having 2 to 30 carbon atoms, a triarylsilyl group having 18 to 36 carbon atoms, or a trialkylsilyl group having 3 to 12 carbon atoms.

### Compound Example 2

One embodiment of the present invention is an organic compound represented by General Formula (G2).

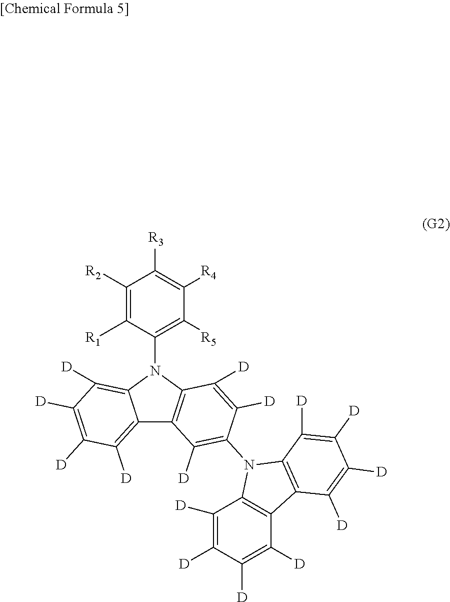

In General Formula (G2), at least one of R¹ to R⁵ represents a deuterated carbazolyl group, the others of R¹ to R⁵ each independently represent hydrogen, deuterium, a substituted or unsubstituted alkyl group having 1 to 10 carbon atoms, a substituted or unsubstituted cycloalkyl group having 3 to 10 carbon atoms, a substituted or unsubstituted polycyclic alkyl group having 6 to 10 carbon atoms, a substituted or unsubstituted aryl group having 6 to 30 carbon atoms, a substituted or unsubstituted heteroaryl group having 2 to 30 carbon atoms, a triarylsilyl group having 18 to 36 carbon atoms, or a trialkylsilyl group having 3 to 12 carbon atoms.

Examples of the alkyl group substituted for R¹ to R⁵ in General Formula (G1-1), General Formula (G1-2), and General Formula (G2) above include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a 3-methylpentyl group, a 2-methylpentyl group, a 2-ethylbutyl group, a 1,2-dimethylbutyl group, and a 2,3-dimethylbutyl group.

Examples of the cycloalkyl group substituted for R¹ to R⁵ in General Formula (G1-1), General Formula (G1-2), and General Formula (G2) above include a cyclopropyl group, a cyclobutyl group, a cyclopentyl group, a cyclohexyl group, a cycloheptyl group, a cyclooctyl group, a cyclononanyl group, a cyclodecyl group, and a cyclododecyl group. Examples of the cycloalkyl group having 4 to 10 carbon atoms and having a bridged structure include a bicyclobutyl group, a noradamantyl group, an adamantyl group, a norbornanyl group, and a tetrahydrodicyclopentadienyl group.

Examples of the polycyclic alkyl group substituted for R¹ to R⁵ in General Formula (G1-1), General Formula (G1-2), and General Formula (G2) above can include a cyclopropyl group, a cyclobutyl group, a cyclopentyl group, a cyclohexyl group, a 1-methylcyclohexyl group, a cycloheptyl group, and an adamantyl group.

Examples of the aryl group substituted for R¹ to R⁵ in General Formula (G1-1), General Formula (G1-2), and General Formula (G2) include a phenyl group, a tolyl group, a xylyl group, a biphenyl group, an indenyl group, a naphthyl group, a fluorenyl group, a spirofluorenyl group, a phenanthrenyl group, and a triphenylenyl group.

Examples of the heteroaryl group substituted for R¹ to R⁵ in General Formula (G1-1), General Formula (G1-2), and General Formula (G2) include a group including a triazine ring, a group including a pyrimidine ring, a group including a pyridine ring, a group including a phenanthroline ring, a group including a carbazole ring, a group including a dibenzofuran ring, a group including a dibenzothiophene ring, a group including a benzonaphthofuran ring, a group including a benzonaphthothiophene ring, a group including an indolocarbazole ring, a group including a benzofurocarbazole ring, a group including a benzothienocarbazole ring, a group including an indenocarbazole ring, and a group including a dibenzocarbazole ring.

Examples of the trialkylsilyl group substituted for R¹ to R⁵ in General Formula (G1-1), General Formula (G1-2), and General Formula (G2) include a trimethylsilyl group, a triethylsilyl group, and a tert-butyl dimethylsilyl group.

Examples of the triarylsilyl group substituted for R¹ to R⁵ in General Formula (G1-1), General Formula (G1-2), and General Formula (G2) include a triphenylsilyl group and a trinaphthylsilyl group.

Specifically, in General Formula (G1-1), General Formula (G1-2), and General Formula (G2), R¹ to R⁵ can each be represented by any one of General Formula (Ar-1) to General Formula (Ar-56) below. In General Formula (Ar-1) to General Formula (Ar-56), hydrogen may be replaced with deuterium as appropriate.

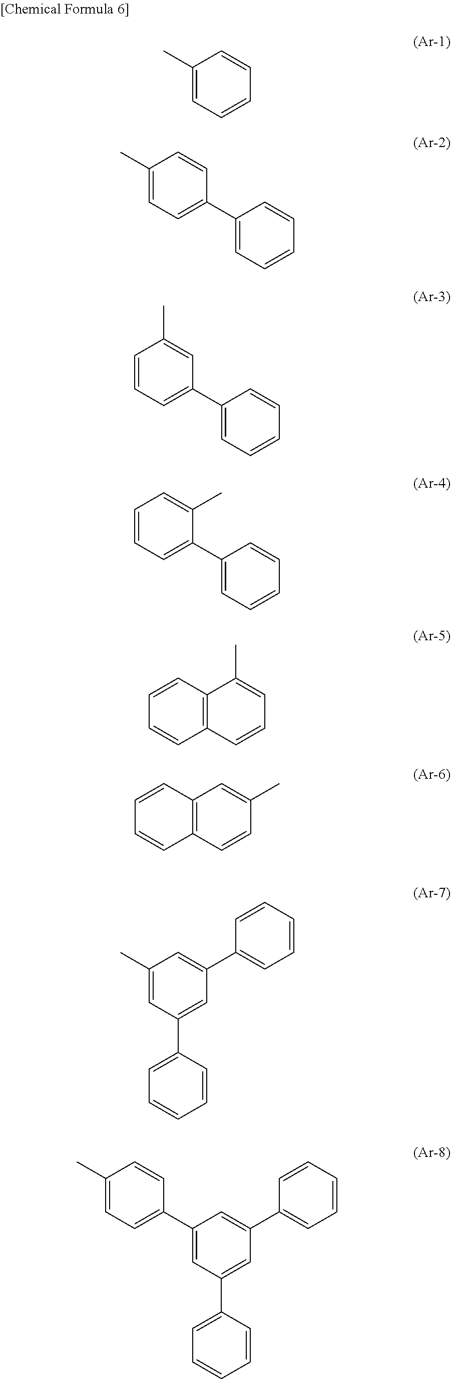

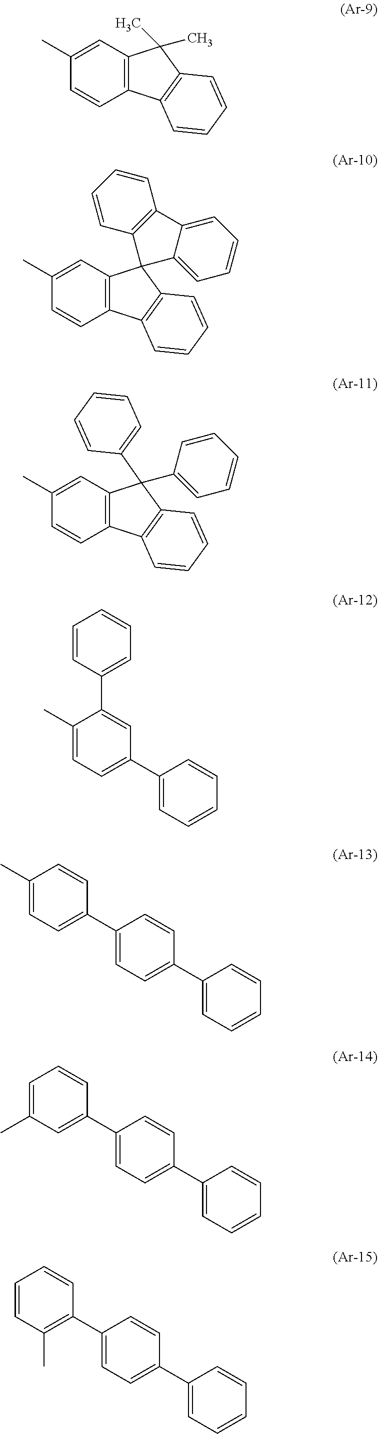

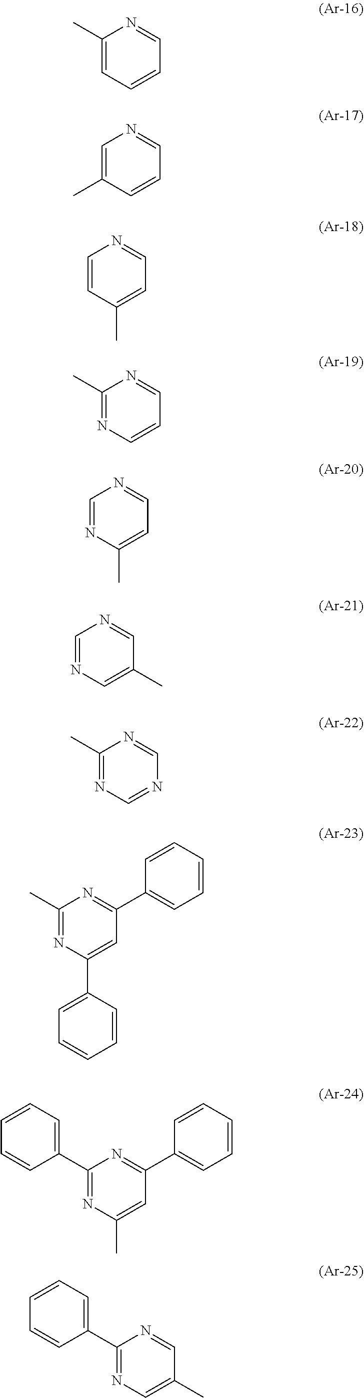

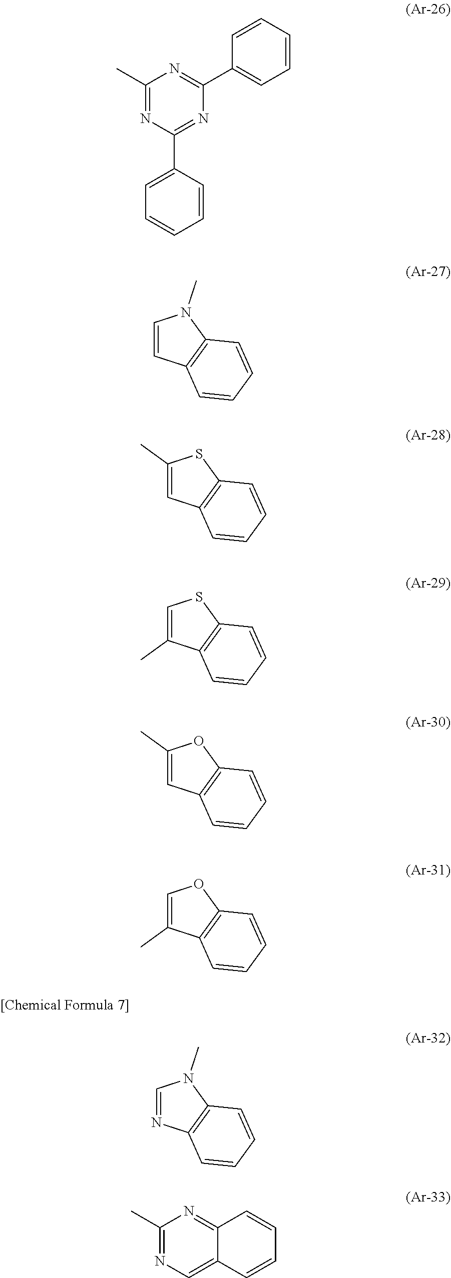

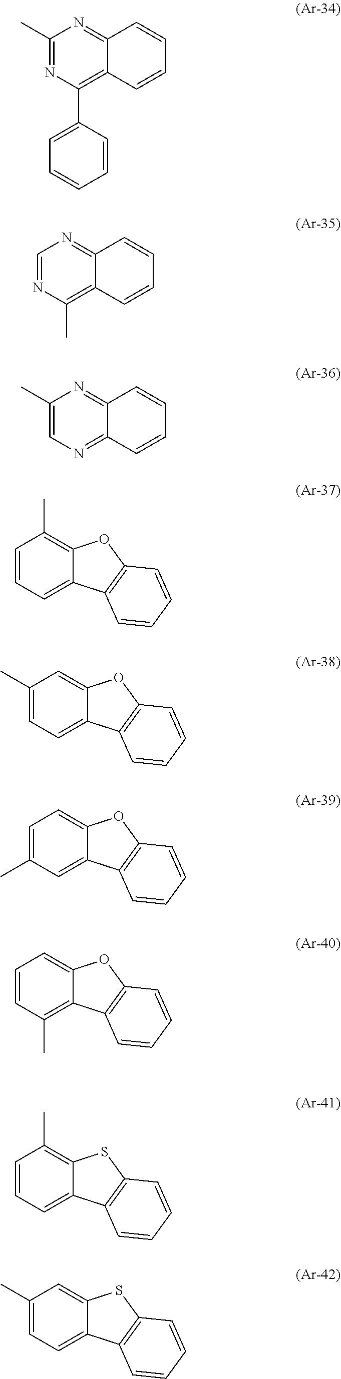

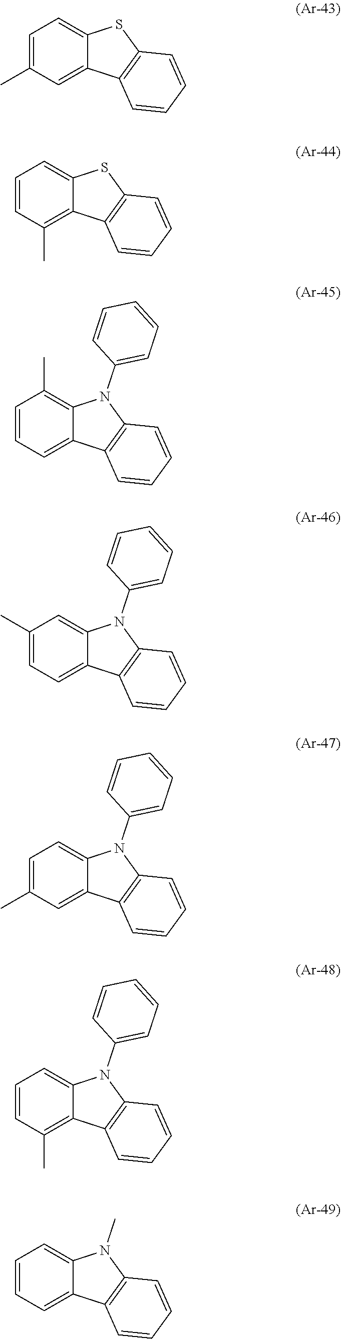

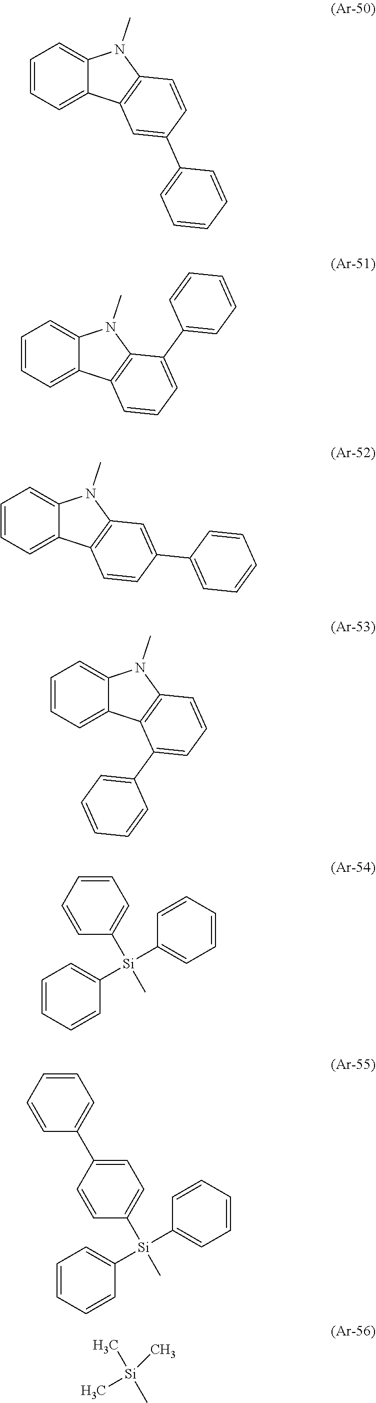

Note that General Formula (Ar-1) to General Formula (Ar-56) are non-limiting examples of R¹ to R⁵.

### SPECIFIC EXAMPLES

Shown below are specific examples of the organic compound of one embodiment of the present invention having any of the structures represented by General Formula (G1-1), General Formula (G1-2), and General Formula (G2) above.

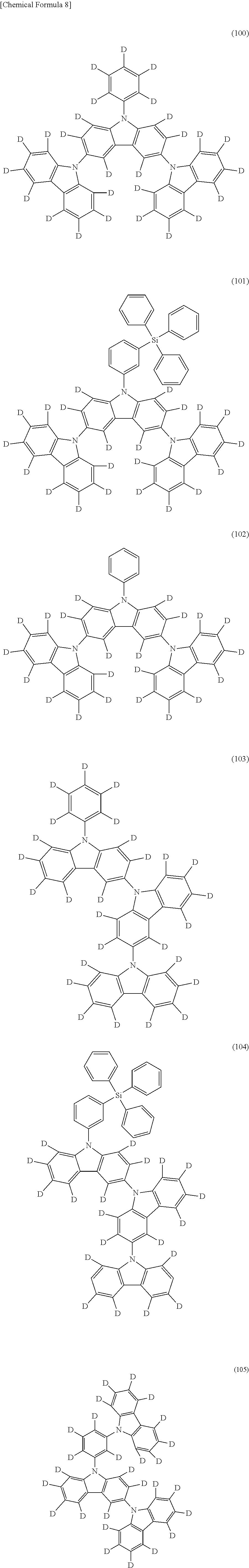

The organic compounds represented by Structural Formulas (100) to (105) above are examples of the organic compounds represented by General Formula (G1-1), General Formula (G1-2), or General Formula (G2). The organic compound of one embodiment of the present invention is not limited thereto.

### <Synthesis Method of Organic Compound>

A method of synthesizing the organometallic complex represented by General Formula (G1-1), General Formula (G1-2), or General Formula (G2) is described below. A variety of reactions can be applied to the synthesis method of the compound.

### <<Synthesis Method of Organic Compound Represented by General Formula (G1-1)>>

The organic compound of the present invention represented by General Formula (G1-1) can be synthesized as in Synthesis Schemes (s1-1) and (s1-3) below.

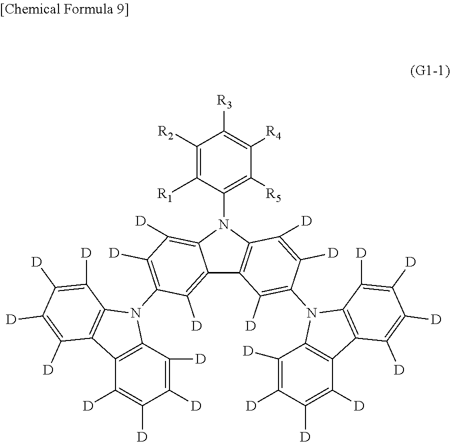

First, Synthesis Scheme (s1-1) is described. That is, Compound 1 is coupled with Compound 2 to give Compound 3. Synthesis Scheme (s1-1) is shown below.

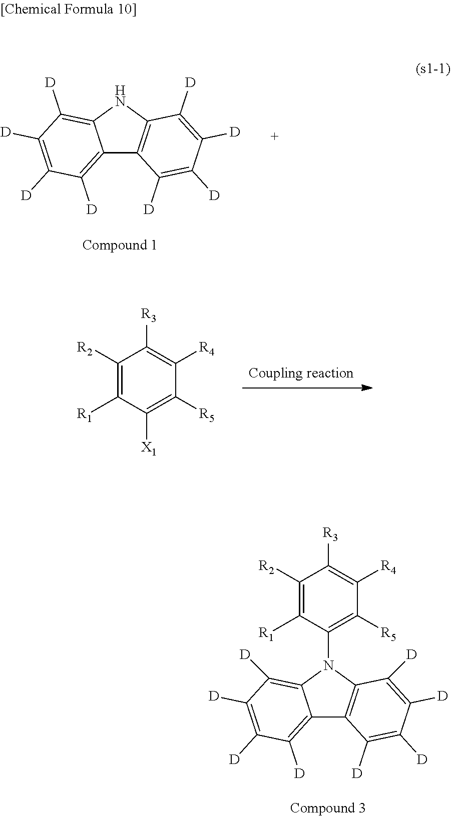

Next, Synthesis Scheme (s1-2) is described. That is, Compound 3 is halogenated to give a target substance, Compound 4. Synthesis Scheme (s1-2) is shown below.

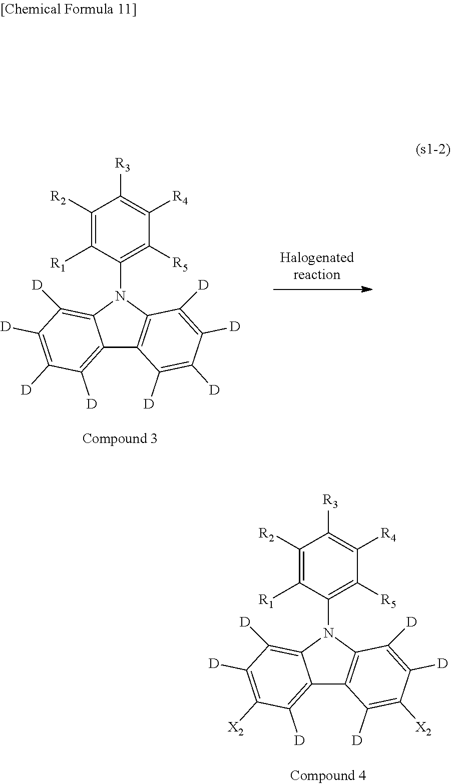

Next, Synthesis Scheme (s1-3) is described. That is, Compound 4 is coupled with Compound 1 to give the target substance represented by General Formula (G1-1). Synthesis Scheme (s1-3) is shown below.

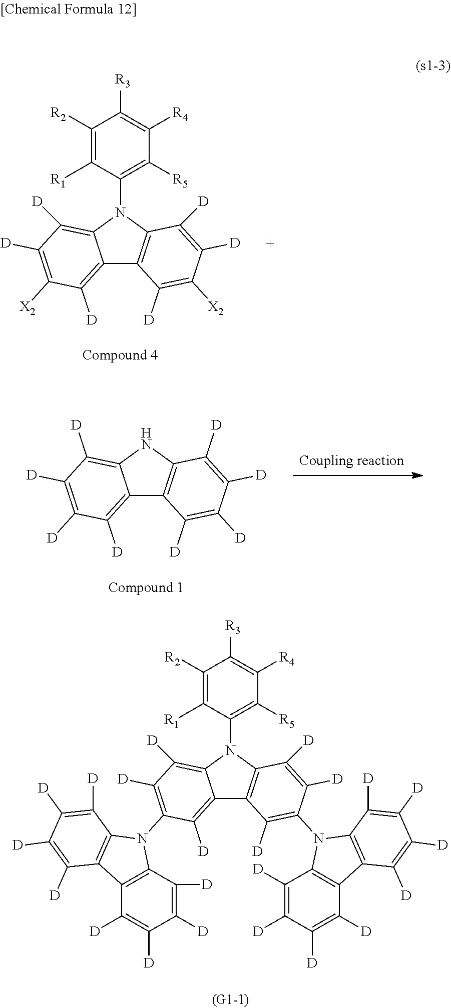

<<Synthesis Method of Organic Compound Represented by General Formula (G1-2)>>

The organic compound of the present invention represented by General Formula (G1-2) can be synthesized as in Synthesis Schemes (s2-1) and (s2-2) below.

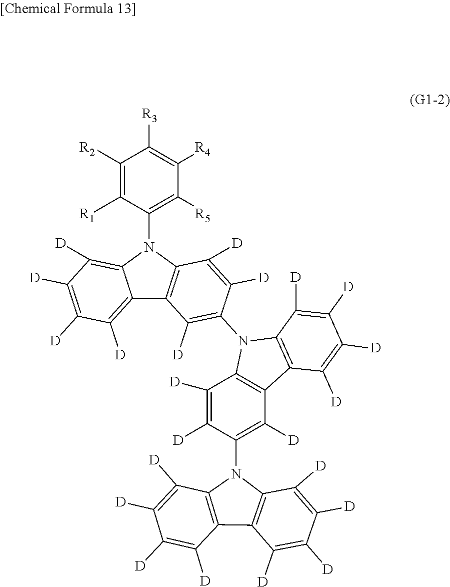

Synthesis Scheme (s2-1) is described. That is, Compound 5 is deuterated to give Compound 6. Synthesis Scheme (s2-1) is shown below.

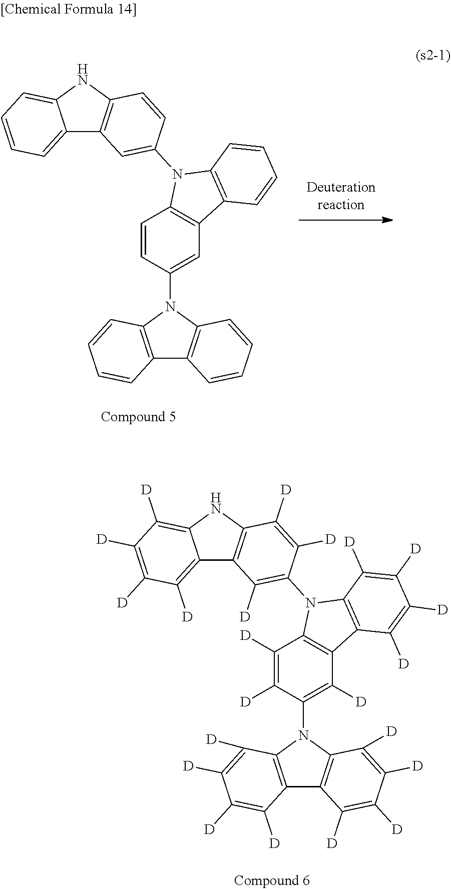 [SMILES: c1ccc2c(c1)[nH]c1ccc(-n3c4ccccc4c4cc(-n5c6ccccc6c6ccccc65)ccc43)cc12 ; [2H]c1c([2H])c([2H])c2c([nH]c3c([2H])c([2H])c(-n4c5c([2H])c([2H])c([2H])c([2H])c5c5c([2H])c(-n6c7c([2H])c([2H])c([2H])c([2H])c7c7c([2H])c([2H])c([2H])c([2H])c76)c([2H])c([2H])c54)c([2H])c32)c1[2H]]

Next, Synthesis Scheme (s2-2) is described. That is, Compound 6 is coupled with Compound 2 to give the target substance represented by General Formula (G1-2). Synthesis Scheme (s2-2) is shown below.

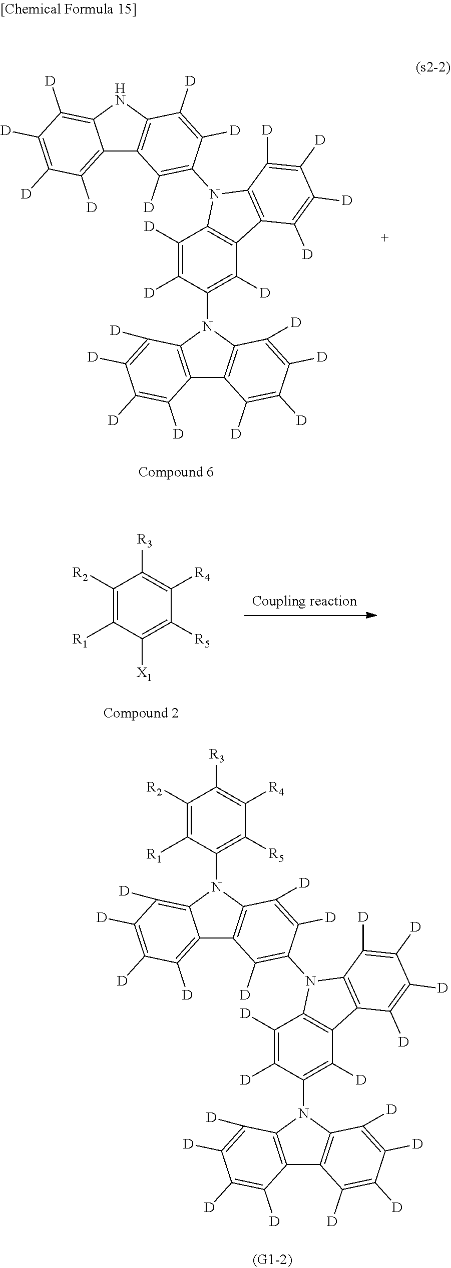

### <<Synthesis Method of Organic Compound Represented by General Formula (G2)>>

The organic compound represented by General Formula (G2) can be synthesized under Synthesis Scheme (s3-1) below.

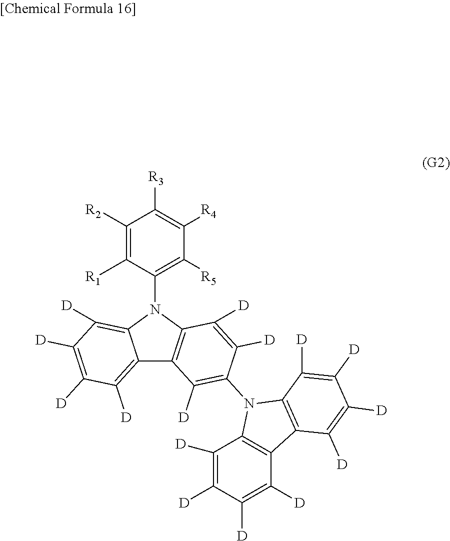

Next, Synthesis Scheme (A3) is described. That is, Compound 7 is coupled with Compound 2 to give the target substance represented by General Formula (G2). Synthesis Scheme (s3-1) is shown below.

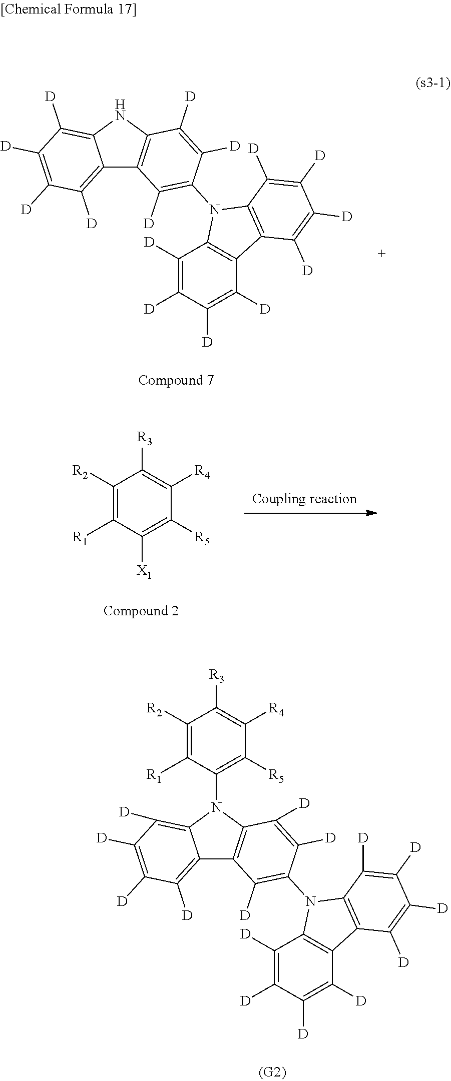

In Synthesis Schemes (s1-1) to (s1-3), Synthesis Scheme (s2-1), Synthesis Scheme (s2-2), and Synthesis Scheme (s3-1), X₁ to X₃ each independently represent fluorine, chlorine, bromine, or iodine, and preferably bromine or iodine in consideration of reactivity or preferably chlorine or bromine in consideration of cost.

Non-limiting examples of the halogenating agent that can be used in the halogenation reaction in the Synthesis Scheme (s1-2) include chlorine, bromine, iodine, N-chlorosuccinimide, N-bromosuccinimide, and N-iodosuccinimide.

Furthermore, non-limiting examples of the solvent that can be used in the halogenation reaction in Synthesis Scheme (s1-2) include chloroform, dichloromethane, ethyl acetate, and N,N-dimethylformamide.

Non-limiting examples of the solvent that can be used in the deuteration reaction in Synthesis Scheme (s2-1) include benzene-d6, toluene-d8, xylene-d10, DMSO-d6, acetonitrile-d3, and heavy water.

Non-limiting examples of the catalyst that can be used in the deuteration reaction in Synthesis Scheme (s2-1) include molybdenum(V) chloride, tungsten(VI) chloride, niobium(V) chloride, tantalum(V) chloride, aluminum(III) chloride, titanium(IV) chloride, and tin(IV) chloride.

In the case where the Buchwald-Hartwig reaction using a palladium catalyst is employed in Synthesis Schemes (s1-1) to (s1-3), Synthesis Scheme (s2-1) Synthesis Scheme (s2-2), and Synthesis Scheme (s3-1), a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride (dimer) and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, or (S)-(6,6′-dimethoxybiphenyl-2,2′-diyl)bis(diisopropylphosphine) (abbreviation: cBRIDP), can be used. In the reaction, an organic base such as sodium tert-butoxide, an inorganic base such as potassium carbonate, cesium carbonate, or sodium carbonate, or the like can be used. In the reaction, toluene, xylene, benzene, tetrahydrofuran, dioxane, or the like can be used as a solvent. Reagents that can be used in the reaction are not limited to the above-described reagents.

Furthermore, in Synthesis Schemes (s1-1) to (s1-3), Synthesis Scheme (s2-1), Synthesis Scheme (s2-2), and Synthesis Scheme (s3-1), an Ullmann reaction using copper or a copper compound can also be performed. Examples of the base to be used include an inorganic base such as potassium carbonate. As the solvent that can be used in the reaction, 1,3-dimethyl-3,4,5,6-tetrahydro-2(1H) pyrimidinone (DMPU), toluene, xylene, benzene, and the like can be given. In the Ullmann reaction, when the reaction temperature is higher than or equal to 100° C., a target substance can be obtained in a shorter time in a higher yield; therefore, it is preferable to use DMPU or xylene having a high boiling point. A reaction temperature of 150° C. or higher is further preferred, and accordingly, DMPU is further preferably used. Reagents that can be used in the reaction are not limited to the above-described reagents.

The methods for synthesizing the organic compounds represented by General Formula (G1-1), General Formula (G1-2), and General Formula (G2) are described so far; however, the methods for synthesizing the organic compounds are not limited to Synthesis Scheme (s1-1) to Synthesis Scheme (s1-3), Synthesis Scheme (s2-1), Synthesis Scheme (s2-2), and Synthesis Scheme (s3-1).

This embodiment can be freely combined with any of the other embodiments and the examples.

### Embodiment 2

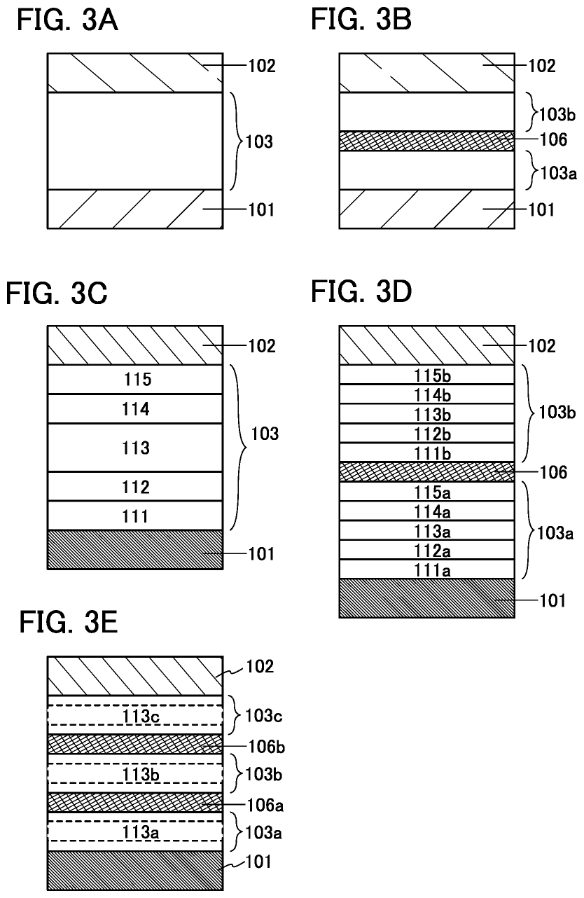

In this embodiment, structures of the light-emitting device including any of the organometallic complexes described in Embodiment 1 will be described with reference to FIGS. 3A to 3E.

### <Basic Structure of Light-Emitting Device>

A basic structure of the light-emitting device is described. FIG. 3A illustrates a light-emitting device having a structure (single structure) in which an organic EL layer including a light-emitting layer is provided between a pair of electrodes. Specifically, the organic compound layer 103 is positioned between the first electrode 101 and the second electrode 102.

FIG. 3B illustrates a light-emitting device that has a stacked-layer structure (tandem structure) in which a plurality of organic compound layers (two organic compound layers 103a and 103b in FIG. 3B) are provided between a pair of electrodes and a charge-generation layer 106 is provided between the EL layers. A light-emitting device having the tandem structure enables a light-emitting apparatus that has high efficiency without changing the amount of current.

The charge-generation layer 106 has a function of injecting electrons into one of the organic compound layers 103a and 103b and injecting holes into the other of the organic compound layers 103a and 103b when a potential difference is caused between the first electrode 101 and the second electrode 102. Thus, when voltage is applied such that the potential of the first electrode 101 is higher than that of the second electrode 102 in FIG. 3B, electrons are injected from the charge-generation layer 106 into the organic compound layer 103a and holes are injected from the charge-generation layer 106 into the organic compound layer 103b.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 or the second electrode 102.

FIG. 3C illustrates a stacked-layer structure of the organic compound layer 103 in the light-emitting device of one embodiment of the present invention. In this case, the first electrode 101 is regarded as functioning as an anode and the second electrode 102 is regarded as functioning as a cathode. The organic compound layer 103 has a structure in which the hole-injection layer 111, the hole-transport layer 112, the light-emitting layer 113, the electron-transport layer 114, and the electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above examples. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can achieve higher reliability than a single-layer structure in some cases. In the case where a plurality of EL layers are provided as in the tandem structure illustrated in FIG. 3B, the layers in each EL layer are sequentially stacked from the anode side as described above. When the first electrode 101 is the cathode and the second electrode 102 is the anode, the stacking order of the layers in the organic compound layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the organic compound layers (103, 103a, and 103b) contains an appropriate combination of a light-emitting substance and a plurality of substances, so that fluorescent light of a desired color or phosphorescent light of a desired color can be obtained. The light-emitting layer 113 may have a stacked-layer structure having different emission colors. In that case, light-emitting substances and other substances may be different between the stacked light-emitting layers. Alternatively, the plurality of organic compound layers (103a and 103b) in FIG. 3B may exhibit their respective emission colors. Also in that case, the light-emitting substances and other substances may be different between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 3C. Thus, light from the light-emitting layer 113 in the organic compound layer 103 can be resonated between the electrodes and light emitted through the second electrode 102 can be intensified. This makes it easy to achieve high definition. In addition, emission intensity with a predetermined wavelength in the front direction can be increased, leading to lower power consumption.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: k) emitted from the light-emitting layer 113, it is preferable to adjust each of the optical path length from the first electrode 101 to a region where the desired light is emitted in the light-emitting layer 113 (light-emitting region) and the optical path length from the second electrode 102 to the region where the desired light is emitted in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, the above effect can be considered to be obtained sufficiently wherever the reflective regions may be present in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, the above effect can be considered to be obtained sufficiently wherever the reflective region and the light-emitting region may be present in the first electrode 101 and the light-emitting layer that emits the desired light, respectively.

The light-emitting device illustrated in FIG. 3D is a light-emitting device having the tandem structure. The tandem structure enables a light-emitting device to emit light with high luminance. Furthermore, the amount of current needed for a predetermined luminance can be smaller in the tandem structure than in the single structure; thus, the tandem structure enables higher reliability. In addition, power consumption can be reduced.

The light-emitting device illustrated in FIG. 3E is an example of the light-emitting device having the tandem structure illustrated in FIG. 3B, and includes three organic compound layers (103a, 103b, and 103c) stacked with charge-generation layers (106a and 106b) positioned therebetween, as illustrated in FIG. 3E. The three organic compound layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light, or the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity lower than or equal to 1×10⁻² Ωcm.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity lower than or equal to 1×10⁻² Ωcm.

### <Specific Structure of Light-Emitting Device>

Next, a specific structure of the light-emitting device of one embodiment of the present invention will be described. Here, the description is made using FIG. 3D illustrating the tandem structure. Note that the structure of the EL layer also applies to the structure of the light-emitting devices having the single structure in FIGS. 3A and 3C. When the light-emitting device in FIG. 3D has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the organic compound layer 103b, with the use of a material selected as appropriate.

### <Materials of Light-Emitting Device>

### <<Light-Emitting Layer>>

The light-emitting layers (113, 113a, and 113b) contain a light-emitting substance. Note that as a light-emitting substance that can be used in the light-emitting layers (113, 113a, and 113b), a substance that exhibits emission color such as blue, violet, bluish violet, green, yellowish green, yellow, orange, or red can be used as appropriate. When a plurality of light-emitting layers are provided, the use of different light-emitting substances for the light-emitting layers enables a structure that exhibits different emission colors (e.g., white light emission obtained by a combination of complementary emission colors). Furthermore, one light-emitting layer may have a stacked-layer structure including different light-emitting substances.

The light-emitting layers (113, 113a, and 113b) may each contain one or more kinds of organic compounds (e.g., a host material) in addition to a light-emitting substance (a guest material).

Specifically, the light-emitting layer 113 can have the structure that is described in Embodiment 1 with reference to FIG. 1B. In the light-emitting layer 113, the host materials 118 are present in the largest proportion by weight, and the guest material 119 (phosphorescent compound) is dispersed in the host materials 118. The T₁ levels of the host materials 118 (the organic compound 118_1 and the organic compound 118_2) in the light-emitting layer 113 are preferably higher than the T₁ level of the guest material (the guest material 119) in the light-emitting layer 113.

As the organic compound 118_1, a material having an electron-transport property higher than a hole-transport property can be used, and a material having an electron mobility higher than or equal to 1×10⁻⁶ cm²/Vs is preferable. A compound having a π-electron deficient heteroaromatic ring skeleton such as a nitrogen-containing heteroaromatic compound, or a zinc- or aluminum-based metal complex can be used, for example, as a material which easily accepts electrons (a material having an electron-transport property). Examples of the compound having a π-electron deficient heteroaromatic ring skeleton include compounds such as an oxadiazole derivative, a triazole derivative, a benzimidazole derivative, a quinoxaline derivative, a dibenzoquinoxaline derivative, a phenanthroline derivative, a pyridine derivative, a bipyridine derivative, a pyrimidine derivative, and a triazine derivative. Examples of the zinc- or aluminum-based metal complex include a metal complex having a quinoline ligand, a metal complex having a benzoquinoline ligand, a metal complex having an oxazole ligand, and a metal complex having a thiazole ligand.

Specific examples thereof include metal complexes having a quinoline or benzoquinoline skeleton, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq). Alternatively, a metal complex having an oxazole-based or thiazole-based ligand, such as bis[2-(2-benzoxazolyl)phenolato]zinc(II) (abbreviation: ZnPBO) or bis[2-(2-benzothiazolyl)phenolato]zinc(II) (abbreviation: ZnBTZ), can be used. Other than such metal complexes, any of the following can be used: heterocyclic compounds such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 9-[4-(4,5-diphenyl-4H-1,2,4-triazol-3-yl)phenyl]-9H-carbazole (abbreviation: CzTAZ1), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), bathophenanthroline (abbreviation: BPhen), and bathocuproine (abbreviation: BCP); heterocyclic compounds having a diazine skeleton such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 2-[3-(3,9′-bi-9H-carbazol-9-yl)phenyl]dibenzo[f,h]quinoxaline (abbreviation: 2mCzCzPDBq), 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), and 4,6-bis[3-(9H-carbazol-9-yl)phenyl]pyrimidine (abbreviation: 4,6mCzP2Pm); heterocyclic compounds having a triazine skeleton such as 2-{4-[3-(N-phenyl-9H-carbazol-3-yl)-9H-carbazol-9-yl]phenyl}-4,6-diphenyl-1,3,5-triazine (abbreviation: PCCzPTzn); heterocyclic compounds having a pyridine skeleton such as 3,5-bis[3-(9H-carbazol-9-yl)phenyl]pyridine (abbreviation: 35DCzPPy) and 1,3,5-tri[3-(3-pyridyl)phenyl]benzene (abbreviation: TmPyPB); and heteroaromatic compounds such as 4,4′-bis(5-methylbenzoxazol-2-yl)stilbene (abbreviation: BzOs). Among the heterocyclic compounds, the heterocyclic compounds having a triazine skeleton, a diazine (pyrimidine, pyrazine, or pyridazine) skeleton, or a pyridine skeleton are highly reliable and stable and are thus preferably used. In addition, the heterocyclic compounds having any of these skeletons have a high electron-transport property to contribute to a reduction in driving voltage. Further alternatively, a high-molecular compound such as poly(2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy) can be used. The substances described here are mainly substances having an electron mobility higher than or equal to 1×10⁻⁶ cm²/Vs. Note that other substances may also be used as long as their electron-transport properties are higher than their hole-transport properties.

As the organic compound 118_1, any of the organic compounds represented by Structural Formula (400) to Structural Formula (415) can be used, for example.

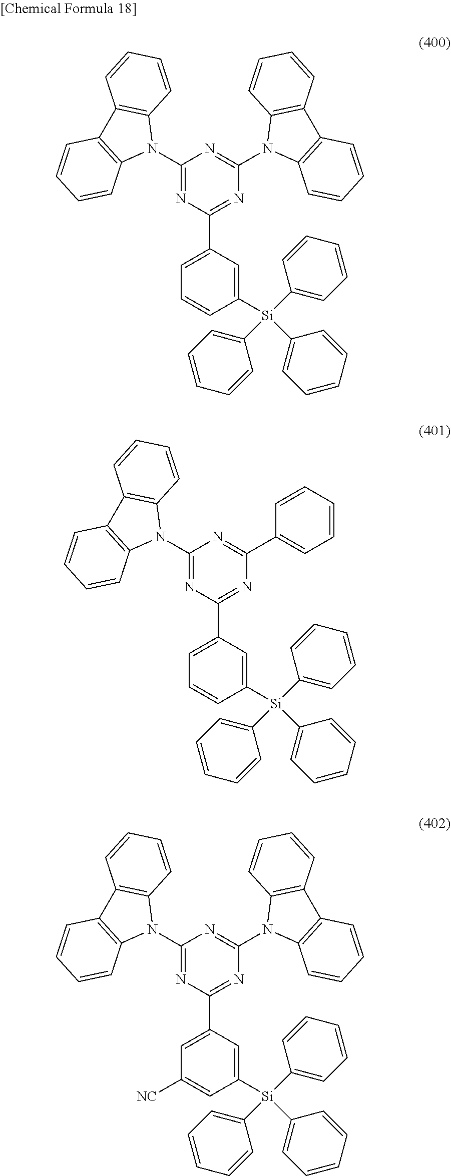 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc(-c2nc(-c3cccc([Si](c4ccccc4)(c4ccccc4)c4ccccc4)c3)nc(-n3c4ccccc4c4ccccc43)n2)cc1 ; N#Cc1cc(-c2nc(-n3c4ccccc4c4ccccc43)nc(-n3c4ccccc4c4ccccc43)n2)cc([Si](c2ccccc2)(c2ccccc2)c2ccccc2)c1]

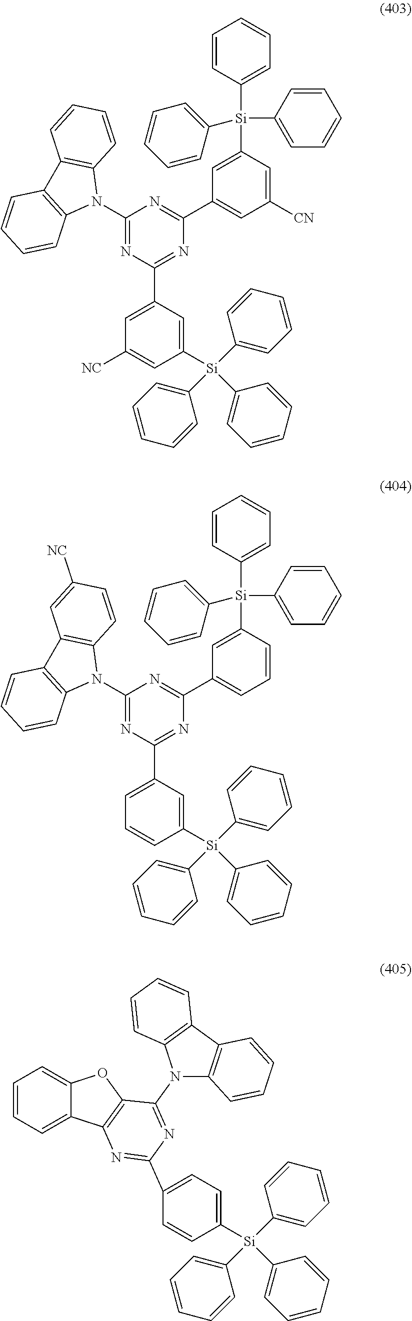 [SMILES: N#Cc1cc(-c2nc(-c3cc(C#N)cc([Si](c4ccccc4)(c4ccccc4)c4ccccc4)c3)nc(-n3c4ccccc4c4ccccc43)n2)cc([Si](c2ccccc2)(c2ccccc2)c2ccccc2)c1 ; N#Cc1ccc2c(c1)c1ccccc1n2-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; c1ccc([Si](c2ccccc2)(c2ccccc2)c2ccc(-c3nc(-n4c5ccccc5c5ccccc54)c4oc5ccccc5c4n3)cc2)cc1]

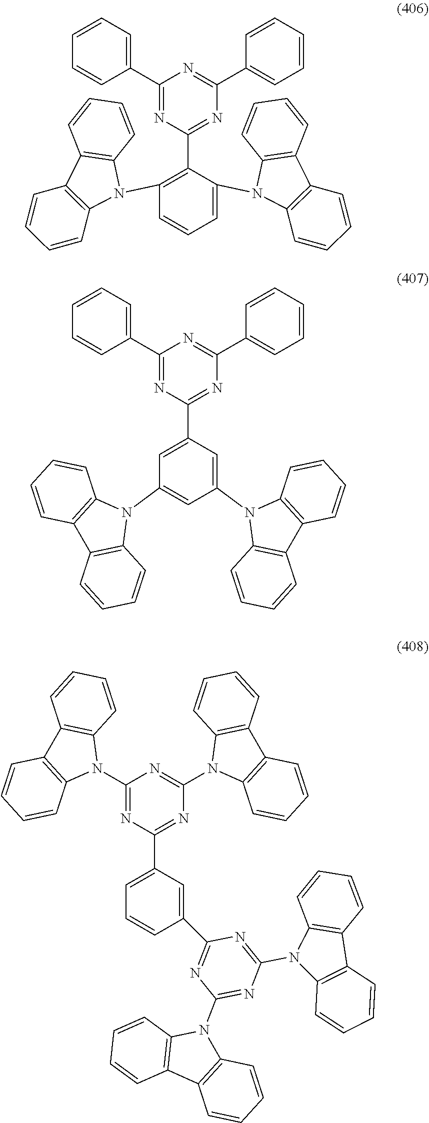 [SMILES: c1ccc(-c2nc(-c3ccccc3)nc(-c3c(-n4c5ccccc5c5ccccc54)cccc3-n3c4ccccc4c4ccccc43)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2nc(-n3c4ccccc4c4ccccc43)nc(-n3c4ccccc4c4ccccc43)n2)cc(-c2nc(-n3c4ccccc4c4ccccc43)nc(-n3c4ccccc4c4ccccc43)n2)c1]

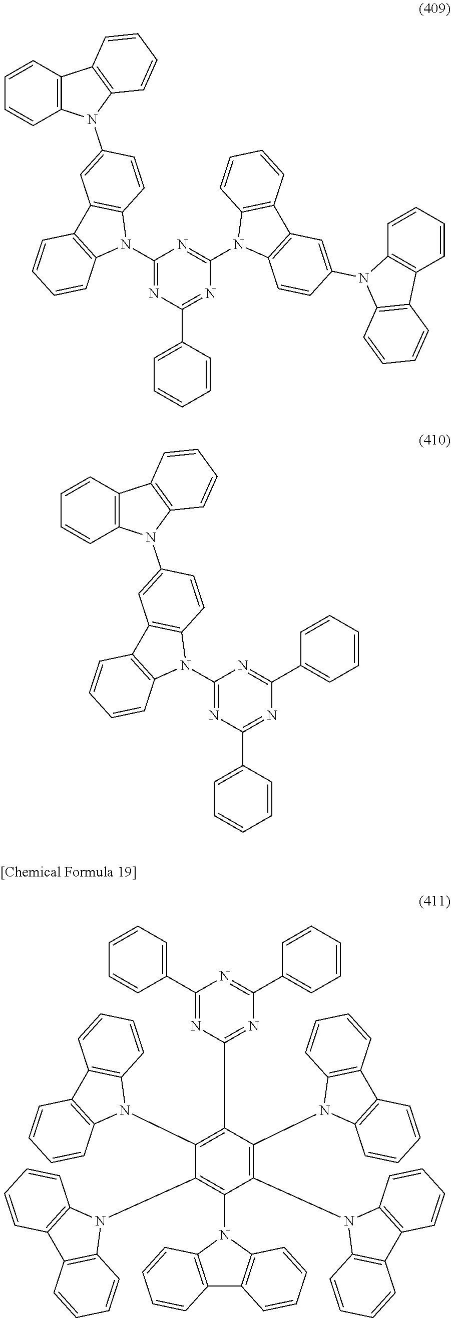

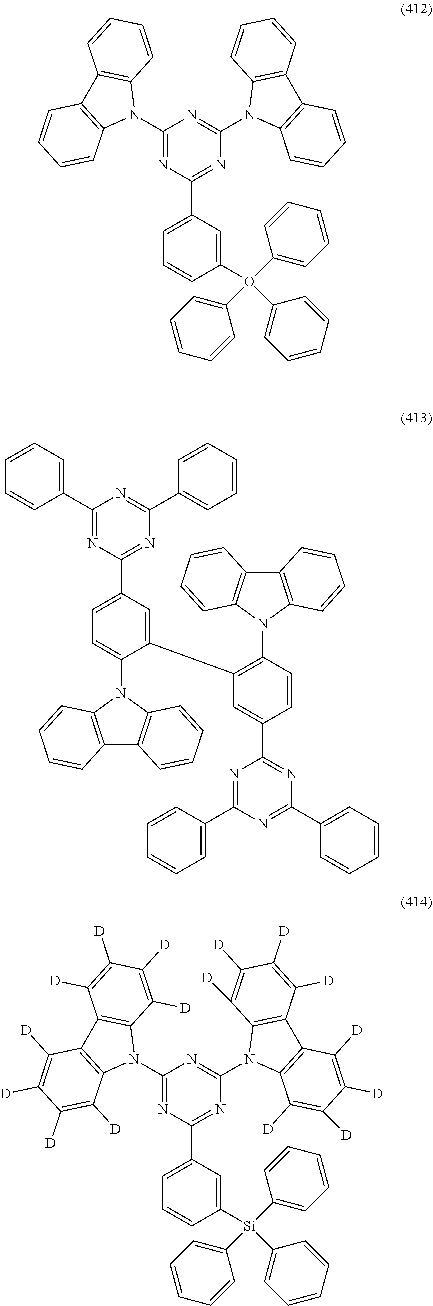

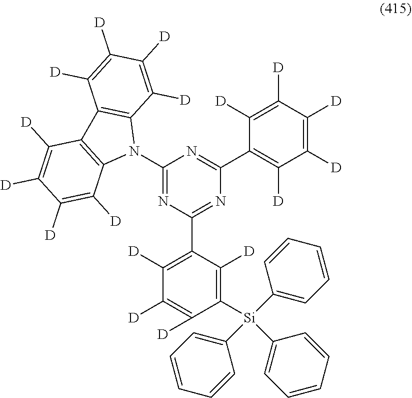 [SMILES: [2H]c1c([2H])c([2H])c(-c2nc(-c3c([2H])c([2H])c([2H])c([Si](c4ccccc4)(c4ccccc4)c4ccccc4)c3[2H])nc(-n3c4c([2H])c([2H])c([2H])c([2H])c4c4c([2H])c([2H])c([2H])c([2H])c43)n2)c([2H])c1[2H]]

As the organic compound 118_2, a substance which can form an exciplex together with the organic compound 118_1 is preferably used. Specifically, the organic compound 118_2 preferably includes a skeleton having a high donor property, such as a π-electron rich heteroaromatic ring skeleton or an aromatic amine skeleton. Examples of the compound having a π-electron rich heteroaromatic ring skeleton include heteroaromatic compounds such as a dibenzothiophene derivative, a dibenzofuran derivative, and a carbazole derivative. In that case, it is preferable that the organic compound 118_1, the organic compound 118_2, and the guest material 119 (phosphorescent compound) be selected such that the emission peak of the exciplex formed by the organic compounds 118_1 and 118_2 overlaps with an absorption band, specifically the longest-wavelength absorption band, of a triplet metal to ligand charge transfer (MLCT) transition of the guest material 119 (phosphorescent compound). This makes it possible to provide a light-emitting device with drastically improved emission efficiency. Note that in the case where a thermally activated delayed fluorescence material is used instead of the phosphorescent compound, it is preferable that the longest-wavelength absorption band be a singlet absorption band.

As the organic compound 118_2, any of the hole-transport materials given below can be used.

A material having a hole-transport property higher than an electron-transport property can be used as a hole-transport material, and a material having a hole mobility higher than or equal to 1×10⁻⁶ cm²/Vs is preferable. Specifically, an aromatic amine, a carbazole derivative, an aromatic hydrocarbon, a stilbene derivative, or the like can be used. Furthermore, the hole-transport material may be a high molecular compound.

Examples of the aromatic amine compounds that can be used as the material having a high hole-transport property are N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), N,N-bis[4-bis(3-methylphenyl)aminophenyl]-N,N-diphenyl-4,4′-diaminobiphenyl (abbreviation: DNTPD), 1,3,5-tris[N-(4-diphenylaminophenyl)-N-phenylamino]benzene (abbreviation: DPA3B), and the like.

Specific examples of the carbazole derivative include 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples of the carbazole derivative are 4,4′-di(9H-carbazol-9-yl)biphenyl (abbreviation: CBP), 1,3,5-tris[4-(N-carbazolyl)phenyl]benzene (abbreviation: TCPB), 9-[4-(10-phenyl-9-anthracenyl)phenyl]-9H-carbazole (abbreviation: CzPA), and 1,4-bis[4-(N-carbazolyl)phenyl]-2,3,5,6-tetraphenylbenzene.

Examples of the aromatic hydrocarbon include 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, and 2,5,8,11-tetra(tert-butyl)perylene. Other examples include pentacene and coronene. The aromatic hydrocarbon having a hole mobility higher than or equal to 1×10⁻⁶ cm²/Vs and having 14 to 42 carbon atoms is particularly preferable.

The aromatic hydrocarbon may have a vinyl skeleton. Examples of the aromatic hydrocarbon having a vinyl skeleton include 4,4′-bis(2,2-diphenylvinyl)biphenyl (abbreviation: DPVBi) and 9,10-bis[4-(2,2-diphenylvinyl)phenyl]anthracene (abbreviation: DPVPA).

A high molecular compound such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N-phenylamino}phenyl)methacrylamide] (abbreviation: PTPDMA), or poly[N,N-bis(4-butylphenyl)-N,N-bis(phenyl)benzidine] (abbreviation: Poly-TPD) can also be used.

Examples of the material having a high hole-transport property include aromatic amine compounds such as 4,4′-bis[N-(1-naphthyl)-N-phenylamino]biphenyl (abbreviation: NPB or α-NPD), N,N-diphenyl-N,N-bis(3-methylphenyl)-4,4′-diaminobiphenyl (abbreviation: TPD), 4,4′,4″-tris(carbazol-9-yl)triphenylamine (abbreviation: TCTA), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), N-(9,9-spirobi[9H-fluoren]-2-yl)-N,N,N-triphenyl-1,4-phenylenediamine (abbreviation: DPASF), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N-bis(9-phenylcarbazol-3-yl)-N,N′-diphenylbenzene-1,3-diamine (abbreviation: PCA2B), N,N,N′-triphenyl-N,N,N′-tris(9-phenylcarbazol-3-yl)benzene-1,3,5-triamine (abbreviation: PCA3B), N-(9,9-diphenyl-9H-fluoren-2-yl)-N,9-diphenyl-9H-carbazol-3-amine (abbreviation: PCAFLP(2)), N-(9,9-diphenyl-9H-fluoren-2-yl)-N,9-diphenyl-9H-carbazol-2-amine (abbreviation: PCAFLP(2)-02), N-(4-biphenyl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), N-(9,9-spirobi[9H-fluoren]-2-yl)-N,9-diphenylcarbazol-3-amine (abbreviation: PCASF), N,N-diphenyl-N,N-bis(4-diphenylaminophenyl)spirobi[9H-fluorene]-2,7-diamine (abbreviation: DPA2SF), N-[4-(9H-carbazol-9-yl)phenyl]-N-(4-phenyl)phenylaniline (abbreviation: YGA1BP), and N,N-bis[4-(carbazol-9-yl)phenyl]-N,N-diphenyl-9,9-dimethylfluorene-2,7-diamine (abbreviation: YGA2F). Other examples are amine compounds, carbazole compounds, thiophene compounds, furan compounds, fluorene compounds, triphenylene compounds, phenanthrene compounds, and the like such as 3-[4-(1-naphthyl)phenyl]-9-phenyl-9H-carbazole (abbreviation: PCPN), 9-[4-(9-phenyl-9H-carbazol-3-yl)phenyl]phenanthrene (abbreviation: PCPPn), 3,3′-bis(9-phenyl-9H-carbazole) (abbreviation: PCCP), 1,3-bis(N-carbazolyl)benzene (abbreviation: mCP), 3,6-bis(3,5-diphenylphenyl)-9-phenylcarbazole (abbreviation: CzTP), 9′-phenyl-9′H-9,3′:6′,9″-tercarbazole (abbreviation: PhCzGI), 2,8-di(9H-carbazol-9-yl)dibenzothiophene (abbreviation: Cz2DBT), 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Among the above compounds, compounds having a pyrrole skeleton, a furan skeleton, a thiophene skeleton, or an aromatic amine skeleton, which have high stability and high reliability, are preferable. In addition, the compounds having any of these skeletons have a high hole-transport property to contribute to a reduction in driving voltage.

The guest material 119 (phosphorescent compound) can be an iridium-, rhodium-, or platinum-based organometallic complex or metal complex. A particularly preferable example of the metal complex is a platinum complex. Other examples include a platinum complex having a nitrogen-containing heterocyclic carbene. An organoiridium complex such as an iridium-based orthometalated complex may be used. As an orthometalated ligand, a 4H-triazole ligand, a 1H-triazole ligand, an imidazole ligand, a pyridine ligand, a pyrimidine ligand, a pyrazine ligand, an isoquinoline ligand, or the like can be used.

The organic compound 118_1, the organic compound 118_2, and the guest material 119 (phosphorescent compound) are preferably selected such that the LUMO (Lowest Unoccupied Molecular Orbital) level of the guest material 119 (phosphorescent compound) is higher than that of the organic compound 118_1 and the HOMO (Highest Occupied Molecular Orbital) level of the guest material 119 is lower than that of the organic compound 118_2. With this structure, a light-emitting device with high emission efficiency and low driving voltage can be obtained.

The organic compound 118_1, the organic compound 118_2, and the guest material 119 (phosphorescent compound) are preferably selected such that the LUMO level of the guest material 119 (phosphorescent compound) is higher than that of the organic compound 118_1 and the HOMO level of the guest material 119 is higher than that of the organic compound 118_2. With this structure, a light-emitting device with high emission efficiency and low driving voltage can be obtained.

The organic compound 118_1 and the guest material 119 (phosphorescent compound) are preferably selected such that the energy difference between the LUMO level of the organic compound 118_1 and the HOMO level of the guest material 119 (phosphorescent compound) is greater than or equal to the energy that is calculated from the longest-wavelength absorption edge in the absorption spectrum of the guest material 119 (phosphorescent compound). With this structure, a light-emitting device with high emission efficiency and low driving voltage can be obtained.

The longest-wavelength absorption edge in an absorption spectrum can be determined from a Tauc plot, with an assumption of direct transition, of a measured absorption spectrum of a target substance in the form of a thin film or a thin film in which a matrix material is doped with the target substance. Alternatively, an absorption spectrum of the target substance in a solution may be measured and an absorption edge may be calculated from the intersection of the horizontal axis (wavelength) or the base line and a tangent drawn at the half of a peak value on the longer wavelength side in the longest-wavelength peak or shoulder peak in the absorption spectrum. There is no particular limitation on a solvent of the solution; a solvent with relatively low polarity, such as toluene or chloroform, is preferable.

The values of HOMO and LUMO levels used in this specification can be obtained by electrochemical measurement. Typical examples of the electrochemical measurement include cyclic voltammetry (CV) measurement and differential pulse voltammetry (DPV) measurement.

Specifically, in the cyclic voltammetry (CV) measurement, the values (E) of HOMO and LUMO levels can be calculated on the basis of an oxidation peak potential (Eₚₐ) and a reduction peak potential (E_{pc}), which are obtained by changing the potential of a working electrode with respect to a reference electrode. In the measurement, a HOMO level and a LUMO level are obtained by potential scanning in positive direction and potential scanning in negative direction, respectively. The scanning speed in the measurement is 0.1 V/s.

Calculation steps of the HOMO level and the LUMO level are described in detail. A standard oxidation-reduction potential (Eₒ) (=Eₚₐ+E_{pc})/2) is calculated from an oxidation peak potential (Eₚₐ) and a reduction peak potential (E_{pc}), which are obtained by the cyclic voltammogram of a material. Then, the standard oxidation-reduction potential (Eₒ) is subtracted from the potential energy (Eₓ) of the reference electrode with respect to a vacuum level, whereby each of the values (E) (=Eₓ−Eₒ) of HOMO and LUMO levels can be obtained.

Note that the reversible oxidation-reduction wave is obtained in the above case; in the case where an irreversible oxidation-reduction wave is obtained, the HOMO level is calculated as follows: a value obtained by subtracting a predetermined value (0.1 eV) from an oxidation peak potential (Eₚₐ) is assumed to be a reduction peak potential (E_{pc}), and a standard oxidation-reduction potential (Eₒ) is calculated to one decimal place. To calculate the LUMO level, a value obtained by adding a predetermined value (0.1 eV) to a reduction peak potential (E_{pc}) is assumed to be an oxidation peak potential (Eₚₐ), and a standard oxidation-reduction potential (Eₒ) is calculated to one decimal place.

Examples of a substance that has an emission peak in the blue or green wavelength range include organometallic iridium complexes having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: Ir(mpptz-dmp)₃), tris(5-methyl-3,4-diphenyl-4H-1,2,4-triazolato)iridium(III) (abbreviation: Ir(Mptz)₃), tris[4-(3-biphenyl)-5-isopropyl-3-phenyl-4H-1,2,4-triazolato]iridium(III) (abbreviation: Ir(iPrptz-3b)₃), and tris[3-(5-biphenyl)-5-isopropyl-4-phenyl-4H-1,2,4-triazolato]iridium(III) (abbreviation: Ir(iPr5btz)₃); organometallic iridium complexes having a 1H-triazole skeleton, such as tris[3-methyl-1-(2-methylphenyl)-5-phenyl-1H-1,2,4-triazolato]iridium(III) (abbreviation: Ir(Mptz1-mp)₃) and tris(1-methyl-5-phenyl-3-propyl-1H-1,2,4-triazolato)iridium(III) (abbreviation: Ir(Prptzl-Me)₃); organometallic iridium complexes having an imidazole skeleton, such as fac-tris[1-(2,6-diisopropylphenyl)-2-phenyl-1H-imidazole]iridium(III) (abbreviation: Ir(iPrpim)₃) and tris[3-(2,6-dimethylphenyl)-7-methylimidazo[1,2-J]phenanthridinato]iridium(III) (abbreviation: Ir(dmpimpt-Me)₃); and organometallic iridium complexes in which a phenylpyridine derivative having an electron-withdrawing group is a ligand, such as bis[2-(4′,6′-difluorophenyl)pyridinato-N,C²′]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: Ir(CF₃ppy)₂(pic)), and bis[2-(4′,6′-difluorophenyl)pyridinato-N,C^{2′}]iridium(III) acetylacetonate (abbreviation: FIr(acac)). Among the materials listed above, the organometallic iridium complexes having a nitrogen-containing five-membered heterocyclic skeleton, such as a 4H-triazole skeleton, a 1H-triazole skeleton, or an imidazole skeleton, which have high triplet excitation energy as well as high reliability or high emission efficiency, are particularly preferable.

Examples of a substance that has an emission peak in the green or yellow wavelength range include organometallic iridium complexes having a pyrimidine skeleton, such as tris(4-methyl-6-phenylpyrimidinato)iridium(III) (abbreviation: Ir(mppm)₃), tris(4-t-butyl-6-phenylpyrimidinato)iridium(III) (abbreviation: Ir(tBuppm)₃), (acetylacetonato)bis(6-methyl-4-phenylpyrimidinato)iridium(III) (abbreviation: Ir(mppm)₂(acac)), (acetylacetonato)bis(6-tert-butyl-4-phenylpyrimidinato)iridium(III) (abbreviation: Ir(tBuppm)₂(acac)), (acetylacetonato)bis[4-(2-norbornyl)-6-phenylpyrimidinato]iridium(III) (abbreviation: Ir(nbppm)₂(acac)), (acetylacetonato)bis[5-methyl-6-(2-methylphenyl)-4-phenylpyrimidinato]iridium(III) (abbreviation: Ir(mpmppm)₂(acac)), (acetylacetonato)bis{4,6-dimethyl-2-[6-(2,6-dimethylphenyl)-4-pyrimidinyl-κN³]phenyl-κC}iridium(III) (abbreviation: Ir(dmppm-dmp)₂(acac)), and (acetylacetonato)bis(4,6-diphenylpyrimidinato)iridium(III) (abbreviation: Ir(dppm)₂(acac)); organometallic iridium complexes having a pyrazine skeleton, such as (acetylacetonato)bis(3,5-dimethyl-2-phenylpyrazinato)iridium(III) (abbreviation: Ir(mppr-Me)₂(acac)) and (acetylacetonato)bis(5-isopropyl-3-methyl-2-phenylpyrazinato)iridium(III) (abbreviation: Ir(mppr-iPr)₂(acac)); organometallic iridium complexes having a pyridine skeleton, such as tris(2-phenylpyridinato-N,C²′)iridium(III) (abbreviation: Ir(ppy)₃), bis(2-phenylpyridinato-N,C^{2′})iridium(III) acetylacetonate (abbreviation: Ir(ppy)₂(acac)), bis(benzo[h]quinolinato)iridium(III) acetylacetonate (abbreviation: Ir(bzq)₂(acac)), tris(benzo[h]quinolinato)iridium(III) (abbreviation: Ir(bzq)₃), tris(2-phenylquinolinato-N,C^{2′})iridium(III) (abbreviation: Ir(pq)₃), and bis(2-phenylquinolinato-N,C^{2′})iridium(III) acetylacetonate (abbreviation: Ir(pq)₂(acac)); organometallic iridium complexes such as bis(2,4-diphenyl-1,3-oxazolato-N,C^{2′})iridium(III) acetylacetonate (abbreviation: Ir(dpo)₂(acac)), bis{2-[4′-(perfluorophenyl)phenyl]pyridinato-N,C^{2′}}iridium(III) acetylacetonate (abbreviation: Ir(p-PF-ph)₂(acac)), and bis(2-phenylbenzothiazolato-N,C^{2′})iridium(III) acetylacetonate (abbreviation: Ir(bt)₂(acac)); and a rare earth metal complex such as tris(acetylacetonato)(monophenanthroline)terbium(III) (abbreviation: Tb(acac)₃(Phen)). Among the materials listed above, the organometallic iridium complexes having a pyrimidine skeleton, which have distinctively high reliability or emission efficiency, are particularly preferable.

Examples of a substance that has an emission peak in the yellow or red wavelength range include organometallic iridium complexes having a pyrimidine skeleton, such as (diisobutyrylmethanato)bis[4,6-bis(3-methylphenyl)pyrimidinato]iridium(III) (abbreviation: Ir(5mdppm)₂(dibm)), bis[4,6-bis(3-methylphenyl)pyrimidinato](dipivaloylmethanato)iridium(III) (abbreviation: Ir(5mdppm)₂(dpm)), and bis[4,6-di(naphthalen-1-yl)pyrimidinato](dipivaloylmethanato)iridium(III) (abbreviation: Ir(d1npm)₂(dpm)); organometallic iridium complexes having a pyrazine skeleton, such as (acetylacetonato)bis(2,3,5-triphenylpyrazinato)iridium(III) (abbreviation: Ir(tppr)₂(acac)), bis(2,3,5-triphenylpyrazinato)(dipivaloylmethanato)iridium(III) (abbreviation: Ir(tppr)₂(dpm)), and (acetylacetonato)bis[2,3-bis(4-fluorophenyl)quinoxalinato]iridium(III) (abbreviation: Ir(Fdpq)₂(acac)); organometallic iridium complexes having a pyridine skeleton, such as tris(1-phenylisoquinolinato-N,C²′)iridium(III) (abbreviation: Ir(piq)₃) and bis(1-phenylisoquinolinato-N,C²′)iridium(III) acetylacetonate (abbreviation: Ir(piq)₂(acac)); a platinum complex such as 2,3,7,8,12,13,17,18-octaethyl-21H,23H-porphyrin platinum(II) (abbreviation: PtOEP); and rare earth metal complexes such as tris(1,3-diphenyl-1,3-propanedionato)(monophenanthroline)europium(III) (abbreviation: Eu(DBM)₃(Phen)) and tris[1-(2-thenoyl)-3,3,3-trifluoroacetonato](monophenanthroline)europium(III) (abbreviation: Eu(TTA)₃(Phen)). Among the materials listed above, the organometallic iridium complexes having a pyrimidine skeleton, which have distinctively high reliability or emission efficiency, are particularly preferable. Furthermore, the organometallic iridium complexes having a pyrazine skeleton can provide red light emission with favorable chromaticity.

The light-emitting substance included in the light-emitting layer 113 is a material that can convert the triplet excitation energy into light emission. As an example of the material that can convert the triplet excitation energy into light emission, a thermally activated delayed fluorescence (TADF) material can be given in addition to phosphorescent compounds. Therefore, it is acceptable that the “phosphorescent compound” in the description is replaced with the “thermally activated delayed fluorescence material”. Note that the thermally activated delayed fluorescence material has a small difference between the triplet excitation energy level and the singlet excitation energy level and a function of converting triplet excitation energy into singlet excitation energy by reverse intersystem crossing. Thus, the thermally activated delayed fluorescence material can up-convert a triplet excited state into a singlet excited state (i.e., reverse intersystem crossing is possible) using a little thermal energy and efficiently exhibit light emission (fluorescence) from the singlet excited state. The thermally activated delayed fluorescence is efficiently obtained under the condition where the difference in energy between the triplet excitation energy level and the singlet excitation energy level is preferably larger than 0 eV and smaller than or equal to 0.2 eV, further preferably larger than 0 eV and smaller than or equal to 0.1 eV.

In the case where the thermally activated delayed fluorescence material is composed of one kind of material, any of the following materials can be used, for example.

First, a derivative of a fullerene or the like, an acridine derivative such as proflavine, eosin, and the like can be given as examples. Other examples include a metal-containing porphyrin, such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP).

As the thermally activated delayed fluorescence material composed of one kind of material, a heterocyclic compound having a π-electron rich heteroaromatic ring and a π-electron deficient heteroaromatic ring can also be used. Specific examples include 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), and 10-phenyl-10H,10′H-spiro[acridin-9,9′-anthracen]-10′-one (abbreviation: ACRSA). The heterocyclic compound is preferable because of its high electron-transport and hole-transport properties due to the π-electron rich heteroaromatic ring and the π-electron deficient heteroaromatic ring contained therein. Among skeletons having the π-electron deficient heteroaromatic ring, a diazine skeleton (a pyrimidine skeleton, a pyrazine skeleton, or a pyridazine skeleton) and a triazine skeleton are particularly preferable because of their high stability and high reliability. Among skeletons having the π-electron rich heteroaromatic ring, an acridine skeleton, a phenoxazine skeleton, a thiophene skeleton, a furan skeleton, and a pyrrole skeleton have high stability and high reliability; therefore, one or more of these skeletons are preferably included. As the pyrrole skeleton, an indole skeleton, a carbazole skeleton, or a 3-(9-phenyl-9H-carbazol-3-yl)-9H-carbazole skeleton is particularly preferable. It is particularly preferable that the π-electron rich heteroaromatic ring be directly bonded to the π-electron deficient heteroaromatic ring, in which case the donor property of the π-electron rich heteroaromatic ring and the acceptor property of the π-electron deficient heteroaromatic ring are both increased and the difference between the singlet excitation energy level and the triplet excitation energy level becomes small.

As a light-emitting substance, for example, any of the organic compounds represented by Structural Formulas (500) to (511) can be used.

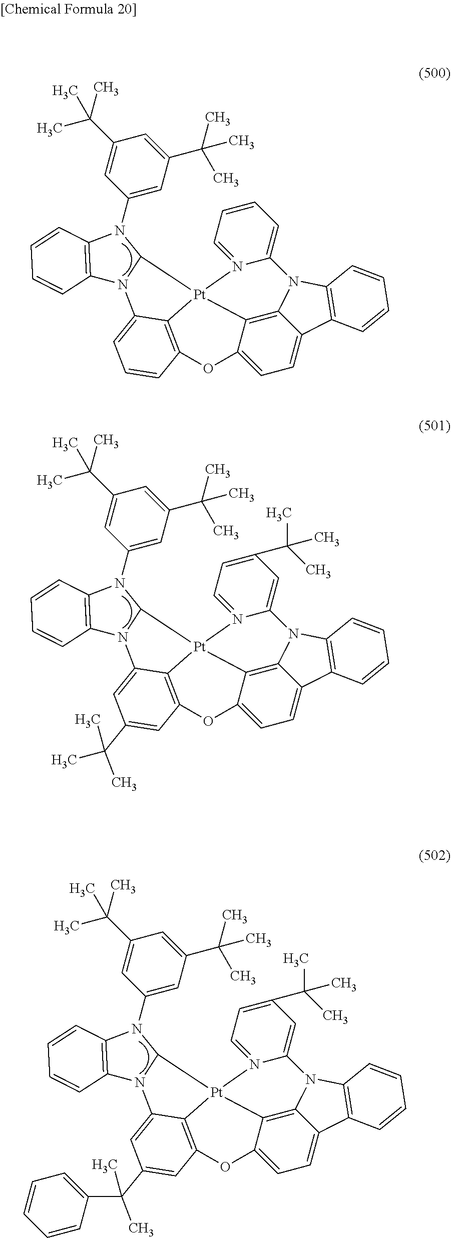

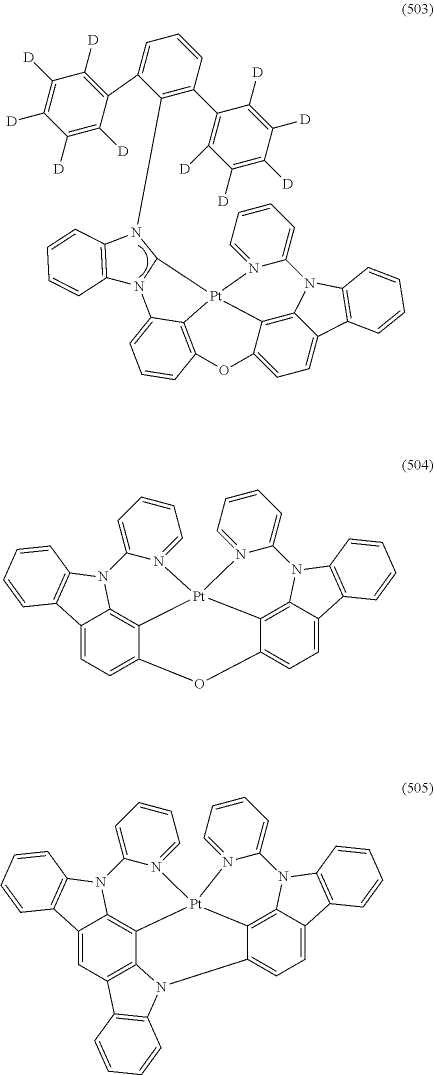

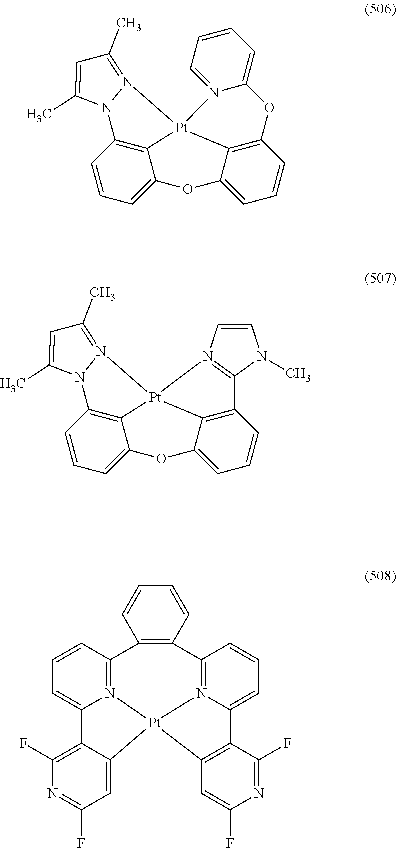

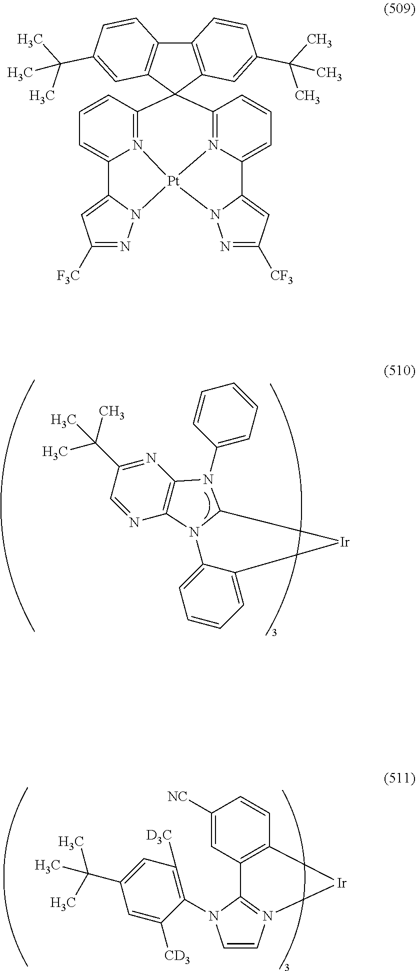

The light-emitting layer 113 can include two or more layers. For example, in the case where the light-emitting layer 113 is formed by stacking a first light-emitting layer and a second light-emitting layer in this order from the hole-transport layer side, the first light-emitting layer is formed using a substance having a hole-transport property as the host material and the second light-emitting layer is formed using a substance having an electron-transport property as the host material. Alight-emitting substance included in the first light-emitting layer may be the same as or different from a light-emitting substance included in the second light-emitting layer. In addition, the materials may have functions of emitting light of the same color or light of different colors. When light-emitting substances having functions of emitting light of different colors are used for the two light-emitting layers, light of a plurality of emission colors can be obtained at the same time. It is particularly preferable to select light-emitting substances of the light-emitting layers so that white light can be obtained by combining light emission from the two light-emitting layers.

The light-emitting layer 113 may include a material other than the host material 118 and the guest material 119.

Note that the light-emitting layer 113 can be formed by an evaporation method (including a vacuum evaporation method), an ink-jet method, a coating method, gravure printing, or the like. Besides the above-mentioned materials, an inorganic compound such as a quantum dot or a high molecular compound (e.g., an oligomer, a dendrimer, or a polymer) may be used.

### <<Hole-Injection Layer>>

The hole-injection layers (111, 111a, and 111b) inject holes from the first electrode 101 serving as the anode and the charge-generation layers (106, 106a, and 106b) to the organic compound layers (103, 103a, and 103b) and contain an organic acceptor material or a material having a high hole-injection property.

The hole-injection layers (111, 111a, and 111b) have a function of lowering a barrier for hole injection from one of the pair of electrodes (the first electrode 101 or the second electrode 102) to promote hole injection and is formed using a transition metal oxide, a phthalocyanine derivative, or an aromatic amine, for example. As examples of the transition metal oxide, molybdenum oxide, vanadium oxide, ruthenium oxide, tungsten oxide, manganese oxide, and the like can be given. As examples of the phthalocyanine derivative, phthalocyanine, metal phthalocyanine, and the like can be given. As examples of the aromatic amine, a benzidine derivative, a phenylenediamine derivative, and the like can be given. It is also possible to use a high molecular compound such as polythiophene or polyaniline; a typical example thereof is poly(ethylenedioxythiophene)/poly(styrenesulfonic acid), which is self-doped polythiophene.

As each of the hole-injection layers (111, 111a, and 111b), a layer containing a composite material of a hole-transport material and a material having a property of accepting electrons from the hole-transport material can also be used. Alternatively, a stack of a layer containing a material having an electron-accepting property and a layer containing a hole-transport material may also be used. In a steady state or in the presence of an electric field, electric charge can be transferred between these materials. As examples of the material having an electron-accepting property, organic acceptors such as a quinodimethane derivative, a chloranil derivative, and a hexaazatriphenylene derivative can be given. A specific example is a compound having an electron-withdrawing group (a halogen group or a cyano group), such as 7,7,8,8-tetracyano-2,3,5,6-tetrafluoroquinodimethane (abbreviation: F₄-TCNQ), chloranil, or 2,3,6,7,10,11-hexacyano-1,4,5,8,9,12-hexaazatriphenylene (abbreviation: HAT-CN). Alternatively, a transition metal oxide such as an oxide of a metal from Group 4 to Group 8 can be used. Specifically, vanadium oxide, niobium oxide, tantalum oxide, chromium oxide, molybdenum oxide, tungsten oxide, manganese oxide, rhenium oxide, or the like can be used. In particular, molybdenum oxide is preferable because it is stable in the air, has a low hygroscopic property, and is easily handled.

A material having a hole-transport property higher than an electron-transport property can be used as the hole-transport material, and a material having a hole mobility higher than or equal to 1×10⁻⁶ cm²/Vs is preferable. Specifically, any of the aromatic amines, carbazole derivatives, aromatic hydrocarbon, stilbene derivatives, and the like described as examples of the hole-transport material that can be used in the light-emitting layer 113 can be used. Furthermore, the hole-transport material may be a high molecular compound.

### <<Hole-Transport Layer>>

The hole-transport layers (112, 112a, and 112b) contain a hole-transport material and can be formed using any of the hole-transport materials given as examples of the material of the hole-injection layers (111, 111a, and 111b). The HOMO level of the hole-transport layers (112, 112a, and 112b) is preferably equal or close to the HOMO level of the hole-injection layers (111, 111a, and 111b) so that the hole-transport layers (112, 112a, and 112b) can have a function of transporting holes injected into the hole-injection layers (111, 111a, and 111b) to the light-emitting layers (113, 113a, and 113b).

As the hole-transport material, a substance having a hole mobility higher than or equal to 1×10⁻⁶ cm²/Vs is preferably used. Note that other substances may also be used as long as their hole-transport properties are higher than their electron-transport properties. The layer including a substance having a high hole-transport property is not limited to a single layer and may be a stack of two or more layers each containing any of the above substances.

### <<Electron-Transport Layer>>

The electron-transport layers (114, 114a, and 114b) have a function of transporting, to the light-emitting layer 113, electrons injected from the other of the pair of electrodes (the first electrode 101 or the second electrode 102) through the electron-injection layers (115, 115a, and 115b). As the electron-transport material, a material having an electron-transport property higher than a hole-transport property can be used, and a material having an electron mobility higher than or equal to 1×10⁻⁶ cm²/Vs is preferable. A compound having a π-electron deficient heteroaromatic ring skeleton such as a nitrogen-containing heteroaromatic compound or a metal complex can be used, for example, as a compound which easily accepts electrons (a material having an electron-transport property). Specific examples include a metal complex having a quinoline ligand, a benzoquinoline ligand, an oxazole ligand, or a thiazole ligand, which is described as the electron-transport material usable for the light-emitting layer 113. In addition, an oxadiazole derivative, a triazole derivative, a benzimidazole derivative, a quinoxaline derivative, a dibenzoquinoxaline derivative, a phenanthroline derivative, a pyridine derivative, a bipyridine derivative, a pyrimidine derivative, a triazine derivative, or the like can be used. A substance having an electron mobility higher than or equal to 1×10⁻⁶ cm²/Vs is preferably used. Note that other substances may also be used for the electron-transport layer as long as their electron-transport properties are higher than their hole-transport properties. Each of the electron-transport layers (114, 114a, and 114b) is not limited to a single layer and may be a stack of two or more layers each containing any of the above substances.

Between the electron-transport layer (114, 114a, or 114b) and the light-emitting layer (113, 113a, or 113b), a layer that controls transfer of electron carriers may be provided. This is a layer formed by addition of a small amount of a substance having a high electron-trapping property to a material having a high electron-transport property as described above, and the layer is capable of adjusting carrier balance by suppressing transport of electron carriers. Such a structure is very effective in inhibiting a problem (such as a reduction in element lifetime) caused when electrons pass through the light-emitting layer.

### <<Electron-Injection Layer>>

The electron-injection layers (115, 115a, and 115b) have a function of reducing a barrier for electron injection from the second electrode 102 to promote electron injection and can be formed using a Group 1 metal or a Group 2 metal, or an oxide, a halide, or a carbonate of any of these metals, for example. Alternatively, a composite material containing an electron-transport material (described above) and a material having a property of donating electrons to the electron-transport material can also be used. As examples of the material having an electron-donating property, a Group 1 metal, a Group 2 metal, an oxide of any of these metals, and the like can be given. Specifically, an alkali metal, an alkaline earth metal, or a compound thereof, such as lithium fluoride (LiF), sodium fluoride (NaF), cesium fluoride (CsF), calcium fluoride (CaF₂), or lithium oxide (LiOₓ), can be used. Alternatively, a rare earth metal compound like erbium fluoride (ErF₃) can be used. Electride may also be used for the electron-injection layer 115. Examples of the electride include a substance in which electrons are added at high concentration to calcium oxide-aluminum oxide. The electron-injection layers (115, 115a, and 115b) can be formed using the substance that can be used for the electron-transport layers (114, 114a, and 114b).

A composite material in which an organic compound and an electron donor (donor) are mixed may also be used for the electron-injection layers (115, 115a, and 115b). Such a composite material is excellent in an electron-injection property and an electron-transport property because electrons are generated in the organic compound by the electron donor. In this case, the organic compound is preferably a material that is excellent in transporting the generated electrons. Specifically, the above-described substances for forming the electron-transport layer 114 (e.g., a metal complex or a heteroaromatic compound) can be used, for example. As the electron donor, a substance having an electron-donating property with respect to the organic compound is used. Specifically, it is preferable to use an alkali metal, an alkaline earth metal, or a rare earth metal, such as lithium, sodium, cesium, magnesium, calcium, erbium, or ytterbium. It is also preferable to use an alkali metal oxide or an alkaline earth metal oxide, such as lithium oxide, calcium oxide, or barium oxide. Alternatively, a Lewis base such as magnesium oxide can be used. Further alternatively, an organic compound such as tetrathiafulvalene (abbreviation: TTF) can be used.

Note that the light-emitting layer, the hole-injection layer, the hole-transport layer, the electron-transport layer, and the electron-injection layer described above can each be formed by an evaporation method (including a vacuum evaporation method), an ink-jet method, a coating method, a gravure printing method, or the like. Besides the above-mentioned materials, an inorganic compound such as a quantum dot or a high molecular compound (e.g., an oligomer, a dendrimer, or a polymer) may be used in the light-emitting layer, the hole-injection layer, the hole-transport layer, the electron-transport layer, and the electron-injection layer.

The quantum dot may be a colloidal quantum dot, an alloyed quantum dot, a core-shell quantum dot, or a core quantum dot, for example. The quantum dot containing elements belonging to Groups 2 and 16, elements belonging to Groups 13 and 15, elements belonging to Groups 13 and 17, elements belonging to Groups 11 and 17, or elements belonging to Groups 14 and 15 may be used. Alternatively, the quantum dot containing an element such as cadmium (Cd), selenium (Se), zinc (Zn), sulfur (S), phosphorus (P), indium (In), tellurium (Te), lead (Pb), gallium (Ga), arsenic (As), or aluminum (Al) may be used.

### <<Pair of Electrodes>>

The first electrode 101 and the second electrode 102 function as an anode and a cathode of the light-emitting device. The first electrode 101 and the second electrode 102 can be formed using a metal, an alloy, or a conductive compound, a mixture or a stack thereof, or the like.

One of the first electrode 101 and the second electrode 102 is preferably formed using a conductive material having a function of reflecting light. Examples of the conductive material include aluminum (Al) and an alloy containing Al. Examples of the alloy containing Al include an alloy containing Al and L (L represents one or more of titanium (Ti), neodymium (Nd), nickel (Ni), and lanthanum (La)), such as an alloy containing Al and Ti and an alloy containing Al, Ni, and La. Aluminum has low resistance and high light reflectivity. Aluminum is included in earth's crust in large amount and is inexpensive; therefore, it is possible to reduce costs for manufacturing a light-emitting device with aluminum. Alternatively, silver (Ag), an alloy of Ag and N (N represents one or more of yttrium (Y), Nd, magnesium (Mg), ytterbium (Yb), Al, Ti, gallium (Ga), zinc (Zn), indium (In), tungsten (W), manganese (Mn), tin (Sn), iron (Fe), Ni, copper (Cu), palladium (Pd), iridium (Ir), and gold (Au)), or the like may be used. Examples of the alloy containing silver include an alloy containing silver, palladium, and copper, an alloy containing silver and copper, an alloy containing silver and magnesium, an alloy containing silver and nickel, an alloy containing silver and gold, an alloy containing silver and ytterbium, and the like. Besides, a transition metal such as tungsten, chromium (Cr), molybdenum (Mo), copper, or titanium can be used.

Light emitted from the light-emitting layer is extracted through the first electrode 101 and/or the second electrode 102. Thus, at least one of the first electrode 101 and the second electrode 102 is preferably formed using a conductive material having a function of transmitting light. As the conductive material, a conductive material having a visible light transmittance higher than or equal to 40% and lower than or equal to 100%, preferably higher than or equal to 60% and lower than or equal to 100%, and a resistivity lower than or equal to 1×10⁻² Ω·cm can be used.

The first electrode 101 and the second electrode 102 may each be formed using a conductive material having functions of transmitting light and reflecting light. As the conductive material, a conductive material having a visible light reflectivity higher than or equal to 20% and lower than or equal to 80%, preferably higher than or equal to 40% and lower than or equal to 70%, and a resistivity lower than or equal to 1×10⁻² Ω·cm can be used. For example, one or more kinds of conductive metals and alloys, conductive compounds, and the like can be used. Specifically, a metal oxide such as indium tin oxide (hereinafter, referred to as ITO), indium tin oxide containing silicon or silicon oxide (ITSO), indium oxide-zinc oxide (indium zinc oxide), indium oxide-tin oxide containing titanium, indium titanium oxide, or indium oxide containing tungsten oxide and zinc oxide can be used. A metal thin film having a thickness that allows transmission of light (preferably, a thickness greater than or equal to 1 nm and less than or equal to 30 nm) can also be used. As the metal, Ag can be used or an alloy of Ag and Al, an alloy of Ag and Mg, an alloy of Ag and Au, an alloy of Ag and Yb, or the like can be used.

In this specification and the like, as the material transmitting light, a material that transmits visible light and has conductivity is used. Examples of the material include, in addition to the above-described oxide conductor typified by ITO, an oxide semiconductor and an organic conductor containing an organic substance. Examples of the organic conductor containing an organic substance include a composite material in which an organic compound and an electron donor (donor) are mixed and a composite material in which an organic compound and an electron acceptor (acceptor) are mixed. Alternatively, an inorganic carbon-based material such as graphene may be used. The resistivity of the material is preferably lower than or equal to 1×10⁵ Ω·cm, further preferably lower than or equal to 1×10⁴ Ω·cm.

Alternatively, the first electrode 101 and/or the second electrode 102 may be formed by stacking two or more of these materials.

In order to improve the light extraction efficiency, a material whose refractive index is higher than that of an electrode having a function of transmitting light may be formed in contact with the electrode. The material may be electrically conductive or non-conductive as long as it has a function of transmitting visible light. In addition to the oxide conductors described above, an oxide semiconductor and an organic substance are given as the examples of the material. Examples of the organic substance include the materials for the light-emitting layer, the hole-injection layer, the hole-transport layer, the electron-transport layer, and the electron-injection layer. Alternatively, an inorganic carbon-based material or a metal film thin enough to transmit light can be used. Further alternatively, a plurality of layers each having a thickness of several nanometers to several tens of nanometers may be stacked.

In the case where the first electrode 101 or the second electrode 102 functions as the cathode, the electrode preferably contains a material having a low work function (lower than or equal to 3.8 eV). For example, it is possible to use an element belonging to Group 1 or 2 of the periodic table (e.g., an alkali metal such as lithium, sodium, or cesium, an alkaline earth metal such as calcium or strontium, or magnesium), an alloy containing any of these elements (e.g., Ag—Mg or Al—Li), a rare earth metal such as europium (Eu) or Yb, an alloy containing any of these rare earth metals, an alloy containing aluminum or silver, or the like.

When the first electrode 101 or the second electrode 102 is used as an anode, a material with a high work function (4.0 eV or higher) is preferably used.

The first electrode 101 and the second electrode 102 may be a stacked layer of a conductive material having a function of reflecting light and a conductive material having a function of transmitting light. This structure is preferably employed, in which case the first electrode 101 and the second electrode 102 can have a function of adjusting the optical path length so that light of a desired wavelength emitted from each light-emitting layer resonates and is intensified.

As the method for forming the first electrode 101 and the second electrode 102, a sputtering method, an evaporation method, a printing method, a coating method, a molecular beam epitaxy (MBE) method, a CVD method, a pulsed laser deposition method, an atomic layer deposition (ALD) method, or the like can be used as appropriate.

### <<Charge-Generation Layer>>

The charge-generation layer 106 has a function of injecting electrons into the organic compound layer 103a and injecting holes into the organic compound layer 103b when voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these layers may be stacked. Furthermore, an electron-relay layer may be provided between the p-type layer and the electron-injection buffer layer. Note that forming the charge-generation layer 106 with the use of any of the above materials can suppress an increase in driving voltage caused by the stack of the EL layers.

In the case where the charge-generation layer 106 is a p-type layer in which an electron acceptor is added to a hole-transport material, which is an organic compound, any of the materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films containing the respective materials may be used.

In the case where the charge-generation layer 106 is an electron-injection buffer layer in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An organic compound such as tetrathianaphthacene may be used as the electron donor.

When an electron-relay layer is provided between a p-type layer and an electron-injection buffer layer in the charge-generation layer 106, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer is preferably higher than or equal to −5.0 eV, further preferably higher than or equal to −5.0 eV and lower than or equal to −3.0 eV. Note that as the substance having an electron-transport property in the electron-relay layer, a phthalocyanine-based material or a metal complex having a metal-oxygen bond and an aromatic ligand is preferably used.

Although FIG. 3D illustrates the structure in which two of the organic compound layers 103 are stacked, three or more EL layers may be stacked with charge-generation layers each provided between two adjacent EL layers.

### <<Cap Layer>>

Although not illustrated in FIGS. 3A to 3E, a cap layer may be provided over the second electrode 102 of the light-emitting device. For example, a material with a high refractive index can be used for the cap layer. When the cap layer is provided over the second electrode 102, extraction efficiency of light emitted from the second electrode 102 can be improved.

Specific examples of a material that can be used for the cap layer include 5,5′-diphenyl-2,2′-di-5H-[1]benzothieno[3,2-c]carbazole (abbreviation: BisBTc) and 4,4′,4″-(benzene-1,3,5-triyl)tri(dibenzothiophene) (abbreviation: DBT3P-II). In addition, the organic compound described in Embodiment 1 can be used.

### <<Substrate>>

A light-emitting device of one embodiment of the present invention may be formed over a substrate of glass, plastic, or the like. As the way of stacking layers over the substrate, layers may be sequentially stacked from the first electrode 101 side or sequentially stacked from the second electrode 102 side.

For the substrate over which the light-emitting device of one embodiment of the present invention can be formed, glass, quartz, plastic, or the like can be used, for example. Alternatively, a flexible substrate may be used. The flexible substrate means a substrate that can be bent, such as a plastic substrate made of polycarbonate or polyarylate, for example. Alternatively, a film, an inorganic vapor deposition film, or the like can be used. Another material may be used as long as the substrate functions as a support in a manufacturing process of the light-emitting device or an optical device or as long as it has a function of protecting the light-emitting device or an optical device.

In this specification and the like, a light-emitting device can be formed using any of a variety of substrates, for example. There is no particular limitation on the type of the substrate. Examples of the substrate include a semiconductor substrate (e.g., a single crystal substrate such as a silicon substrate); an SOI substrate; a glass substrate; a quartz substrate; a plastic substrate; a metal substrate; a stainless steel substrate; a substrate including stainless steel foil; a tungsten substrate; a substrate including tungsten foil; a flexible substrate; an attachment film; and cellulose nanofiber (CNF), paper, and a base material film that include a fibrous material. As examples of a glass substrate, a barium borosilicate glass substrate, an aluminoborosilicate glass substrate, a soda lime glass substrate, and the like can be given. Examples of the flexible substrate, the attachment film, the base material film, and the like are substrates of plastics typified by polyethylene terephthalate (PET), polyethylene naphthalate (PEN), polyether sulfone (PES), and polytetrafluoroethylene (PTFE). Another example is an acrylic resin. Furthermore, polypropylene, polyester, polyvinyl fluoride, and polyvinyl chloride can be given as examples. Other examples include a resin such as a polyamide resin, a polyimide resin, an aramid resin, or an epoxy resin, an inorganic vapor deposition film, and paper.

Alternatively, a flexible substrate may be used as the substrate, and a light-emitting device may be provided directly on the flexible substrate. Further alternatively, a separation layer may be provided between the substrate and the light-emitting device. The separation layer can be used to separate part or the whole of the light-emitting device, which is formed over the separation layer, from the substrate and transfer the separated component onto another substrate. In that case, the light-emitting device can be transferred to a substrate having low heat resistance or a flexible substrate as well. For the above separation layer, a stack including inorganic films, which are a tungsten film and a silicon oxide film, or a structure in which a resin film of polyimide or the like is formed over a substrate can be used, for example.

In other words, after the light-emitting device is formed using a substrate, the light-emitting device may be transferred to another substrate. Examples of the substrate to which the light-emitting device is transferred are, in addition to the above substrates, a cellophane substrate, a stone substrate, a wood substrate, a cloth substrate (including a natural fiber (e.g., silk, cotton, or hemp), a synthetic fiber (e.g., nylon, polyurethane, or polyester), a regenerated fiber (e.g., acetate, cupro, rayon, or regenerated polyester), and the like), a leather substrate, a rubber substrate, and the like. When such a substrate is used, a light-emitting device with high durability, high heat resistance, reduced weight, or reduced thickness can be formed.

The light-emitting device may be formed over an electrode electrically connected to a field-effect transistor (FET) that is formed over any of the above-described substrates, for example. Accordingly, an active matrix display apparatus in which the FET controls the driving of the light-emitting device can be manufactured.

In this embodiment, one embodiment of the present invention has been described. Other embodiments of the present invention are described in other embodiments. Note that one embodiment of the present invention is not limited thereto. In other words, various embodiments of the invention are described in this embodiment and the other embodiments, and one embodiment of the present invention is not limited to a particular embodiment. For example, although the example in which one embodiment of the present invention is applied to a light-emitting device is described, one embodiment of the present invention is not limited thereto. For example, depending on circumstances or conditions, one embodiment of the present invention is not necessarily used in a light-emitting device. One embodiment of the present invention shows, but is not limited to, an example of including a first organic compound, a second organic compound, and a guest material capable of converting triplet excitation energy into light emission, in which the LUMO level of the first organic compound is lower than that of the second organic compound and the HOMO level of the first organic compound is lower than that of the second organic compound. Depending on circumstances or conditions, in one embodiment of the present invention, for example, the LUMO level of the first organic compound is not necessarily lower than that of the second organic compound. Alternatively, the HOMO level of the first organic compound is not necessarily lower than that of the second organic compound. One embodiment of the present invention shows, but is not limited to, an example in which the first organic compound and the second organic compound form an exciplex. Depending on circumstances or conditions, in one embodiment of the present invention, for example, the first organic compound and the second organic compound do not necessarily form an exciplex. One embodiment of the present invention shows, but is not limited to, an example in which the LUMO level of the guest material is higher than that of the first organic compound and the HOMO level of the guest material is lower than that of the second organic compound. Depending on circumstances or conditions, in one embodiment of the present invention, for example, the LUMO level of the guest material is not necessarily higher than that of the first organic compound. Alternatively, the HOMO level of the guest material is not necessarily lower than that of the second organic compound.

The structure described above in this embodiment can be combined as appropriate with any of the structures described in the other embodiments.

### Embodiment 3

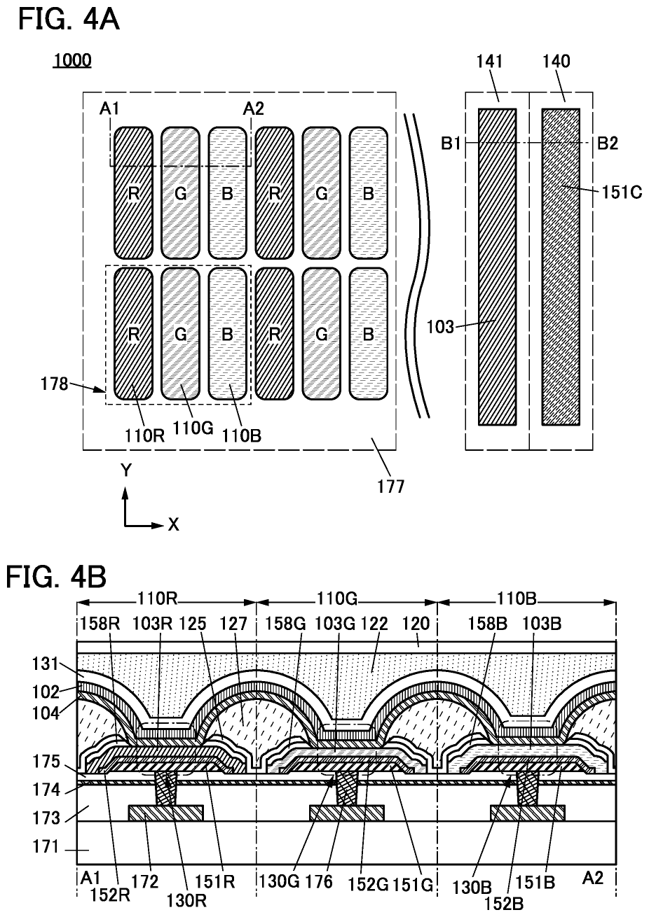

As illustrated as an example in FIGS. 4A and 4B, a plurality of the light-emitting devices described in the above embodiment are formed over an insulating layer 175 to constitute a light-emitting apparatus. In this embodiment, the light-emitting apparatus of one embodiment of the present invention will be described in detail.

A light-emitting apparatus 1000 includes a pixel portion 177 in which a plurality of pixels 178 are arranged in matrix. The pixel 178 includes a subpixel 110R, a subpixel 110G, and a subpixel 110n.

In this specification and the like, for example, matters common to the subpixels 110R, 110G, and 110B are sometimes described using the collective term “subpixel 110”. As for components that are distinguished from each other using letters of the alphabet, matters common to the components are sometimes described using reference numerals not accompanied with the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by subpixels; however, the structure of the present invention is not limited to this structure. That is, subpixels of a different combination of colors may be employed. The number of subpixels is not limited to three, and four or more subpixels may be used, for example. Examples of four subpixels include subpixels emitting light of four colors of R, G, B, and white (W), subpixels emitting light of four colors of R, G, B, and yellow (Y), and four subpixels emitting light of R, G, and B and infrared light (IR).

In this specification and the like, the row direction and the column direction are sometimes referred to as the X direction and the Y direction, respectively. The X direction and the Y direction intersect with each other and are perpendicular to each other, for example.

FIG. 4A illustrates an example where subpixels of different colors are arranged in the X direction and subpixels of the same color are arranged in the Y direction. Note that subpixels of different colors may be arranged in the Y direction, and subpixels of the same color may be arranged in the X direction.

A connection portion 140 and a region 141 may be provided outside the pixel portion 177. The region 141 is preferably positioned between the pixel portion 177 and the connection portion 140, for example. The organic compound layer 103 is provided in the region 141. A conductive layer 151C is provided in the connection portion 140.

Although FIG. 4A illustrates an example where the region 141 and the connection portion 140 are positioned on the right side of the pixel portion 177, there is no particular limitation on the positions of the region 141 and the connection portion 140. The number of regions 141 and the number of connection portions 140 can each be one or two or more.

FIG. 4B is an example of a cross-sectional view along the dashed-dotted line A1-A2 in FIG. 4A. As illustrated in FIG. 4B, the light-emitting apparatus 1000 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is preferably provided over a substrate (not illustrated). An opening reaching the conductive layer 172 is provided in the insulating layers 175, 174, and 173, and a plug 176 is provided to fill the opening.

In the pixel portion 177, a light-emitting device 130 is provided over the insulating layer 175 and the plug 176. A protective layer 131 is provided to cover the light-emitting device 130. A substrate 120 is bonded to the protective layer 131 with a resin layer 122. An inorganic insulating layer 125 and an insulating layer 127 over the inorganic insulating layer 125 may be provided between adjacent light-emitting devices 130.

Although FIG. 4B illustrates cross sections of a plurality of the inorganic insulating layers 125 and a plurality of the insulating layers 127, the inorganic insulating layers 125 are preferably connected to each other and the insulating layers 127 are preferably connected to each other when the light-emitting apparatus 1000 is seen from above. That is, the inorganic insulating layer 125 and the insulating layer 127 preferably have openings above first electrodes.

In FIG. 4B, a light-emitting device 130R, a light-emitting device 130G, and a light-emitting device 130B are each illustrated as the light-emitting device 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, the light-emitting device 130G, or the light-emitting device 130B may emit visible light of another color or infrared light.

Note that the organic compound layer 103 at least includes a light-emitting layer and can include other functional layers (a hole-injection layer, a hole-transport layer, a hole-blocking layer, an electron-blocking layer, an electron-transport layer, an electron-injection layer, and the like). A combination of the organic compound layer 103 and a common layer 104 may constitute functional layers (a hole-injection layer, a hole-transport layer, a hole-blocking layer, a light-emitting layer, an electron-blocking layer, an electron-transport layer, an electron-injection layer, and the like) included in the EL layer that emits light.

The light-emitting apparatus of one embodiment of the present invention can be, for example, a top-emission light-emitting apparatus where light is emitted in the direction opposite to a substrate over which light-emitting devices are formed. Note that the light-emitting apparatus of one embodiment of the present invention may be of a bottom-emission type.

The light-emitting device 130R has a structure as described in the above embodiments. The light-emitting device 130R includes the first electrode (pixel electrode) including a conductive layer 151R and a conductive layer 152R, an organic compound layer 103R over the first electrode, the common layer 104 over the organic compound layer 103R, and the second electrode (common electrode) 102 over the common layer 104.

Note that the common layer 104 is not necessarily provided. The common layer 104 can reduce damage to the organic compound layer 103R caused in a later step. In the case where the common layer 104 is provided, the common layer 104 may function as an electron-injection layer. In the case where the common layer 104 functions as an electron-injection layer, a stack of the organic compound layer 103R and the common layer 104 corresponds to the organic compound layer 103 in Embodiment 1.

Each of the light-emitting devices 130 has a structure as described in Embodiment 2 and includes the first electrode (pixel electrode) including a conductive layer 151 and a conductive layer 152, the organic compound layer 103 over the first electrode, the common layer 104 over the organic compound layer 103, and the second electrode (common electrode) 102 over the common layer 104.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layer 103R, the organic compound layer 103G, and the organic compound layer 103B are island-shaped layers and are isolated on a light-emitting device basis or on an emission color basis. Providing the island-shaped organic compound layer 103 in each of the light-emitting devices 130 can suppress leakage current between the adjacent light-emitting devices 130 even in a high-definition light-emitting apparatus. This can prevent crosstalk, so that a light-emitting apparatus with extremely high contrast can be provided. Specifically, a light-emitting apparatus having high current efficiency at low luminance can be provided.

The organic compound layer 103 may be provided to cover the top and side surfaces of the first electrode (pixel electrode) of the light-emitting device 130. In that case, the aperture ratio of the light-emitting apparatus 1000 can be easily increased as compared to the structure where an edge portion of the organic compound layer 103 is positioned inward from an edge portion of the pixel electrode. Covering the side surface of the pixel electrode of the light-emitting device 130 with the organic compound layer 103 can inhibit the pixel electrode from being in contact with the second electrode 102; hence, a short circuit of the light-emitting device 130 can be inhibited. Furthermore, the distance between a light-emitting region (i.e., a region overlapping with the pixel electrode) in the organic compound layer 103 and the edge portion of the organic compound layer 103 can be increased. Since the edge portion of the organic compound layer 103 might be damaged by processing, using a region that is away from the edge portion of the organic compound layer 103 as the light-emitting region can increase the reliability of the light-emitting device 130.

In the light-emitting apparatus of one embodiment of the present invention, the first electrode (pixel electrode) of the light-emitting device may have a stacked-layer structure. For example, in the example illustrated in FIG. 4B, the first electrode of the light-emitting device 130 is a stack-layer structure of the conductive layer 151 (conductive layers 151R, 151G, 151B) and the conductive layer 152 (conductive layers 152R, 152G, 152B).

In the case where the light-emitting apparatus 1000 is a top-emission light-emitting apparatus, for example, in the pixel electrode of the light-emitting device 130, the conductive layer 151 preferably has high visible light reflectance and the conductive layer 152 preferably has a visible-light-transmitting property and a high work function. The higher the visible light reflectance of the pixel electrode is, the higher the efficiency of extraction of the light emitted by the organic compound layer 103 is. In the case where the pixel electrode functions as an anode, the higher the work function of the pixel electrode is, the easier it is to inject holes into the organic compound layer 103. Accordingly, when the pixel electrode of the light-emitting device 130 has a stacked-layer structure of the conductive layer 151 with high visible light reflectance and the conductive layer 152 with a high work function, the light-emitting device 130 can have high light extraction efficiency and a low driving voltage.

Specifically, the visible light reflectance of the conductive layer 151 is preferably higher than or equal to 40% and lower than or equal to 100%, further preferably higher than or equal to 70% and lower than or equal to 100%, for example. When the conductive layer 152 is used as an electrode having a visible-light-transmitting property, the visible light transmittance is preferably higher than or equal to 40%, for example.

In the case where a film formed after the formation of the pixel electrode having a stacked-layer structure is removed by a wet etching method, for example, the structure might be impregnated with a chemical solution used for the etching. When the chemical solution reaches the pixel electrode, galvanic corrosion between a plurality of layers constituting the pixel electrode might occur, leading to deterioration of the pixel electrode.

In view of the above, the conductive layer 152 is preferably formed to cover the top and side surfaces of the conductive layer 151. When the conductive layer 151 is covered with the conductive layer 152, the chemical solution does not reach the conductive layer 151; thus, occurrence of galvanic corrosion in the pixel electrode can be suppressed. This allows the light-emitting apparatus 1000 to be manufactured in a high yield and at low cost. In addition, generation of a defect in the light-emitting apparatus 1000 can be suppressed, which makes the light-emitting apparatus 1000 highly reliable.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, indium tin oxide containing silicon can be suitably used for the conductive layer 152 because of having a high work function, for example, a work function higher than or equal to 4.0 eV.

The conductive layer 151 and the conductive layer 152 may have a stacked-layer structure of a plurality of layers containing different materials. In that case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 has a stacked-layer structure of two or more layers, for example, a layer in contact with the conductive layer 152 can contain the same material as a layer of the conductive layer 152 that is in contact with the conductive layer 151.

The conductive layer 151 preferably has an edge portion with a tapered shape. Specifically, the edge portion of the conductive layer 151 preferably has a tapered shape with a taper angle of less than 90°. In that case, the conductive layer 152 provided along the side surface of the conductive layer 151 also has a tapered shape. When an edge portion of the conductive layer 152 has a tapered shape, coverage with the organic compound layer 103 provided along the side surface of the conductive layer 152 can be improved.

In the case where the conductive layer 151 or the conductive layer 152 has a stacked-layer structure, at least one of the stacked layers preferably has a tapered side surface. The stacked layers of the conductive layer(s) may have different tapered shapes from each other.

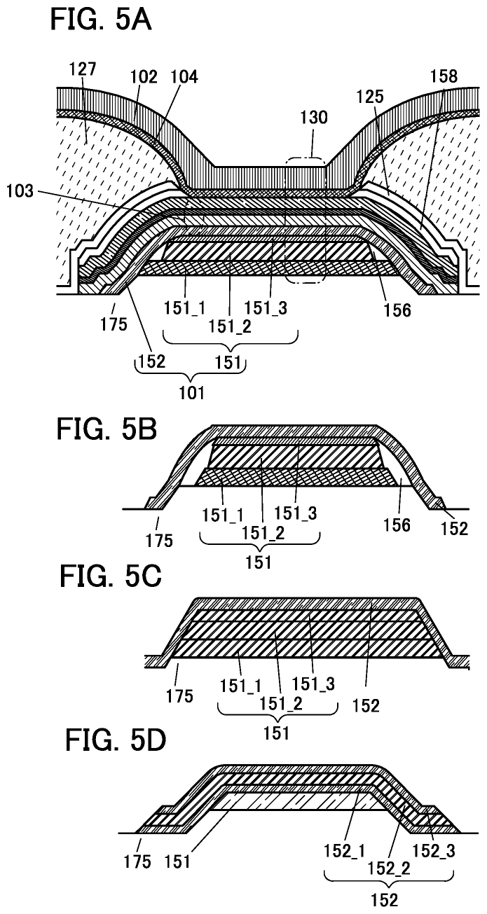

FIG. 5A illustrates the case where the conductive layer 151 has a stacked-layer structure of a plurality of layers containing different materials. As illustrated in FIG. 5A, the conductive layer 151 includes a conductive layer 151_1, a conductive layer 151_2 over the conductive layer 151_1, and a conductive layer 151_3 over the conductive layer 151_2. In other words, the conductive layer 151 illustrated in FIG. 5A has a three-layer structure. In the case where the conductive layer 151 is a stacked-layer structure of a plurality of layers as described above, the visible light reflectance of at least one of the layers included in the conductive layer 151 is made higher than that of the conductive layer 152.

In the example illustrated in FIG. 5A, the conductive layer 1512 is interposed between the conductive layers 151_1 and 151_3. A material that is less likely to change in quality than the material for the conductive layer 151_2 is preferably used for the conductive layers 151_1 and 151_3. The conductive layer 151_1 can be formed using, for example, a material that is less likely to migrate owing to contact with the insulating layer 175 than the material for the conductive layer 151_2. The conductive layer 151_3 can be formed using a material an oxide of which has lower electrical resistivity than an oxide of the material used for the conductive layer 151_2 and which is less likely to be oxidized than the conductive layer 151_2.

In this manner, the structure in which the conductive layer 1512 is interposed between the conductive layers 151_1 and 151_3 can expand the range of choices for the material for the conductive layer 151_2. The conductive layer 1512, for example, can thus have higher visible light reflectance than at least one of the conductive layers 151_1 and 151_3. For example, aluminum can be used for the conductive layer 151_2. The conductive layer 151_2 may be formed using an alloy containing aluminum. The conductive layer 1511 can be formed using titanium; titanium has lower visible light reflectance than aluminum but is less likely to migrate by contact with the insulating layer 175 than aluminum. Furthermore, the conductive layer 151_3 can be formed using titanium; titanium is less likely to be oxidized than aluminum and an oxide of titanium has lower electrical resistivity than aluminum oxide, although titanium has lower visible light reflectance than aluminum.

The conductive layer 151_3 may be formed using silver or an alloy containing silver. Silver has a visible light reflectance higher than that of titanium. In addition, silver is less likely to be oxidized than aluminum, and silver oxide has electrical resistivity lower than that of aluminum oxide. Thus, the conductive layer 151_3 formed using silver or an alloy containing silver can favorably increase the visible light reflectance of the conductive layer 151 and suppress an increase in the electric resistance of the pixel electrode due to oxidation of the conductive layer 151_2. Here, as the alloy containing silver, an alloy of silver, palladium, and copper (Ag—Pd—Cu, also referred to as APC) can be used, for example. When the conductive layer 151_3 is formed using silver or an alloy containing silver and the conductive layer 151_2 is formed using aluminum, the visible light reflectance of the conductive layer 151_3 can be higher than that of the conductive layer 151_2. Here, the conductive layer 151_2 may be formed using silver or an alloy containing silver. The conductive layer 151_1 may be formed using silver or an alloy containing silver.

Meanwhile, a film formed using titanium has better processability in etching than a film formed using silver. Thus, the use of titanium for the conductive layer 151_3 can facilitate formation of the conductive layer 151_3. Note that a film formed using aluminum also has better processability in etching than a film formed using silver.

The conductive layer 151 having a stacked-layer structure of a plurality of layers as described above can improve the characteristics of the light-emitting apparatus. For example, the light-emitting apparatus 1000 can have high light extraction efficiency and high reliability.

Here, in the case where the light-emitting device 130 has a microcavity structure, use of silver or an alloy containing silver, i.e., a material with high visible light reflectance, for the conductive layer 151_3 can favorably increase the light extraction efficiency of the light-emitting apparatus 1000.

Depending on the selected material or the processing method of the conductive layer 151, the side surface of the conductive layer 151_2 is positioned inward from the side surfaces of the conductive layer 151_1 and the conductive layer 151_3 and a protruding portion might be formed as illustrated in FIG. 5A. This might impair coverage of the conductive layer 151 with the conductive layer 152 to cause step disconnection of the conductive layer 152.

Thus, an insulating layer 156 is preferably provided as illustrated in FIG. 5A. FIG. 5A illustrates an example in which the insulating layer 156 is provided over the conductive layer 1511 to include a region overlapping with the side surface of the conductive layer 151_2. Such a structure can inhibit occurrence of the step disconnection or a reduction in the thickness of the conductive layer 152 due to the protruding portion; thus, connection defects or an increase in driving voltage can be inhibited.

Although FIG. 5A illustrates the structure in which the side surface of the conductive layer 1512 is entirely covered with the insulating layer 156, part of the side surface the conductive layer 151_2 is not necessarily covered with the insulating layer 156. Also in a pixel electrode with a later-described structure, part of the side surface of the conductive layer 151_2 is not necessarily covered with the insulating layer 156.

The insulating layer 156 preferably has a curved surface as illustrated in FIG. 5A. In that case, step disconnection in the conductive layer 152 covering the insulating layer 156 is less likely to occur than in the case where the insulating layer 156 has a perpendicular side surface, for example. In addition, step disconnection in the conductive layer 152 covering the insulating layer 156 is less likely to occur also in the case where the side surface of the insulating layer 156 has a tapered shape, or specifically, a tapered shape with a taper angle of less than 90°, than in the case where the insulating layer 156 has a perpendicular side surface, for example. As described above, the light-emitting apparatus 1000 can be fabricated in a high yield. Moreover, the light-emitting apparatus 1000 can have high reliability since generation of defects is suppressed therein.

Note that one embodiment of the present invention is not limited thereto. FIGS. 5B to 5D illustrate other examples of the structure of the first electrode 101.

FIG. 5B illustrates a variation structure of the first electrode 101 in FIG. 5A, in which the insulating layer 156 covers the side surfaces of the conductive layers 151_1, 151_2, and 151_3 instead of covering only the side surface of the conductive layer 151_2.

FIG. 5C illustrates a variation structure of the first electrode 101 in FIG. 5A, in which the insulating layer 156 is not provided.

FIG. 5D illustrates a variation structure of the first electrode 101 in FIG. 5A, in which the conductive layer 151 does not have a stacked-layer structure but the conductive layer 152 has a stacked-layer structure.

A conductive layer 152_1 has higher adhesion to a conductive layer 152_2 than the insulating layer 175 does, for example. For the conductive layer 152_1, an oxide containing one or more of indium, tin, zinc, gallium, titanium, aluminum, and silicon, for example, can be used. For example, it is preferable to use a conductive oxide containing one or more of indium oxide, indium tin oxide, indium zinc oxide, zinc oxide, zinc oxide containing gallium, titanium oxide, indium titanium oxide, zinc titanium oxide, aluminum zinc oxide, indium zinc oxide containing gallium, indium zinc oxide containing aluminum, indium tin oxide containing silicon, indium zinc oxide containing silicon, and the like. Accordingly, peeling of the conductive layer 1522 can be suppressed. The conductive layer 1522 is not in contact with the insulating layer 175.

The conductive layer 152_2 is a layer whose visible light reflectance (e.g., reflectance with respect to light with a predetermined wavelength longer than or equal to 400 nm and shorter than 750 nm) is higher than that of the conductive layers 151, 152_1, and 152_3. The visible light reflectance of the conductive layer 152_2 can be, for example, higher than or equal to 70% and lower than or equal to 100%, and is preferably higher than or equal to 80% and lower than or equal to 100%, further preferably higher than or equal to 90% and lower than or equal to 100%. For the conductive layer 152_2, silver or an alloy containing silver can be used, for example. An example of the alloy containing silver is an alloy of silver, palladium, and copper (APC). In the above manner, the light-emitting apparatus 1000 can have high light extraction efficiency. Note that a metal other than silver may be used for the conductive layer 152_2.

When the conductive layers 151 and 152 serve as the anode, a layer having a high work function is preferably used as the conductive layer 152_3. The conductive layer 1523 has a higher work function than the conductive layer 1522, for example. For the conductive layer 1523, a material similar to the material usable for the conductive layer 1521 can be used, for example. The conductive layers 1521 and 152_3 can be formed using the same kind of material, for example.

When the conductive layers 151 and 152 serve as the cathode, a layer having a low work function is preferably used as the conductive layer 152_3. The conductive layer 1523 has a lower work function than the conductive layer 1522, for example.

The conductive layer 152_3 is preferably a layer having high visible light transmittance (e.g., transmittance with respect to light with a predetermined wavelength longer than or equal to 400 nm and shorter than 750 nm). For example, the visible light transmittance of the conductive layer 152_3 is preferably higher than those of the conductive layers 151 and 152_2. The visible light transmittance of the conductive layer 152_3 can be, for example, higher than or equal to 60% and lower than or equal to 100%, and is preferably higher than or equal to 70% and lower than or equal to 100% further preferably higher than or equal to 80% and lower than or equal to 100%. Accordingly, the amount of light absorbed by the conductive layer 152_3 among light emitted from the organic compound layer 103 can be reduced. As described above, the conductive layer 1522 under the conductive layer 152_3 can be a layer having high visible light reflectance. Thus, the light-emitting apparatus 1000 can have high light extraction efficiency.

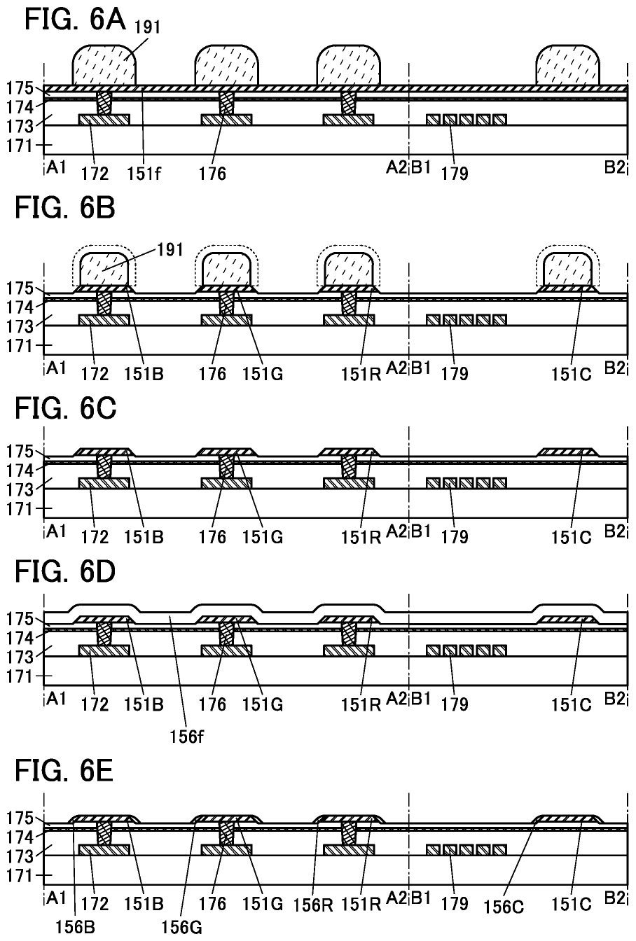

Next, an exemplary method for fabricating the light-emitting apparatus 1000 having the structure illustrated in FIGS. 4A and 4B is described with reference to FIGS. 6A to 6E, FIGS. 7A to 7E, FIGS. 8A to 8C, FIGS. 9A to 9C, FIGS. 10A to 10C, FIGS. 11A to 11C, and FIGS. 12A to 12C.

### Fabrication Method Example 1

Thin films included in the light-emitting apparatus (e.g., insulating films, semiconductor films, and conductive films) can be formed by a sputtering method, a chemical vapor deposition (CVD) method, a vacuum evaporation method, a pulsed laser deposition (PLD) method, an ALD method, or the like. Examples of a CVD method include a plasma-enhanced CVD (PECVD) method and a thermal CVD method. An example of a thermal CVD method is a metal organic CVD (MOCVD) method.

Thin films included in the light-emitting apparatus (e.g., insulating films, semiconductor films, and conductive films) can also be formed by a wet process such as spin coating, dipping, spray coating, ink-jetting, dispensing, screen printing, offset printing, doctor blade coating, slit coating, roll coating, curtain coating, or knife coating.

Specifically, for fabrication of the light-emitting device, a vacuum process such as an evaporation method and a solution process such as a spin coating method or an ink-jet method can be used. Examples of an evaporation method include physical vapor deposition methods (PVD methods) such as a sputtering method, an ion plating method, an ion beam evaporation method, a molecular beam evaporation method, and a vacuum evaporation method, and a chemical vapor deposition method (CVD method). Specifically, the functional layers (e.g., the hole-injection layer, the hole-transport layer, the hole-blocking layer, the light-emitting layer, the electron-blocking layer, the electron-transport layer, and the electron-injection layer) included in the organic compound layer can be formed by an evaporation method (e.g., a vacuum evaporation method), a coating method (e.g., a dip coating method, a die coating method, a bar coating method, a spin coating method, or a spray coating method), a printing method (e.g., ink-jetting, screen printing (stencil), offset printing (planography), flexography (relief printing), gravure printing, or micro-contact printing), or the like.

Thin films included in the light-emitting apparatus can be processed by a photolithography method, for example. Alternatively, a nanoimprinting method, a sandblasting method, a lift-off method, or the like may be used to process thin films. Alternatively, island-shaped thin films may be directly formed by a film formation method using a shielding mask such as a metal mask.

There are two typical examples of photolithography methods. In one of the methods, a resist mask is formed over a thin film that is to be processed, the thin film is processed by etching, for example, and then the resist mask is removed. In the other method, a photosensitive thin film is formed and then processed into a desired shape by light exposure and development.

For etching of thin films, a dry etching method, a wet etching method, a sandblast method, or the like can be used.

First, as illustrated in FIG. 6A, the insulating layer 171 is formed over a substrate (not illustrated). Next, the conductive layer 172 and a conductive layer 179 are formed over the insulating layer 171, and the insulating layer 173 is formed over the insulating layer 171 so as to cover the conductive layer 172 and the conductive layer 179. Then, the insulating layer 174 is formed over the insulating layer 173, and the insulating layer 175 is formed over the insulating layer 174.

As the substrate, a substrate that has heat resistance high enough to withstand at least heat treatment performed later can be used. When an insulating substrate is used as the substrate, it is possible to use a glass substrate, a quartz substrate, a sapphire substrate, a ceramic substrate, an organic resin substrate, or the like. Alternatively, it is possible to use a semiconductor substrate such as a single crystal semiconductor substrate or a polycrystalline semiconductor substrate of silicon, silicon carbide, or the like, a compound semiconductor substrate of silicon germanium or the like, or an SOI substrate.

Next, as illustrated in FIG. 6A, openings reaching the conductive layer 172 are formed in the insulating layers 175, 174, and 173. Then, the plugs 176 are formed to fill the openings.

Next, as illustrated in FIG. 6A, a conductive film 151f to be the conductive layers 151R, 151G, 151B, and 151C is formed over the plugs 176 and the insulating layer 175. The conductive film 151f can be formed by a sputtering method or a vacuum evaporation method, for example. A metal material can be used for the conductive film 151f, for example.

Subsequently, a resist mask 191 is formed over the conductive film 151f, for example, as illustrated in FIG. 6A. The resist mask 191 can be formed by application of a photosensitive material (photoresist), light exposure, and development.

Subsequently, as illustrated in FIG. 6B, the conductive film 151f in a region that does not overlap with the resist mask 191, for example, is removed by an etching method, specifically, a dry etching method, for instance. Note that in the case where the conductive film 151f includes a layer formed using a conductive oxide such as indium tin oxide, for example, the layer may be removed by a wet etching method. In this manner, the conductive layer 151 is formed. In the case where part of the conductive film 151f is removed by a dry etching method, for example, a recessed portion (also referred to as a depression) may be formed in a region of the insulating layer 175 that does not overlap with the conductive layer 151.

Next, the resist mask 191 is removed as illustrated in FIG. 6C. The resist mask 191 can be removed by ashing using oxygen plasma, for example. Alternatively, an oxygen gas and any of CF₄, C₄F₈, SF₆, CHF₃, Cl₂, H₂O, BCl₃, and a Group 18 element such as He may be used. Alternatively, the resist mask 191 may be removed by wet etching.

Then, as illustrated in FIG. 6D, an insulating film 156f to be an insulating layer 156R, an insulating layer 156G, an insulating layer 156B, and an insulating layer 156C is formed over the conductive layer 151R, the conductive layer 151G, the conductive layer 151B, the conductive layer 151C, and the insulating layer 175. The insulating film 156f can be formed by a CVD method, an ALD method, a sputtering method, or a vacuum evaporation method, for example.

For the insulating film 156f, an inorganic material can be used. As the insulating film 156f, an inorganic insulating film such as an oxide insulating film, a nitride insulating film, an oxynitride insulating film, or a nitride oxide insulating film can be used, for example. For example, an oxide insulating film containing silicon, a nitride insulating film containing silicon, an oxynitride insulating film containing silicon, a nitride oxide insulating film containing silicon, or the like can be used as the insulating film 156f. For the insulating film 156f, silicon oxynitride can be used, for example.

Subsequently, as illustrated in FIG. 6E, the insulating film 156f is processed to form the insulating layers 156R, 156G, 156B, and 156C. The insulating layer 156 can be formed by performing etching substantially uniformly on the top surface of the insulating film 156f, for example. Such uniform etching for planarization is also referred to as etch back treatment. Note that the insulating layer 156 may be formed by a photolithography method.

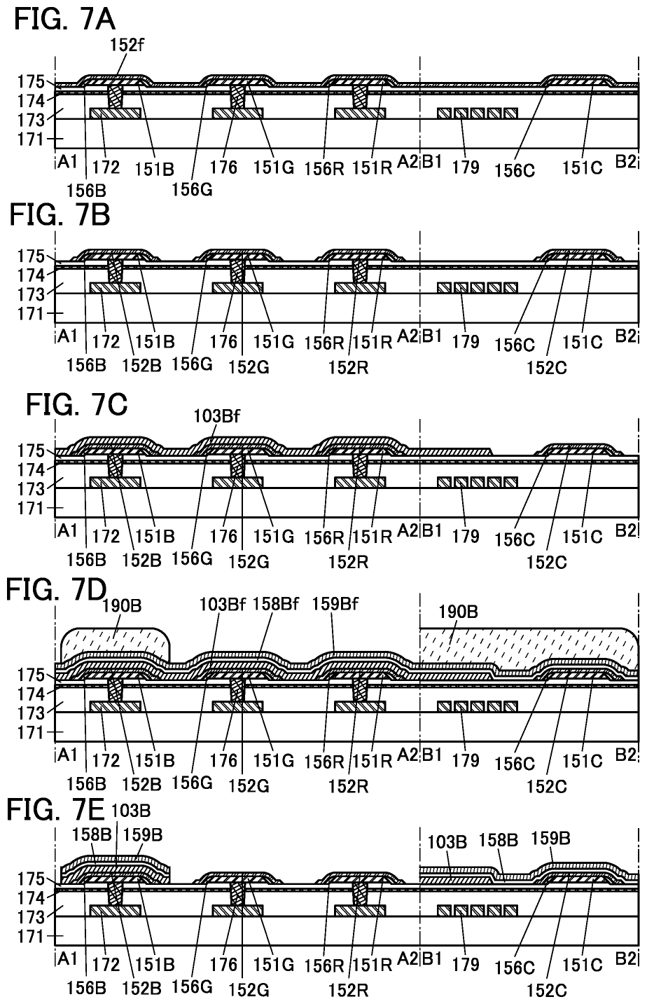

Then, as illustrated in FIG. 7A, a conductive film 152f to be conductive layers 152R, 152G, 152B, and 152C is formed over the conductive layers 151R, 151G, 151B, and 151C and the insulating layers 156R, 156G, 156B, 156C, and 175. Specifically, the conductive film 152f is formed to cover the conductive layers 151R, 151G, 151B, and 151C and the insulating layers 156R, 156G, 156B, and 156C, for example.

The conductive film 152f can be formed by a sputtering method or a vacuum evaporation method, for example. The conductive film 152f can be formed by an ALD method. A conductive oxide can be used for the conductive film 152f, for example. The conductive film 152f can have a stacked-layer structure of a film formed using a metal material and a film formed using a conductive oxide thereover. For example, the conductive film 152f can have a stacked-layer structure of a film formed using titanium, silver, or an alloy containing silver and a film formed using a conductive oxide thereover.

Then, as illustrated in FIG. 7B, the conductive film 152f is processed by a photolithography method, for example, so that the conductive layers 152R, 152G, 152B, and 152C are formed. Specifically, after a resist mask is formed, part of the conductive film 152f is removed by an etching method, for example. The conductive film 152f can be removed by a wet etching method, for example. The conductive film 152f may be removed by a dry etching method. Through the above steps, the pixel electrode including the conductive layer 151 and the conductive layer 152 is formed.

Next, hydrophobization treatment is preferably performed on the conductive layer 152. The hydrophobization treatment can change the hydrophilic properties of the subject surface to hydrophobic properties or increase the hydrophobic properties of the subject surface. The hydrophobization treatment for the conductive layer 152 can increase the adhesion between the conductive layer 152 and the organic compound layer 103 formed in a later step to reduce film peeling. Note that the hydrophobization treatment is not necessarily performed.

Next, as illustrated in FIG. 7C, an organic compound film 103Bf to be the organic compound layer 103B is formed over the conductive layers 152B, 152G, and 152R and the insulating layer 175.

Note that in the present invention, the organic compound film 103Bf includes a plurality of organic compound layers including at least one light-emitting layer. The structure of the light-emitting device 130 described in Embodiment 2 can be referred to for the specific structure. The plurality of organic compound layers including at least one light-emitting layer may be stacked with an intermediate layer positioned therebetween.

As illustrated in FIG. 7C, the organic compound film 103Bf is not formed over the conductive layer 152C. For example, a mask for defining a film formation area (also referred to as an area mask, a rough metal mask, or the like to be distinguished from a fine metal mask) is used, so that the organic compound film 103Bf can be formed only in a desired region. Employing a film formation step using an area mask and a processing step using a resist mask enables a light-emitting device to be fabricated by a relatively easy process.

The organic compound film 103Bf can be formed by an evaporation method, specifically a vacuum evaporation method. The organic compound film 103Bf may be formed by a transfer method, a printing method, an ink-jet method, a coating method, or the like.

Next, as illustrated in FIG. 7D, a sacrificial film 158Bf to be a sacrificial layer 158B and a mask film 159Bf to be a mask layer 159B are sequentially formed over the organic compound film 103Bf.

The sacrificial film 158Bf and the mask film 159Bf can be formed by a sputtering method, an ALD method (including a thermal ALD method and a PEALD method), a CVD method, or a vacuum evaporation method, for example. Alternatively, the sacrificial film 158Bf and the mask film 159Bf may be formed by the above-described wet process.

The sacrificial film 158Bf and the mask film 159Bf are formed at a temperature lower than the upper temperature limit of the organic compound film 103Bf. The typical substrate temperatures in formation of the sacrificial film 158Bf and the mask film 159Bf are each lower than or equal to 200° C., preferably lower than or equal to 150° C., further preferably lower than or equal to 120° C., still further preferably lower than or equal to 100° C., yet still further preferably lower than or equal to 80° C.

Although this embodiment shows an example where a mask film having a two-layer structure of the sacrificial film 158Bf and the mask film 159Bf is formed, a mask film may have a single-layer structure or a stacked-layer structure of three or more layers.

Providing the sacrificial film over the organic compound film 103Bf can reduce damage to the organic compound film 103Bf in the fabrication process of the light-emitting apparatus, leading to an increase in reliability of the light-emitting device.

As the sacrificial film 158Bf, a film that is highly resistant to the process conditions for the organic compound film 103Bf, specifically, a film having high etching selectivity with respect to the organic compound film 103Bf is used. For the mask film 159Bf, a film having high etching selectivity with respect to the sacrificial film 158Bf is used.

The sacrificial film 158Bf and the mask film 159Bf are preferably films that can be removed by a wet etching method. The use of a wet etching method can reduce damage to the organic compound film 103Bf in processing of the sacrificial film 158Bf and the mask film 159Bf, as compared to the case of using a dry etching method.

In the case where a wet etching method is employed, it is particularly preferable to use an acidic chemical solution. As an acidic chemical solution, a chemical solution containing one of phosphoric acid, hydrofluoric acid, nitric acid, acetic acid, oxalic acid, sulfuric acid, and the like or a mixed chemical solution (also referred to as a mixed acid) that contains two or more of these acids is preferably used.

As each of the sacrificial film 158Bf and the mask film 159Bf, one or more of a metal film, an alloy film, a metal oxide film, a semiconductor film, an organic insulating film, and an inorganic insulating film, for example, can be used.

When a film containing a material having a property of blocking ultraviolet rays is used as each of the sacrificial film 158Bf and the mask film 159Bf, the organic compound layer can be inhibited from being irradiated with ultraviolet rays in a light exposure step, for example. When the organic compound layer is inhibited from being damaged by ultraviolet rays, the reliability of the light-emitting device can be improved.

Note that the same effect is obtained when a film containing a material having a property of blocking ultraviolet rays is used for an inorganic insulating film 125f described later.

For each of the sacrificial film 158Bf and the mask film 159Bf, it is possible to use a metal material such as gold, silver, platinum, magnesium, nickel, tungsten, chromium, molybdenum, iron, cobalt, copper, palladium, titanium, aluminum, yttrium, zirconium, or tantalum or an alloy material containing any of the metal materials, for example. It is particularly preferable to use a low-melting-point material such as aluminum or silver.

The sacrificial film 158Bf and the mask film 159Bf can each be formed using a metal oxide such as In—Ga—Zn oxide, indium oxide, In—Zn oxide, In—Sn oxide, indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or indium tin oxide containing silicon.

In place of gallium described above, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used.

The sacrificial film 158Bf and the mask film 159Bf are preferably formed using a semiconductor material such as silicon or germanium, for example, for excellent compatibility with a semiconductor manufacturing process. An oxide or a nitride of the semiconductor material can be used. A non-metallic material such as carbon or a compound thereof can be used. A metal such as titanium, tantalum, tungsten, chromium, or aluminum or an alloy containing at least one of these metals can be used. Alternatively, an oxide containing the above-described metal, such as titanium oxide or chromium oxide, or a nitride such as titanium nitride, chromium nitride, or tantalum nitride can be used.

As each of the sacrificial film 158Bf and the mask film 159Bf, any of a variety of inorganic insulating films can be used. In particular, an oxide insulating film is preferable because its adhesion to the organic compound film 103Bf is higher than that of a nitride insulating film. For example, an inorganic insulating material such as aluminum oxide, hafnium oxide, or silicon oxide can be used for the sacrificial film 158Bf and the mask film 159Bf. As the sacrificial film 158Bf and the mask film 159Bf, aluminum oxide films can be formed by an ALD method, for example. An ALD method is preferably used, in which case damage to a base (in particular, the organic compound layer) can be reduced.

One or both of the sacrificial film 158Bf and the mask film 159Bf may be formed using an organic material. For example, as the organic material, a material that can be dissolved in a solvent chemically stable with respect to at least the uppermost film of the organic compound film 103Bf may be used. Specifically, a material that will be dissolved in water or an alcohol can be suitably used. In forming a film of such a material, it is preferable to apply the material dissolved in a solvent such as water or an alcohol by a wet process and then perform heat treatment for evaporating the solvent. At this time, the heat treatment is preferably performed in a reduced-pressure atmosphere, in which case the solvent can be removed at a low temperature in a short time and thermal damage to the organic compound film 103Bf can be reduced accordingly.

The sacrificial film 158Bf and the mask film 159Bf may be formed using an organic resin such as polyvinyl alcohol (PVA), polyvinyl butyral, polyvinylpyrrolidone, polyethylene glycol, polyglycerin, pullulan, water-soluble cellulose, an alcohol-soluble polyamide resin, or a fluorine resin like perfluoropolymer.

For example, an organic film (e.g., a PVA film) formed by an evaporation method or the above wet process can be used as the sacrificial film 158Bf, and an inorganic film (e.g., a silicon nitride film) formed by a sputtering method can be used as the mask film 159Bf.

Subsequently, a resist mask 190B is formed over the mask film 159Bf as illustrated in FIG. 7D. The resist mask 190B can be formed by application of a photosensitive material (photoresist), light exposure, and development.

The resist mask 190B may be formed using either a positive resist material or a negative resist material.

The resist mask 190B is provided at a position overlapping with the conductive layer 152B. The resist mask 190B is preferably provided also at a position overlapping with the conductive layer 152C. This can inhibit the conductive layer 152C from being damaged during the fabrication process of the light-emitting apparatus. Note that the resist mask 190B is not necessarily provided over the conductive layer 152C. The resist mask 190B is preferably provided to cover the area from the edge portion of the organic compound film 103Bf to the edge portion of the conductive layer 152C (the edge portion closer to the organic compound film 103Bf), as illustrated in the cross-sectional view along the line B1-B2 in FIG. 7C.

Next, as illustrated in FIG. 7E, part of the mask film 159Bf is removed using the resist mask 190B, so that the mask layer 159B is formed. The mask layer 159B remains over the conductive layers 152B and 152C. After that, the resist mask 190B is removed. Then, part of the sacrificial film 158Bf is removed using the mask layer 159B as a mask (also referred to as a hard mask), so that the sacrificial layer 158B is formed.

Each of the sacrificial film 158Bf and the mask film 159Bf can be processed by a wet etching method or a dry etching method. The sacrificial film 158Bf and the mask film 159Bf are preferably processed by wet etching.

The use of a wet etching method can reduce damage to the organic compound film 103Bf in processing of the sacrificial film 158Bf and the mask film 159Bf, as compared to the case of using a dry etching method. In the case of using a wet etching method, it is preferable to use a developer, an aqueous solution of tetramethylammonium hydroxide (TMAH), dilute hydrofluoric acid, oxalic acid, phosphoric acid, acetic acid, nitric acid, or a chemical solution containing a mixed solution of any of these acids, for example.

Since the organic compound film 103Bf is not exposed in the processing of the mask film 159Bf, the range of choice for a processing method for the mask film 159Bf is wider than that for the sacrificial film 158Bf. Specifically, even in the case where a gas containing oxygen is used as the etching gas in the processing of the mask film 159Bf, deterioration of the organic compound film 103Bf can be suppressed.

In the case where a wet etching method is employed, it is particularly preferable to use an acidic chemical solution. As an acidic chemical solution, a chemical solution containing one of phosphoric acid, hydrofluoric acid, nitric acid, acetic acid, oxalic acid, sulfuric acid, and the like or a mixed chemical solution (also referred to as a mixed acid) that contains two or more of these acids is preferably used.

In the case of using a dry etching method to process the sacrificial film 158Bf, deterioration of the organic compound film 103Bf can be suppressed by not using a gas containing oxygen as the etching gas. In the case of using a dry etching method, it is preferable to use a gas containing CF₄, C₄F₈, SF₆, CHF₃, Cl₂, H₂O, BCl₃, or a Group 18 element such as He, for example, as the etching gas.

The resist mask 190B can be removed by a method similar to that for the resist mask 191. At this time, the sacrificial film 158Bf is positioned on the outermost surface, and the organic compound film 103Bf is not exposed; thus, the organic compound film 103Bf can be inhibited from being damaged in the step of removing the resist mask 190B. In addition, the range of choice of the method for removing the resist mask 190B can be widened.

Next, as illustrated in FIG. 7E, the organic compound film 103Bf is processed to form the organic compound layer 103B. For example, part of the organic compound film 103Bf is removed using the mask layer 159B and the sacrificial layer 158B as hard masks to form the organic compound layer 103B.

Accordingly, as illustrated in FIG. 7E, the stacked-layer structure of the organic compound layer 103B, the sacrificial layer 158B, and the mask layer 159B remains over the conductive layer 152B. The conductive layers 152G and 152R are exposed.

The organic compound film 103Bf can be processed by dry etching or wet etching. In the case where the processing is performed by a dry etching method, for example, an etching gas containing oxygen can be used. When the etching gas contains oxygen, the etching rate can be increased. Thus, the etching can be performed under a low-power condition while an adequately high etching rate is maintained. Accordingly, damage to the organic compound film 103Bf can be reduced. Furthermore, a defect such as attachment of a reaction product generated during the etching can be reduced.

An etching gas that does not contain oxygen may be used. In that case, deterioration of the organic compound film 103Bf can be suppressed, for example.

As described above, in one embodiment of the present invention, the mask layer 159B is formed in the following manner: the resist mask 190B is formed over the mask film 159Bf and part of the mask film 159Bf is removed with use of the resist mask 190B. After that, part of the organic compound film 103Bf is removed with the mask layer 159B used as a hard mask, so that the organic compound layer 103B is formed. In other words, the organic compound layer 103B can be formed by processing the organic compound film 103Bf by a photolithography method. Note that part of the organic compound film 103Bf may be removed with use of the resist mask 190B. Then, the resist mask 190B may be removed.

Here, hydrophobization treatment for the conductive layer 152G may be performed as necessary. At the time of processing the organic compound film 103Bf, the property of a surface of the conductive layer 152G change to a hydrophilic property in some cases, for example. The hydrophobization treatment for the conductive layer 152G, for example, can increase the adhesion between the conductive layer 152G and a layer to be formed in a later step (which is the organic compound layer 103G here) and suppress film peeling.

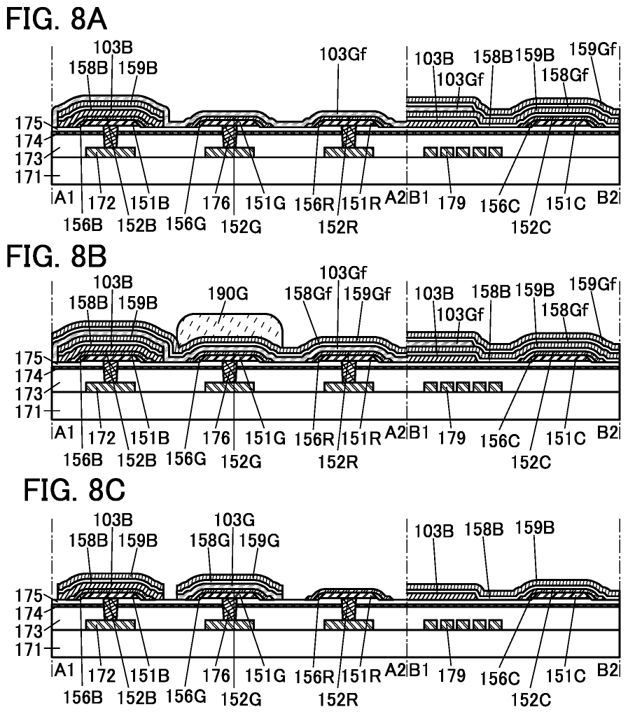

Next, as illustrated in FIG. 8A, an organic compound film 103Gf to be the organic compound layer 103G is formed over the conductive layer 152G, the conductive layer 152R, the mask layer 159B, and the insulating layer 175.

The organic compound film 103Gf can be formed by a method similar to that for forming the organic compound film 103Bf. The organic compound film 103Gf can have a structure similar to that of the organic compound film 103Bf.

Then, as illustrated in FIG. 8B, a sacrificial film 158Gf to be a sacrificial layer 158G and a mask film 159Gf to be a mask layer 159G are sequentially formed over the organic compound film 103Gf and the mask layer 159B. After that, a resist mask 190G is formed. The materials and the formation methods of the sacrificial film 158Gf and the mask film 159Gf are similar to those for the sacrificial film 158Bf and the mask film 159Bf. The material and the formation method of the resist mask 190G are similar to those for the resist mask 190B.

The resist mask 190G is provided at a position overlapping with the conductive layer 152G.

Subsequently, as illustrated in FIG. 8C, part of the mask film 159Gf is removed with use of the resist mask 190G, whereby the mask layer 159G is formed. The mask layer 159G remains over the conductive layer 152G. After that, the resist mask 190G is removed. Then, part of the sacrificial film 158Gf is removed with the mask layer 159G used as a mask, so that the sacrificial layer 158G is formed. Next, the organic compound film 103Gf is processed to form the organic compound layer 103G. For example, part of the organic compound film 103Gf is removed with the mask layer 159G and the sacrificial layer 158G used as hard masks to form the organic compound layer 103G.

Accordingly, as illustrated in FIG. 8C, the stacked-layer structure of the organic compound layer 103G, the sacrificial layer 158G, and the mask layer 159G remains over the conductive layer 152G. The mask layer 159B and the conductive layer 152R are exposed.

Hydrophobization treatment for the conductive layer 152R may be performed, for example.

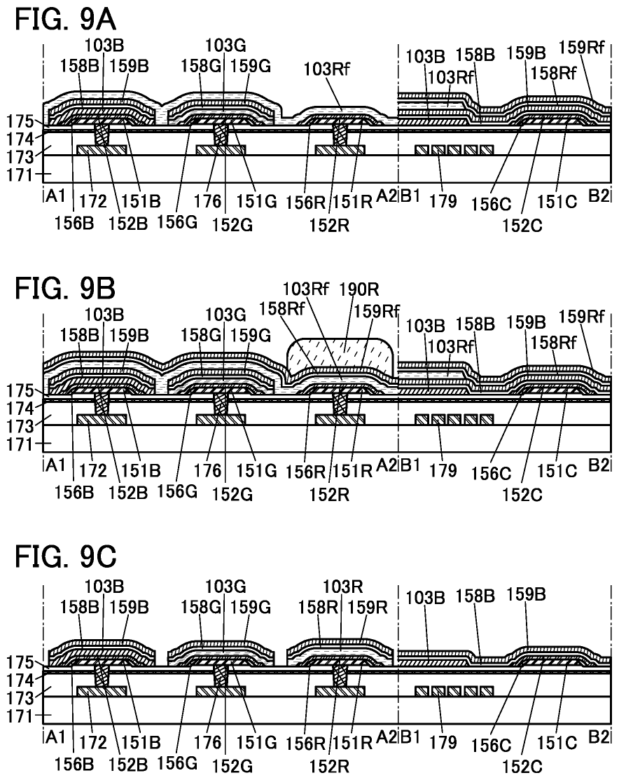

Next, as illustrated in FIG. 9A, an organic compound film 103Rf to be the organic compound layer 103R is formed over the conductive layer 152R, the mask layer 159G, the mask layer 159B, and the insulating layer 175.

The organic compound film 103Rf can be formed by a method similar to that for forming the organic compound film 103Gf. The organic compound film 103Rf can have a structure similar to that of the organic compound film 103Gf.

Subsequently, as illustrated in FIGS. 9B and 9C, a sacrificial layer 158R, a mask layer 159R, and the organic compound layer 103R are formed from a sacrificial film 158Rf, a mask film 159Rf, and the organic compound film 103Rf, respectively. For the formation methods of the sacrificial layer 158R, the mask layer 159R, and the organic compound layer 103R, the description for the organic compound layer 103G can be referred to.

Note that the side surfaces of the organic compound layers 103B, 103G, and 103R are preferably perpendicular or substantially perpendicular to their formation surfaces. For example, the angle between the formation surfaces and these side surfaces is preferably greater than or equal to 60° and less than or equal to 90°.

The distance between two adjacent layers among the organic compound layers 103B, 103G, and 103R, which are formed by a photolithography method as described above, can be reduced to less than or equal to 8 μm, less than or equal to 5 μm, less than or equal to 3 μm, less than or equal to 2 μm, or less than or equal to 1 μm. Here, the distance can be determined, for example, by the distance between opposite edge portions of two adjacent layers among the organic compound layers 103B, 103G, and 103R. Reducing the distance between the island-shaped organic compound layers can provide a light-emitting apparatus having high definition and a high aperture ratio. In addition, the distance between the first electrodes of adjacent light-emitting devices can also be shortened to be, for example, less than or equal to 10 μm, less than or equal to 8 μm, less than or equal to 5 μm, less than or equal to 3 μm, or less than or equal to 2 μm. Note that the distance between the first electrodes of adjacent light-emitting devices is preferably greater than or equal to 2 μm and less than or equal to 5 μm.

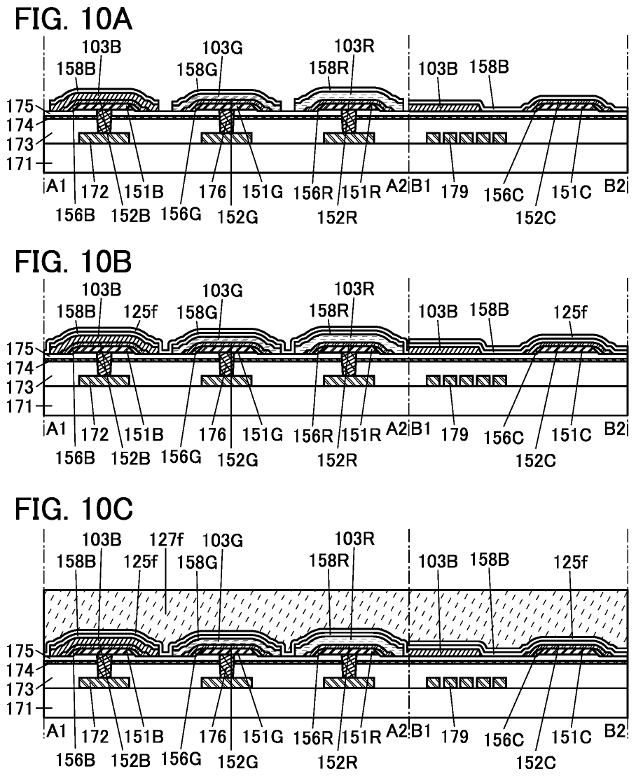

Next, as illustrated in FIG. 10A, the mask layers 159B, 159G, and 159R are removed.

This embodiment describes an example where the mask layers 159B, 159G, and 159R are removed; however, the mask layers 159B, 159G, and 159R may be left without being removed. For example, in the case where the mask layers 159B, 159G, and 159R contain the above-described material having a property of blocking ultraviolet rays, the procedure preferably proceeds to the next step without removing the mask layers 159B, 159G, and 159R, in which case the organic compound layers can be protected from light irradiation (including lighting).

The step of removing the mask layers can be performed by a method similar to that for the step of processing the mask films. Specifically, by using a wet etching method, damage applied to the organic compound layers 103B, 103G, and 103R at the time of removing the mask layers can be reduced as compared to the case of using a dry etching method.

The mask layers may be removed by being dissolved in a solvent such as water or an alcohol. Examples of the alcohol include ethyl alcohol, methyl alcohol, isopropyl alcohol (IPA), and glycerin.

After the mask layers are removed, drying treatment may be performed in order to remove water included in the organic compound layers 103B, 103G, and 103R and water adsorbed on surfaces of the organic compound layers 103B, 103G, and 103R. For example, heat treatment in an inert atmosphere or a reduced-pressure atmosphere can be performed. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 120° C. The heat treatment is preferably performed in a reduced-pressure atmosphere, in which case drying at a lower temperature is possible.

Next, as illustrated in FIG. 10B, the inorganic insulating film 125f to be the inorganic insulating layer 125 is formed to cover the organic compound layers 103B, 103G, and 103R and the sacrificial layers 158B, 158G, and 158R.

As described later, an insulating film to be the insulating layer 127 is formed in contact with the top surface of the inorganic insulating film 125f. Thus, the top surface of the inorganic insulating film 125f preferably has a high affinity for the material used for the insulating film to be the insulating layer 127 (e.g., a photosensitive resin composition containing an acrylic resin). To improve the affinity, surface treatment may be performed on the top surface of the inorganic insulating film 125f. Specifically, a surface of the inorganic insulating film 125f is preferably made hydrophobic (or its hydrophobic property is preferably improved). For example, it is preferable to perform the treatment using a silylation agent such as hexamethyldisilazane (HMDS). By making the top surface of the inorganic insulating film 125f hydrophobic in this manner, an insulating film 127f can be formed to have favorable adhesion.

Then, as illustrated in FIG. 10C, an insulating film 127f to be the insulating layer 127 is formed over the inorganic insulating film 125f.

The inorganic insulating film 125f and the insulating film 127f are preferably formed by a formation method by which the organic compound layers 103B, 103G, and 103R are less damaged. The inorganic insulating film 125f, which is formed in contact with the side surfaces of the organic compound layers 103B, 103G, and 103R, is particularly preferably formed by a formation method that causes less damage to the organic compound layers 103B, 103G, and 103R than the method of forming the insulating film 127f.

Each of the inorganic insulating film 125f and the insulating film 127f is formed at a temperature lower than the upper temperature limit of the organic compound layers 103B, 103G, and 103R. When the inorganic insulating film 125f is formed at a high substrate temperature, the formed inorganic insulating film 125f, even with a small thickness, can have a low impurity concentration and a high barrier property against at least one of water and oxygen.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness greater than or equal to 3 nm, greater than or equal to 5 nm, or greater than or equal to 10 nm and less than or equal to 200 nm, less than or equal to 150 nm, less than or equal to 100 nm, or less than or equal to 50 nm is preferably formed in the above-described range of the substrate temperature.

The inorganic insulating film 125f is preferably formed by an ALD method, for example. An ALD method is preferably used, in which case damage caused by deposition is reduced and a film with good coverage can be formed. As the inorganic insulating film 125f, an aluminum oxide film is preferably formed by an ALD method, for example.

Alternatively, the inorganic insulating film 125f may be formed by a sputtering method, a CVD method, or a PECVD method, each of which has a higher deposition rate than an ALD method. In that case, a highly reliable light-emitting apparatus can be fabricated with high productivity.

The insulating film 127f is preferably formed by the aforementioned wet process. The insulating film 127f is preferably formed by spin coating using a photosensitive material, for example, and preferably formed using specifically a photosensitive resin composition containing an acrylic resin.

The insulating film 127f is preferably formed using a resin composition containing a polymer, an acid-generating agent, and a solvent, for example. The polymer is formed using one or more kinds of monomers and has a structure where one or more kinds of structural units (also referred to as building blocks) are repeated regularly or irregularly. As the acid-generating agent, one or both of a compound that generates an acid by light irradiation and a compound that generates an acid by heating can be used. The resin composition may also include one or more of a photosensitizing agent, a sensitizer, a catalyst, an adhesive aid, a surface-active agent, and an antioxidant.

Heat treatment (also referred to as prebaking) is preferably performed after the insulating film 127f is formed. The heat treatment is performed at a temperature lower than the upper temperature limit of the organic compound layers 103B, 103G, and 103R. The substrate temperature in the heat treatment is preferably higher than or equal to 50° C. and lower than or equal to 200° C., further preferably higher than or equal to 60° C. and lower than or equal to 150° C., still further preferably higher than or equal to 70° C. and lower than or equal to 120° C. Accordingly, the solvent contained in the insulating film 127f can be removed.

Then, part of the insulating film 127f is exposed to visible light or ultraviolet rays. Here, when a positive photosensitive resin composition containing an acrylic resin is used for the insulating film 127f, a region where the insulating layer 127 is not formed in a later step is irradiated with visible light or ultraviolet rays. The insulating layer 127 is formed in regions that are sandwiched between any two of the conductive layers 152B, 152G, and 152R and around the conductive layer 152C. Thus, the top surfaces of the conductive layers 152B, 152G, 152R, and 152C are irradiated with visible light or ultraviolet rays. Note that when a negative photosensitive material is used for the insulating film 127f, the region where the insulating layer 127 is to be formed is irradiated with visible light or ultraviolet rays.

The width of the insulating layer 127 formed later can be controlled in accordance with the exposed region of the insulating film 127f. In this embodiment, processing is performed such that the insulating layer 127 includes a portion overlapping with the top surface of the conductive layer 151.

Here, when a barrier insulating layer against oxygen (e.g., an aluminum oxide film) is provided as one or both of the sacrificial layer 158 (the sacrificial layers 158B, 158G, and 158R) and the inorganic insulating film 125f, diffusion of oxygen to the organic compound layers 103B, 103G, and 103R can be reduced. When the organic compound layer is irradiated with light (visible light or ultraviolet rays), the organic compound contained in the organic compound layer is put into an excited state and a reaction between the organic compound and oxygen in the atmosphere is promoted in some cases. Specifically, when the organic compound layer is irradiated with light (visible light or ultraviolet rays) in an atmosphere containing oxygen, oxygen might be bonded to the organic compound contained in the organic compound layer. By providing the sacrificial layer 158 and the inorganic insulating film 125f over the island-shaped organic compound layer, bonding of oxygen in the atmosphere to the organic compound contained in the organic compound layer can be reduced.

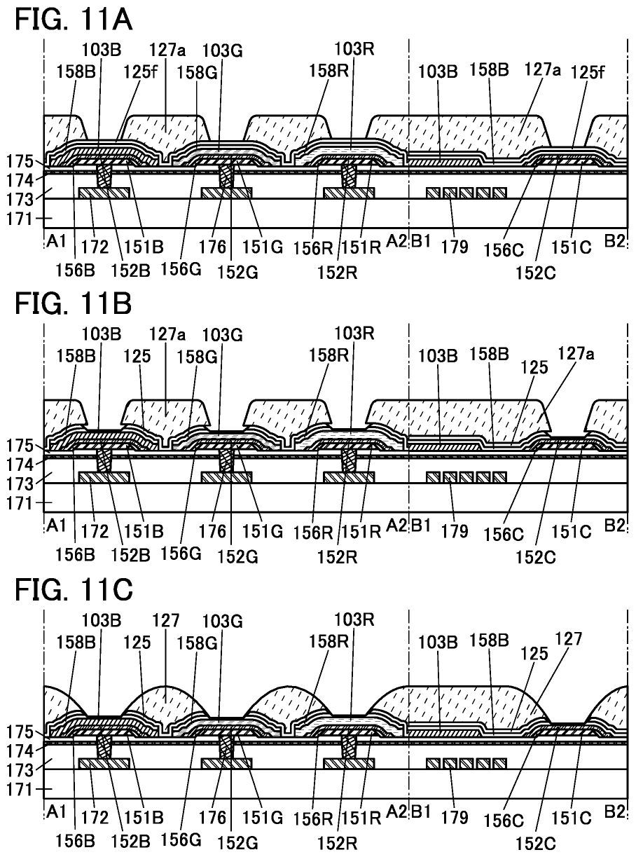

Next, as illustrated in FIG. 11A, development is performed to remove the exposed region of the insulating film 127f, so that an insulating layer 127a is formed. The insulating layer 127a is formed in regions that are sandwiched between any two of the conductive layers 152B, 152G, and 152R and a region surrounding the conductive layer 152C. Here, when an acrylic resin is used for the insulating film 127f, an alkaline solution, such as TMAH, can be used as a developer.

Next, as illustrated in FIG. 11B, etching treatment is performed with the insulating layer 127a as a mask to remove part of the inorganic insulating film 125f and reduce the thicknesses of parts of the sacrificial layers 158B, 158G, and 158R. Thus, the inorganic insulating layer 125 is formed under the insulating layer 127a. Note that the etching treatment for processing the inorganic insulating film 125f with the insulating layer 127a used as a mask may be hereinafter referred to as first etching treatment.

In other words, the sacrificial layers 158B, 158G, and 158R are not removed completely by the first etching treatment, and the etching treatment is stopped when the thicknesses of the sacrificial layers 158B, 158G, and 158R become small. The sacrificial layers 158B, 158G, and 158R are left over the corresponding organic compound layers 103B, 103G, and 103R in this manner, whereby the organic compound layers 103B, 103G, and 103R can be prevented from being damaged by treatment in a later step.

The first etching treatment can be performed by dry etching or wet etching. Note that the inorganic insulating film 125f is preferably formed using a material similar to that of the sacrificial layers 158B, 158G, and 158R, in which case the processing of the inorganic insulating film 125f and thinning of the exposed part of the sacrificial layer 158 can be concurrently performed by the first etching treatment.

By etching using the insulating layer 127a with a tapered side surface used as a mask, the side surface of the inorganic insulating layer 125 and upper edge portions of the side surfaces of the sacrificial layers 158B, 158G, and 158R can be made to have a tapered shape relatively easily.

In the case where the first etching treatment is performed by dry etching, for example, a chlorine-based gas can be used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the thin regions of the sacrificial layers 158B, 158G, and 158R can be formed to have favorable in-plane uniformity.

The first etching treatment can be performed by wet etching, for example. The use of wet etching can reduce damage to the organic compound layers 103B, 103G, and 103R, as compared to the case of using dry etching.

The wet etching is preferably performed using an acidic chemical solution. As an acidic chemical solution, a chemical solution containing one of phosphoric acid, hydrofluoric acid, nitric acid, acetic acid, oxalic acid, sulfuric acid, and the like or a mixed chemical solution (also referred to as a mixed acid) that contains two or more of these acids is preferably used.

The wet etching can be performed using an alkaline solution. For instance, TMAH, which is an alkaline solution, can be used for the wet etching of an aluminum oxide film. In that case, puddle wet etching can be performed.

Then, heat treatment (also referred to as post-baking) is performed. The heat treatment can change the insulating layer 127a into the insulating layer 127 having a tapered side surface (FIG. 11C). The heat treatment is conducted at a temperature lower than the upper temperature limit of the organic compound layer. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 130° C. The heating atmosphere may be an air atmosphere or an inert atmosphere. Moreover, the heating atmosphere may be an atmospheric-pressure atmosphere or a reduced-pressure atmosphere. The substrate temperature in the heat treatment of this step is preferably higher than that in the heat treatment (prebaking) after the formation of the insulating film 127f.

The heat treatment can improve adhesion between the insulating layer 127 and the inorganic insulating layer 125 and increase corrosion resistance of the insulating layer 127. Furthermore, owing to the change in shape of the insulating layer 127a, an edge portion of the inorganic insulating layer 125 can be covered with the insulating layer 127.

When the sacrificial layers 158B, 158G, and 158R are not completely removed by the first etching treatment and the thinned sacrificial layers 158B, 158G, and 158R are left, the organic compound layers 103B, 103G, and 103R can be prevented from being damaged and deteriorating in the heat treatment. This increases the reliability of the light-emitting device.

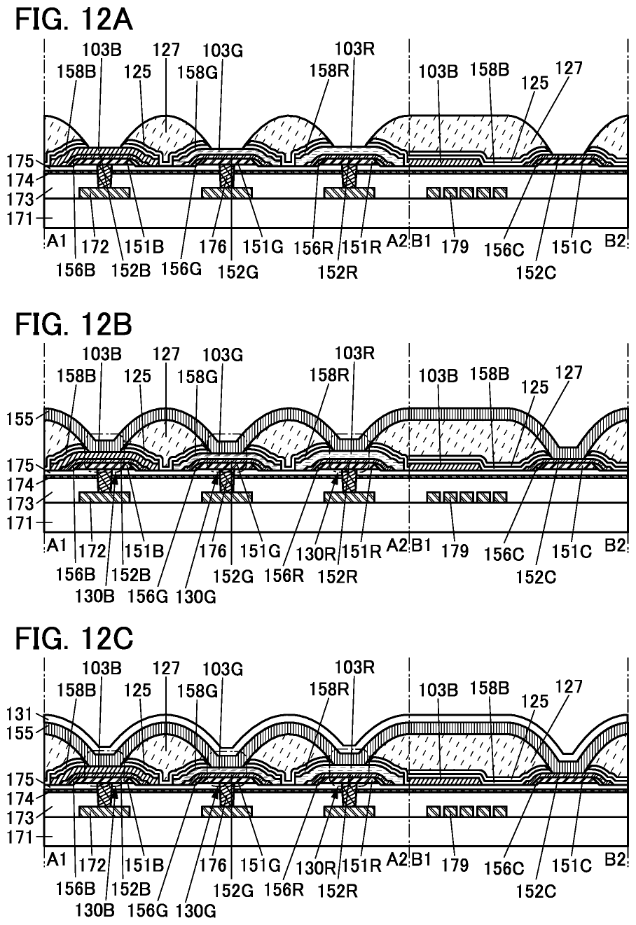

Next, as illustrated in FIG. 12A, etching treatment is performed with the insulating layer 127 used as a mask to remove parts of the sacrificial layers 158B, 158G, and 158R. At this time, part of the inorganic insulating layer 125 is also removed in some cases. By the etching treatment, openings are formed in the sacrificial layers 158B, 158G, and 158R, and the top surfaces of the organic compound layers 103B, 103G, and 103R and the conductive layer 152C are exposed in the openings. Note that the etching treatment for exposing the organic compound layers 103B, 103G, and 103R with the insulating layer 127 used as a mask may be hereinafter referred to as second etching treatment.

The second etching treatment is performed by wet etching. The use of wet etching can reduce damage to the organic compound layers 103B, 103G, and 103R, as compared to the case of using dry etching. The wet etching can be performed using an acidic chemical solution or an alkaline solution as in the first etching treatment.

Heat treatment may be performed after the organic compound layers 103B, 103G, and 103R are partly exposed. By the heat treatment, water included in the organic compound layer and water adsorbed on a surface of the organic compound layer, for example, can be removed. The shape of the insulating layer 127 may be changed by the heat treatment. Specifically, the insulating layer 127 may be widened to cover at least one of the edge portion of the inorganic insulating layer 125, the edge portions of the sacrificial layers 158B, 158G, and 158R, and the top surfaces of the organic compound layers 103B, 103G, and 103R.

FIG. 12A illustrates an example in which part of the edge portion of the sacrificial layer 158 (sacrificial layers 158G, 158B, and 158R) (specifically a tapered portion formed by the first etching treatment) is covered with the insulating layer 127 and a tapered portion formed by the second etching treatment is exposed (see FIG. 5A).

The insulating layer 127 may cover the entire edge portion of the sacrificial layer 158G. For example, an edge portion of the insulating layer 127 may droop to cover the edge portion of the sacrificial layer 158G. For another example, the edge portion of the insulating layer 127 may be in contact with the top surface of at least one of the organic compound layers 103B, 103G, and 103R.

Next, as illustrated in FIG. 12B, a common electrode 155 is formed over the organic compound layers 103B, 103G, and 103R, the conductive layer 152C, and the insulating layer 127. The common electrode 155 can be formed by a sputtering method, a vacuum evaporation method, or the like. Alternatively, the common electrode 155 may be formed by stacking a film formed by an evaporation method and a film formed by a sputtering method.

Next, as illustrated in FIG. 12C, the protective layer 131 is formed over the common electrode 155. The protective layer 131 can be formed by a vacuum evaporation method, a sputtering method, a CVD method, an ALD method, or the like.

Then, the substrate 120 is bonded over the protective layer 131 using the resin layer 122, so that the light-emitting apparatus can be fabricated. In the method for fabricating the light-emitting apparatus of one embodiment of the present invention, the insulating layer 156 is formed to include a region overlapping with the side surface of the conductive layer 151 and the conductive layer 152 is formed to cover the conductive layer 151 and the insulating layer 156 as described above. This can increase the yield of the light-emitting apparatus and suppress generation of defects.

As described above, in the method for fabricating the light-emitting apparatus of one embodiment of the present invention, the island-shaped organic compound layers 103B, 103G, and 103R are formed not by using a fine metal mask but by processing a film formed on the entire surface; thus, the island-shaped layers can be formed to have a uniform thickness. Consequently, a high-definition light-emitting apparatus or a light-emitting apparatus with a high aperture ratio can be obtained. Furthermore, even when the definition or the aperture ratio is high and the distance between the subpixels is extremely short, the organic compound layers 103B, 103G, and 103R can be inhibited from being in contact with each other in the adjacent subpixels. As a result, generation of a leakage current between the subpixels can be suppressed. This can prevent crosstalk, so that a light-emitting apparatus with extremely high contrast can be obtained. Moreover, even a light-emitting apparatus that includes tandem light-emitting devices formed by a photolithography method can have favorable characteristics.

### Embodiment 4

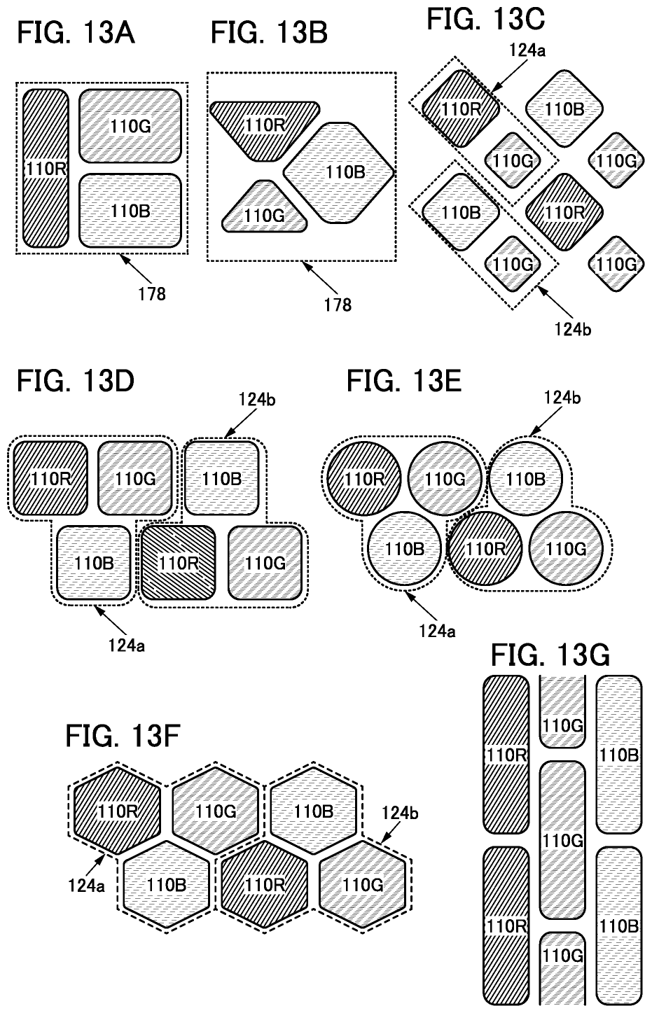

In this embodiment, the light-emitting apparatus of one embodiment of the present invention will be described with reference to FIGS. 13A to 13G and FIGS. 14A to 141.

### [Pixel Layout]

In this embodiment, pixel layouts different from that in FIG. 4A will be mainly described. There is no particular limitation on the subpixel layout, and a variety of methods can be employed. Examples of the subpixel layout include stripe layout, S-stripe layout, matrix layout, delta layout, Bayer layout, and PenTile layout.

In this embodiment, the top surface shapes of the subpixels shown in the diagrams correspond to top surface shapes of light-emitting regions.

Examples of a top surface shape of the subpixel include polygons such as a triangle, a tetragon (including a rectangle and a square), and a pentagon; these polygons with rounded corners; an ellipse; and a circle.

The circuit constituting the subpixel is not necessarily placed within the dimensions of the subpixel illustrated in the diagrams and may be placed outside the subpixel.

The pixel 178 illustrated in FIG. 13A employs S-stripe layout. The pixel 178 illustrated in FIG. 13A includes three subpixels, the subpixel 110R, the subpixel 110G, and the subpixel 110B.

The pixel 178 illustrated in FIG. 13B includes the subpixel 110R whose top surface has a rough trapezoidal shape with rounded corners, the subpixel 110G whose top surface has a rough triangle shape with rounded corners, and the subpixel 110B whose top surface has a rough tetragonal or rough hexagonal shape with rounded corners. The subpixel 110R has a larger light-emitting area than the subpixel 110G. In this manner, the shapes and sizes of the subpixels can be determined independently. For example, the size of a subpixel including a light-emitting device with higher reliability can be smaller.

Pixels 124a and 124b illustrated in FIG. 13C employ PenTile layout. FIG. 13C illustrates an example in which the pixels 124a including the subpixels 110R and 110G and the pixels 124b including the subpixels 110G and 110B are alternately arranged.

The pixels 124a and 124b illustrated in FIGS. 13D to 13F employ delta layout. The pixel 124a includes two subpixels (the subpixels 110R and 110G) in the upper row (first row) and one subpixel (the subpixel 110B) in the lower row (second row). The pixel 124b includes one subpixel (the subpixel 110B) in the upper row (first row) and two subpixels (the subpixels 110R and 110G) in the lower row (second row).

FIG. 13D illustrates an example where the top surface of each subpixel has a rough tetragonal shape with rounded corners. FIG. 13E illustrates an example where the top surface of each subpixel is circular. FIG. 13F illustrates an example where the top surface of each subpixel has a rough hexagonal shape with rounded corners.

In FIG. 13F, subpixels are placed in respective hexagonal regions that are arranged densely. One subpixel of the subpixels is placed so as to be surrounded by six subpixels. The subpixels are arranged such that subpixels that emit light of the same color are not adjacent to each other. For example, one subpixel 110R is surrounded by three subpixels 110G and three subpixels 110B that are alternately arranged.

FIG. 13G illustrates an example where subpixels of different colors are arranged in a zigzag manner. Specifically, the positions of the top sides of two subpixels arranged in the row direction (e.g., the subpixels 110R and 110G or the subpixels 110G and 1101B) are not aligned in the top view.

In the pixels illustrated in FIGS. 13A to 13G, for example, it is preferable that the subpixel 110R be a subpixel R that emits red light, the subpixel 110G be a subpixel G that emits green light, and the subpixel 110B be a subpixel B that emits blue light. Note that the structures of the subpixels are not limited thereto, and the colors and the order of the subpixels can be determined as appropriate. For example, the subpixel 110G may be the subpixel R that emits red light, and the subpixel 110R may be the subpixel G that emits green light.

In a photolithography method, as a pattern to be formed by processing becomes finer, the influence of light diffraction becomes more difficult to ignore; therefore, the fidelity in transferring a photomask pattern by light exposure is degraded, and it becomes difficult to process a resist mask into a desired shape. Thus, a pattern with rounded corners is likely to be formed even with a rectangular photomask pattern. Consequently, the top surface of a subpixel may have a polygonal shape with rounded corners, an elliptical shape, a circular shape, or the like.

Furthermore, in the method for fabricating the light-emitting apparatus of one embodiment of the present invention, the organic compound layer is processed into an island shape with the use of a resist mask. A resist film formed over the organic compound layer needs to be cured at a temperature lower than the upper temperature limit of the organic compound layer. Therefore, the resist film is insufficiently cured in some cases depending on the upper temperature limit of the material of the organic compound layer and the curing temperature of the resist material. An insufficiently cured resist film may have a shape different from a desired shape by processing. As a result, the top surface of the organic compound layer may have a polygonal shape with rounded corners, an elliptical shape, a circular shape, or the like. For example, when a resist mask with a square top surface is intended to be formed, a resist mask with a circular top surface may be formed, and the top surface of the organic compound layer may be circular.

To obtain a desired top surface shape of the organic compound layer, a technique of correcting a mask pattern in advance so that a transferred pattern agrees with a design pattern (an optical proximity correction (OPC) technique) may be used. Specifically, with the OPC technique, a pattern for correction is added to a corner portion of a figure on a mask pattern, for example.

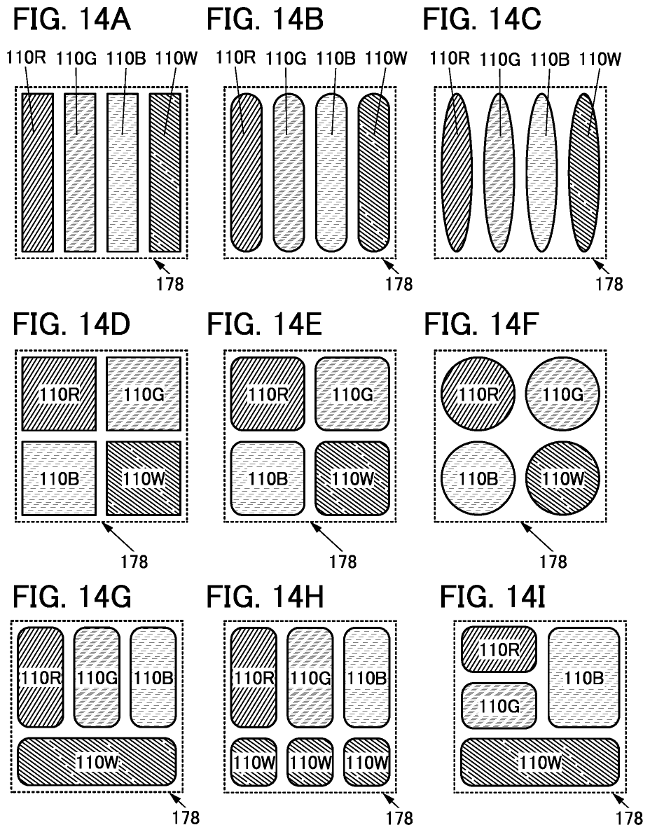

As illustrated in FIGS. 14A to 141, the pixel can include four types of subpixels.

The pixels 178 illustrated in FIGS. 14A to 14C employ stripe layout.

FIG. 14A illustrates an example where each subpixel has a rectangular top surface shape. FIG. 14B illustrates an example where each subpixel has a top surface shape formed by combining two half circles and a rectangle. FIG. 14C illustrates an example where each subpixel has an elliptical top surface shape.

The pixels 178 illustrated in FIGS. 14D to 14F employ matrix layout.

FIG. 14D illustrates an example where each subpixel has a square top surface shape. FIG. 14E illustrates an example where each subpixel has a substantially square top surface shape with rounded corners. FIG. 14F illustrates an example where each subpixel has a circular top surface shape.

FIGS. 14G and 14H each illustrate an example where one pixel 178 is composed of two rows and three columns.

The pixel 178 illustrated in FIG. 14G includes three subpixels (the subpixels 110R, 110G, and 110B) in the upper row (first row) and one subpixel (a subpixel 110W) in the lower row (second row). In other words, the pixel 178 includes the subpixel 110R in the left column (first column), the subpixel 110G in the middle column (second column), the subpixel 110B in the right column (third column), and the subpixel 110W across these three columns.

The pixel 178 illustrated in FIG. 14H includes three subpixels (the subpixels 110R, 110G, and 110B) in the upper row (first row) and three of the subpixels 110W in the lower row (second row). In other words, the pixel 178 includes the subpixels 110R and 110W in the left column (first column), the subpixels 110G and 110W in the middle column (second column), and the subpixels 110B and 110W in the right column (third column). Matching the positions of the subpixels in the upper row and the lower row as illustrated in FIG. 14H enables dust that would be produced in the fabrication process, for example, to be removed efficiently. Thus, a light-emitting apparatus having high display quality can be provided.

In the pixel 178 illustrated in FIGS. 14G and 14H, the subpixels 110R, 110G, and 110B are arranged in a stripe layout, whereby the display quality can be improved.

FIG. 14I illustrates an example where one pixel 178 is composed of three rows and two columns.

The pixel 178 illustrated in FIG. 14I includes the subpixel 110R in the upper row (first row), the subpixel 110G in the middle row (second row), the subpixel 110B across the first row and the second row, and one subpixel (the subpixel 110W) in the lower row (third row). In other words, the pixel 178 includes the subpixels 110R and 110G in the left column (first column), the subpixel 110B in the right column (second column), and the subpixel 110W across these two columns.

In the pixel 178 illustrated in FIG. 14I, the subpixels 110R, 110G, and 110B are arranged in what is called an S-stripe layout, whereby the display quality can be improved.

The pixel 178 illustrated in each of FIGS. 14A to 141 is composed of four subpixels, which are the subpixels 110R, 110G, 110B, and 110W. For example, the subpixel 110R can be a subpixel that emits red light, the subpixel 110G can be a subpixel that emits green light, the subpixel 110B can be a subpixel that emits blue light, and the subpixel 110W can be a subpixel that emits white light. Note that at least one of the subpixels 110R, 110G, 110B, and 110W may be a subpixel that emits cyan light, magenta light, yellow light, or near-infrared light.

As described above, the pixel composed of the subpixels each including the light-emitting device can employ any of a variety of layouts in the light-emitting apparatus of one embodiment of the present invention.

This embodiment can be combined as appropriate with any of the other embodiments and examples. In this specification, in the case where a plurality of structure examples are shown in one embodiment, the structure examples can be combined as appropriate.

### Embodiment 5

In this embodiment, a light-emitting apparatus of one embodiment of the present invention will be described.

The light-emitting apparatus in this embodiment can be a high-definition light-emitting apparatus. Thus, the light-emitting apparatus in this embodiment can be used for display portions of information terminals (wearable devices) such as watch-type and bracelet-type information terminals and display portions of wearable devices capable of being worn on a head, such as a virtual reality (VR) device like a head mounted display (HMD) and a glasses-type augmented reality (AR) device.

The light-emitting apparatus in this embodiment can be a high-resolution light-emitting apparatus or a large-sized light-emitting apparatus. Accordingly, the light-emitting apparatus in this embodiment can be used for display portions of a digital camera, a digital video camera, a digital photo frame, a mobile phone, a portable game console, a portable information terminal, and an audio reproducing device, in addition to display portions of electronic appliances with a relatively large screen, such as a television device, desktop and notebook personal computers, a monitor of a computer and the like, digital signage, and a large game machine such as a pachinko machine.

### [Display Module]

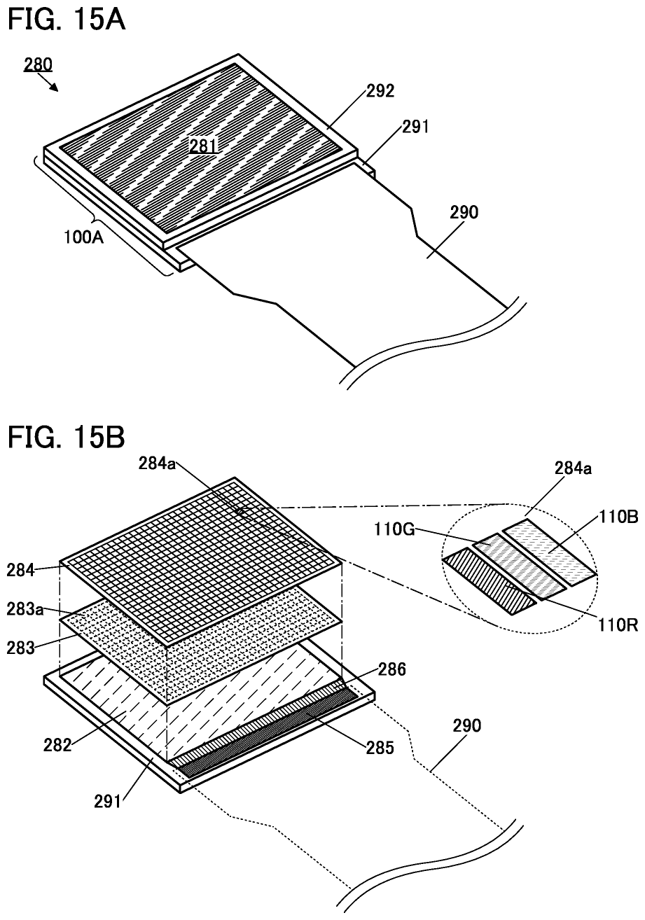

FIG. 15A is a perspective view of a display module 280. The display module 280 includes a light-emitting apparatus 100A and an FPC 290. Note that the light-emitting apparatus included in the display module 280 is not limited to the light-emitting apparatus 100A and may be any of light-emitting apparatuses 100B and 100C described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 15B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion not overlapping with the pixel portion 284 over the substrate 291. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 15B. The pixels 284a can employ any of the structures described in the above embodiments. FIG. 15B illustrates an example where the pixel 284a has a structure similar to that of the pixel 178 illustrated in FIGS. 4A and 4B.

The pixel circuit portion 283 includes a plurality of pixel circuits 283a arranged periodically.

One pixel circuit 283a is a circuit that controls driving of a plurality of elements included in one pixel 284a. One pixel circuit 283a can be provided with three circuits each of which controls light emission of one light-emitting device. For example, the pixel circuit 283a can include at least one selection transistor, one current control transistor (driving transistor), and a capacitor per light-emitting device. A gate signal is input to a gate of the selection transistor, and a video signal is input to a source or a drain of the selection transistor. With such a structure, an active-matrix light-emitting apparatus is obtained.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be higher than or equal to 40% and lower than 100%, preferably higher than or equal to 50% and lower than or equal to 95%, further preferably higher than or equal to 60% and lower than or equal to 95%. Furthermore, the pixels 284a can be arranged extremely densely and thus the display portion 281 can have significantly high definition. For example, the pixels 284a are preferably arranged in the display portion 281 with a pixel density higher than or equal to 2000 ppi, further preferably higher than or equal to 3000 ppi, still further preferably higher than or equal to 5000 ppi, yet still further preferably higher than or equal to 6000 ppi, and lower than or equal to 20000 ppi or lower than or equal to 30000 ppi.

Such a display module 280 has extremely high definition, and thus can be suitably used for a VR device such as an HMD or a glasses-type AR device. For example, even in the case of a structure in which the display portion of the display module 280 is seen through a lens, pixels of the extremely-high-definition display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion. For example, the display module 280 can be favorably used in a display portion of a wearable electronic appliance, such as a wrist watch.

### [Light-Emitting Apparatus 100A]

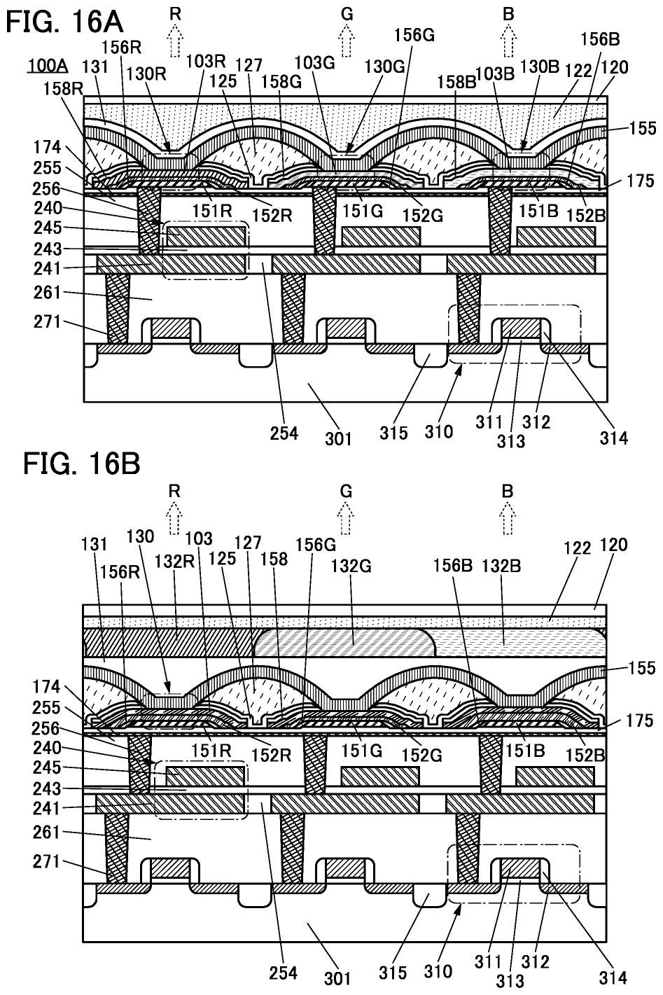

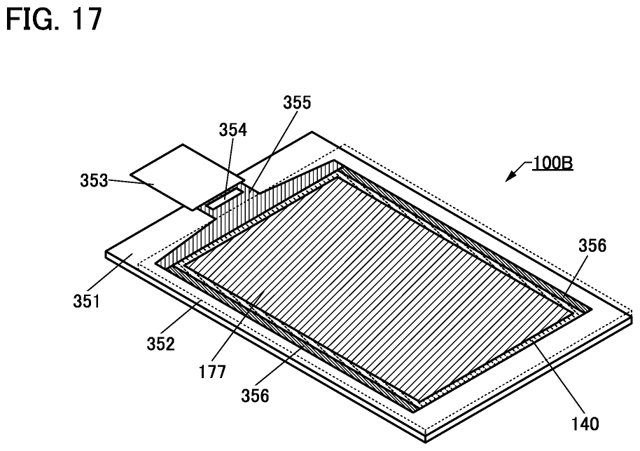

The light-emitting apparatus 100A illustrated in FIG. 16A includes a substrate 301, the light-emitting devices 130R, 130G, and 130B, a capacitor 240, and a transistor 310.

The substrate 301 corresponds to the substrate 291 in FIGS. 15A and 15B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. FIG. 16A illustrates an example in which the light-emitting devices 130R, 130G, and 130B each have the stacked-layer structure illustrated in FIG. 1A. An insulator is provided in regions between adjacent light-emitting devices. For example, in FIG. 16A, the inorganic insulating layer 125 and the insulating layer 127 over the inorganic insulating layer 125 are provided in those regions.

The insulating layer 156R is provided to include a region overlapping with the side surface of the conductive layer 151R of the light-emitting device 130R. The insulating layer 156G is provided to include a region overlapping with the side surface of the conductive layer 151G of the light-emitting device 130G. The insulating layer 156B is provided to include a region overlapping with the side surface of the conductive layer 151B of the light-emitting device 130B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. The sacrificial layer 158R is positioned over the organic compound layer 103R of the light-emitting device 130R. The sacrificial layer 158G is positioned over the organic compound layer 103G of the light-emitting device 130G. The sacrificial layer 158B is positioned over the organic compound layer 103B of the light-emitting device 130B.

The conductive layers 151R, 151G, and 151B is electrically connected to the sources or the drains of the corresponding transistors 310 through plugs 256 embedded in the insulating layers 243, 255, 174, and 175, the conductive layers 241 embedded in the insulating layer 254, and the plugs 271 embedded in the insulating layer 261. The top surface of the insulating layer 175 and the top surface of the plug 256 are level with or substantially level with each other. Any of a variety of conductive materials can be used for the plugs.

The protective layer 131 is provided over the light-emitting devices 130R, 130G, and 130B. The substrate 120 is bonded to the protective layer 131 with the resin layer 122. Embodiment 2 can be referred to for the details of the light-emitting device 130 and the components thereover up to the substrate 120. The substrate 120 corresponds to the substrate 292 in FIG. 15A.

FIG. 16B illustrates a variation example of the light-emitting apparatus 100A illustrated in FIG. 16A. The light-emitting apparatus illustrated in FIG. 16B includes the coloring layers 132R, 132G, and 132B, and each of the light-emitting devices 130 includes a region overlapping with one of the coloring layers 132R, 132G, and 132B. In the light-emitting apparatus illustrated in FIG. 16B, the light-emitting device 130 can emit white light, for example. For example, the coloring layer 132R can transmit red light, the coloring layer 132G can transmit green light, and the coloring layer 132B can transmit blue light.

### [Light-Emitting Apparatus 100B]

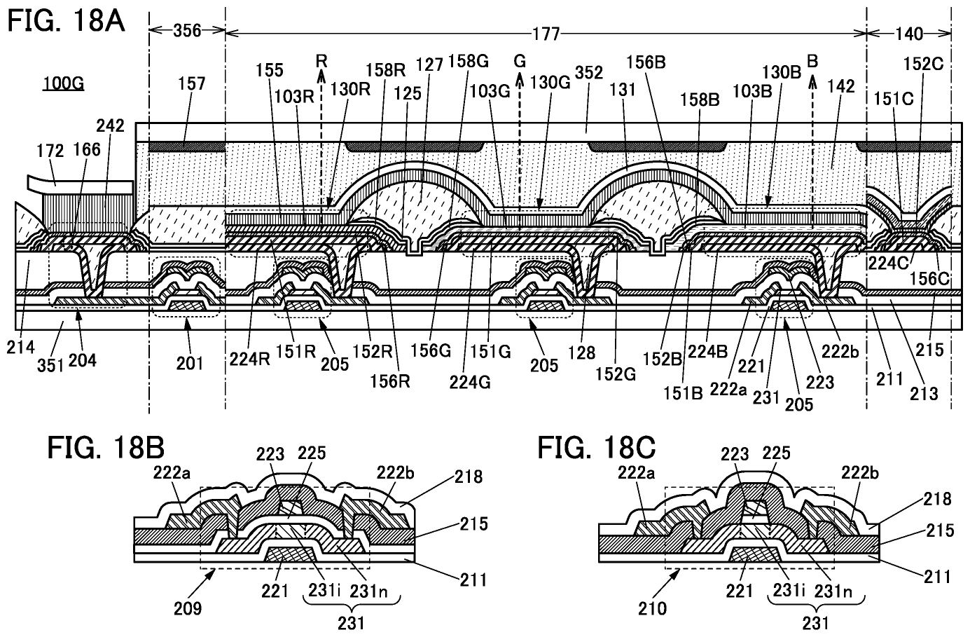

FIG. 17 is a perspective view of the light-emitting apparatus 100B, and FIG. 18A is a cross-sectional view of a light-emitting apparatus 100G, which is an example of the light-emitting apparatus 100n.

In the light-emitting apparatus 100B, a substrate 352 and a substrate 351 are bonded to each other. In FIG. 17, the substrate 352 is denoted by a dashed line.

The light-emitting apparatus 100B includes the pixel portion 177, the connection portion 140, a circuit 356, a wiring 355, and the like. FIG. 17 illustrates an example in which an IC 354 and an FPC 353 are mounted on the light-emitting apparatus 100B. Thus, the structure illustrated in FIG. 17 can be regarded as a display module including the light-emitting apparatus 100B, the integrated circuit (IC), and the FPC. Here, a light-emitting apparatus in which a substrate is equipped with a connector such as an FPC or mounted with an IC is referred to as a display module.

The connection portion 140 is provided outside the pixel portion 177. The connection portion 140 can be provided along one side or a plurality of sides of the pixel portion 177. The number of connection portions 140 may be one or more. FIG. 17 illustrates an example in which the connection portion 140 is provided to surround the four sides of a display portion. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 17 illustrates an example in which the IC 354 is provided for the substrate 351 by a chip on glass (COG) method, a chip on film (COF) method, or the like. An IC including a scan line driver circuit, a signal line driver circuit, or the like can be used as the IC 354, for example. Note that the light-emitting apparatus 100B and the display module are not necessarily provided with an IC. Alternatively, the IC may be mounted on the FPC by a COF method, for example.

FIG. 18A illustrates an example of cross sections of part of a region including the FPC 353, part of the circuit 356, part of the pixel portion 177, part of the connection portion 140, and part of a region including an edge portion of the light-emitting apparatus 100B.

The light-emitting apparatus 100B illustrated in FIG. 18A includes a transistor 201, a transistor 205, the light-emitting device 130R that emits red light, the light-emitting device 130G that emits green light, the light-emitting device 130B that emits blue light, and the like between the substrate 351 and the substrate 352.

The stacked-layer structure of each of the light-emitting devices 130R, 130G, and 130B is the same as that illustrated in FIG. 1A except for the structure of the pixel electrode. Embodiments 1 to 3 can be referred to for the details of the light-emitting devices.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B. Here, the conductive layers 224R, 151R, and 152R can be collectively referred to as the pixel electrode of the light-emitting device 130R; the conductive layers 151R and 152R excluding the conductive layer 224R can also be referred to as the pixel electrode of the light-emitting device 130R. Similarly, the conductive layers 224G, 151G, and 152G can be collectively referred to as the pixel electrode of the light-emitting device 130G; the conductive layers 151G and 152G excluding the conductive layer 224G can also be referred to as the pixel electrode of the light-emitting device 130G. The conductive layers 224B, 151B, and 152B can be collectively referred to as the pixel electrode of the light-emitting device 130B; the conductive layers 151B and 152B excluding the conductive layer 224B can also be referred to as the pixel electrode of the light-emitting device 130B.

The conductive layer 224R is connected to a conductive layer 222b included in the transistor 205 through the opening provided in an insulating layer 214. The edge portion of the conductive layer 151R is positioned outward from an edge portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G and the insulating layer 156G in the light-emitting device 130G are not described in detail because they are respectively similar to the conductive layers 224R, 151R, and 152R and the insulating layer 156R in the light-emitting device 130R; the same applies to the conductive layers 224B, 151B, and 152B and the insulating layer 156B in the light-emitting device 130B.

The conductive layers 224R, 224G, and 224B each have a depression portion covering an opening provided in the insulating layer 214. A layer 128 is embedded in the depression portion.

The layer 128 has a function of filling the depression portions of the conductive layers 224R, 224G, and 224B to enable planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depression portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 131 is provided over the light-emitting devices 130R, 130G, and 130B. The protective layer 131 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 18A, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided not to overlap with the light-emitting device. Alternatively, the space may be filled with a resin other than the frame-shaped adhesive layer 142.

FIG. 18A illustrates an example in which the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and the conductive layer 152C obtained by processing the same conductive film as the conductive layers 152R, 152G, and 152B. In the example illustrated in FIG. 18A, the insulating layer 156C is provided to include a region overlapping with the side surface of the conductive layer 151C.

The light-emitting apparatus 100B has a top-emission structure. Light from the light-emitting device is emitted toward the substrate 352. For the substrate 352, a material having a high visible-light-transmitting property is preferably used. The pixel electrode contains a material that reflects visible light, and the counter electrode (the common electrode 155) contains a material that transmits visible light.

The transistor 201 and the transistor 205 are formed over the substrate 351. These transistors can be fabricated using the same materials in the same steps.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or two or more.

A material through which impurities such as water and hydrogen do not easily diffuse is preferably used for at least one of the insulating layers covering the transistors. This is because such an insulating layer can function as a barrier layer. Such a structure can effectively reduce diffusion of impurities to the transistors from the outside and increase the reliability of the light-emitting apparatus.

An inorganic insulating film is preferably used as each of the insulating layers 211, 213, and 215. As the inorganic insulating film, a silicon nitride film, a silicon oxynitride film, a silicon oxide film, a silicon nitride oxide film, an aluminum oxide film, or an aluminum nitride film can be used, for example. A hafnium oxide film, an yttrium oxide film, a zirconium oxide film, a gallium oxide film, a tantalum oxide film, a magnesium oxide film, a lanthanum oxide film, a cerium oxide film, a neodymium oxide film, or the like may be used. Two or more of the above insulating films may also be stacked.

An organic insulating layer is suitable for the insulating layer 214 functioning as a planarization layer. Examples of materials that can be used for the organic insulating layer include an acrylic resin, a polyimide resin, an epoxy resin, a polyamide resin, a polyimide-amide resin, a siloxane resin, a benzocyclobutene-based resin, a phenol resin, and precursors of these resins. The insulating layer 214 may have a stacked-layer structure of an organic insulating layer and an inorganic insulating layer. The outermost layer of the insulating layer 214 preferably functions as an etching protective layer. This can inhibit formation of a recessed portion in the insulating layer 214 at the time of processing of the conductive layer 224R, 151R, or 152R or the like. Alternatively, a recessed portion may be provided in the insulating layer 214 at the time of processing of the conductive layer 224R, 151R, or 152R or the like.

Each of the transistors 201 and 205 includes a conductive layer 221 functioning as a gate, the insulating layer 211 functioning as a gate insulating layer, a conductive layer 222a and a conductive layer 222b functioning as a source and a drain, a semiconductor layer 231, the insulating layer 213 functioning as a gate insulating layer, and a conductive layer 223 functioning as a gate. Here, a plurality of layers obtained by processing the same conductive film are shown with the same hatching pattern. The insulating layer 211 is positioned between the conductive layer 221 and the semiconductor layer 231. The insulating layer 213 is positioned between the conductive layer 223 and the semiconductor layer 231.

There is no particular limitation on the structure of the transistors included in the light-emitting apparatus of this embodiment. For example, a planar transistor, a staggered transistor, or an inverted staggered transistor can be used. A top-gate transistor or a bottom-gate transistor can be used. Alternatively, gates may be provided above and below a semiconductor layer where a channel is formed.

The structure in which the semiconductor layer where a channel is formed is provided between two gates is employed for each of the transistors 201 and 205. The two gates may be connected to each other and supplied with the same signal to drive the transistor. Alternatively, the threshold voltage of the transistor may be controlled by applying a potential for controlling the threshold voltage to one of the two gates and a potential for driving to the other of the two gates.

There is no particular limitation on the crystallinity of a semiconductor material used for the transistors, and either an amorphous semiconductor or a semiconductor having crystallinity (a microcrystalline semiconductor, a polycrystalline semiconductor, a single crystal semiconductor, or a semiconductor partly including crystal regions) can be used. A semiconductor having crystallinity is preferably used, in which case deterioration of transistor characteristics can be suppressed.

The semiconductor layer of the transistor preferably includes a metal oxide. That is, a transistor including a metal oxide in its channel formation region (hereinafter, also referred to as an OS transistor) is preferably used in the light-emitting apparatus of this embodiment.

Examples of an oxide semiconductor having crystallinity include a c-axis-aligned crystalline oxide semiconductor (CAAC-OS) and a nanocrystalline oxide semiconductor (nc-OS).

Alternatively, a transistor including silicon in its channel formation region (a Si transistor) may be used. Examples of silicon include single crystal silicon, polycrystalline silicon, and amorphous silicon. In particular, a transistor containing low-temperature polysilicon (LTPS) in its semiconductor layer (hereinafter also referred to as an LTPS transistor) can be used. The LTPS transistor has high field-effect mobility and excellent frequency characteristics.

With the use of Si transistors such as LTPS transistors, a circuit required to be driven at a high frequency (e.g., a source driver circuit) can be formed on the same substrate as the display portion. This allows for simplification of an external circuit mounted on the light-emitting apparatus and a reduction in costs of parts and mounting costs.

An OS transistor has much higher field-effect mobility than a transistor including amorphous silicon. In addition, the OS transistor has an extremely low leakage current between a source and a drain in an off state (hereinafter also referred to as an off-state current), and electric charge accumulated in a capacitor that is connected in series to the transistor can be held for a long period. Furthermore, the light-emitting apparatus can consume less power by including the OS transistor.

To increase the luminance of the light-emitting device included in the pixel circuit, the amount of current fed through the light-emitting device needs to be increased. To increase the current amount, the source-drain voltage of a driving transistor included in the pixel circuit needs to be increased. An OS transistor has a higher withstand voltage between a source and a drain than a Si transistor; hence, a high voltage can be applied between the source and the drain of the OS transistor. Therefore, when an OS transistor is used as the driving transistor in the pixel circuit, the amount of current flowing through the light-emitting device can be increased, so that the luminance of the light-emitting device can be increased.

When transistors operate in a saturation region, a change in a source-drain current relative to a change in a gate-source voltage can be smaller in an OS transistor than in a Si transistor. Accordingly, when an OS transistor is used as the driving transistor in the pixel circuit, a current flowing between the source and the drain can be set minutely by a change in a gate-source voltage; hence, the amount of current flowing through the light-emitting device can be controlled. Consequently, the number of gray levels expressed by the pixel circuit can be increased.

Regarding saturation characteristics of a current flowing when transistors operate in a saturation region, even in the case where the source-drain voltage of an OS transistor increases gradually, a more stable current (saturation current) can be fed through the OS transistor than through a Si transistor. Thus, by using an OS transistor as the driving transistor, a stable current can be fed through light-emitting devices even when the current-voltage characteristics of the light-emitting devices vary, for example. In other words, when the OS transistor operates in the saturation region, the source-drain current hardly changes with an increase in the source-drain voltage; hence, the luminance of the light-emitting device can be stable.

As described above, by using OS transistors as the driving transistors included in the pixel circuits, it is possible to suppress black-level degradation, increase the luminance, increase the number of gray levels, and suppress variations in light-emitting devices, for example.

The semiconductor layer preferably contains indium, M (M is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more of aluminum, gallium, yttrium, and tin.

It is particularly preferable that an oxide containing indium (In), gallium (Ga), and zinc (Zn) (also referred to as IGZO) be used for the semiconductor layer. It is preferable to use an oxide containing indium, tin, and zinc. It is preferable to use an oxide containing indium, gallium, tin, and zinc. It is preferable to use an oxide containing indium (In), aluminum (Al), and zinc (Zn) (also referred to as IAZO). It is preferable to use an oxide containing indium (In), aluminum (Al), gallium (Ga), and zinc (Zn) (also referred to as IAGZO).

When the semiconductor layer is an In-M-Zn oxide, the atomic proportion of In is preferably higher than or equal to the atomic proportion of M in the In-M-Zn oxide. Examples of the atomic ratio of the metal elements in such an In-M-Zn oxide are In:M:Zn=1:1:1, 1:1:1.2, 2:1:3, 3:1:2, 4:2:3, 4:2:4.1, 5:1:3, 5:1:6, 5:1:7, 5:1:8, 6:1:6, and 5:2:5 and a composition in the neighborhood of any of the above atomic ratios. Note that the neighborhood of the atomic ratio includes ±30% of an intended atomic ratio.

For example, in the case of describing an atomic ratio of In:Ga:Zn=4:2:3 or a composition in the neighborhood thereof, the case is included in which with the atomic proportion of In being 4, the atomic proportion of Ga is greater than or equal to 1 and less than or equal to 3 and the atomic proportion of Zn is greater than or equal to 2 and less than or equal to 4. In the case of describing an atomic ratio of In:Ga:Zn=5:1:6 or a composition in the neighborhood thereof, the case is included in which with the atomic proportion of In being 5, the atomic proportion of Ga is greater than 0.1 and less than or equal to 2 and the atomic proportion of Zn is greater than or equal to 5 and less than or equal to 7. In the case of describing an atomic ratio of In:Ga:Zn=1:1:1 or a composition in the neighborhood thereof, the case is included in which with the atomic proportion of In being 1, the atomic proportion of Ga is greater than 0.1 and less than or equal to 2 and the atomic proportion of Zn is greater than 0.1 and less than or equal to 2.

The transistors included in the circuit 356 and the transistors included in the pixel portion 177 may have the same structure or different structures. One structure or two or more kinds of structures may be employed for a plurality of transistors included in the circuit 356. Similarly, one structure or two or more kinds of structures may be employed for a plurality of transistors included in the pixel portion 177.

All transistors included in the pixel portion 177 may be OS transistors, or all transistors included in the pixel portion 177 may be Si transistors. Alternatively, some of the transistors included in the pixel portion 177 may be OS transistors and the others may be Si transistors.

For example, when both an LTPS transistor and an OS transistor are used in the pixel portion 177, the light-emitting apparatus can have low power consumption and high driving capability. Note that a structure in which an LTPS transistor and an OS transistor are used in combination is referred to as LTPO in some cases. For example, it is preferable that an OS transistor be used as a transistor functioning as a switch for controlling electrical continuity between wirings and an LTPS transistor be used as a transistor for controlling a current.

For example, one transistor included in the pixel portion 177 functions as a transistor for controlling a current flowing through the light-emitting device and can be referred to as a driving transistor. One of a source and a drain of the driving transistor is electrically connected to the pixel electrode of the light-emitting device. An LTPS transistor is preferably used as the driving transistor. In that case, the amount of current flowing through the light-emitting device can be increased in the pixel circuit.

Another transistor included in the pixel portion 177 functions as a switch for controlling selection or non-selection of a pixel and can be referred to as a selection transistor. A gate of the selection transistor is electrically connected to a gate line, and one of a source and a drain thereof is electrically connected to a source line (signal line). An OS transistor is preferably used as the selection transistor. In that case, the gray level of the pixel can be maintained even with an extremely low frame frequency (e.g., lower than or equal to 1 fps); thus, power consumption can be reduced by stopping the driver in displaying a still image.

As described above, the light-emitting apparatus of one embodiment of the present invention can have all of a high aperture ratio, high definition, high display quality, and low power consumption.

Note that the light-emitting apparatus of one embodiment of the present invention has a structure including the OS transistor and the light-emitting device having a metal maskless (MML) structure. This structure can significantly reduce a leakage current that would flow through a transistor and a leakage current that would flow between adjacent light-emitting devices (sometimes referred to as a horizontal leakage current or a lateral leakage current). Displaying images on the light-emitting apparatus having this structure can bring one or more of image crispness, image sharpness, high color saturation, and a high contrast ratio to the viewer. When a leakage current that would flow through the transistor and a lateral leakage current that would flow between the light-emitting devices are extremely low, leakage of light at the time of black display (black-level degradation) or the like can be minimized.

In particular, in the case where a light-emitting device having an MML structure employs a side-by-side (SBS) structure described above, a layer provided between light-emitting devices (for example, also referred to as an organic layer or a common layer shared by the light-emitting devices) is disconnected; accordingly, side leakage can be prevented or be minimized.

FIGS. 18B and 18C illustrate other structure examples of transistors.

Transistors 209 and 210 each include the conductive layer 221 functioning as a gate, the insulating layer 211 functioning as a gate insulating layer, the semiconductor layer 231 including a channel formation region 231i and a pair of low-resistance regions 231n, the conductive layer 222a connected to one of the pair of low-resistance regions 231n, the conductive layer 222b connected to the other of the pair of low-resistance regions 231n, an insulating layer 225 functioning as a gate insulating layer, the conductive layer 223 functioning as a gate, and the insulating layer 215 covering the conductive layer 223. The insulating layer 211 is positioned between the conductive layer 221 and the channel formation region 231i. The insulating layer 225 is positioned at least between the conductive layer 223 and the channel formation region 231i. Furthermore, an insulating layer 218 covering the transistor may be provided.

FIG. 18B illustrates an example of the transistor 209 in which the insulating layer 225 covers the top and side surfaces of the semiconductor layer 231. The conductive layer 222a and the conductive layer 222b are connected to the corresponding low-resistance regions 231n through openings provided in the insulating layer 225 and the insulating layer 215. One of the conductive layers 222a and 222b functions as a source, and the other functions as a drain.

In the transistor 210 illustrated in FIG. 18C, the insulating layer 225 overlaps with the channel formation region 231i of the semiconductor layer 231 and does not overlap with the low-resistance regions 231n. The structure illustrated in FIG. 18C can be obtained by processing the insulating layer 225 with the conductive layer 223 used as a mask, for example. In FIG. 18C, the insulating layer 215 is provided to cover the insulating layer 225 and the conductive layer 223, and the conductive layer 222a and the conductive layer 222b are connected to the corresponding low-resistance regions 231n through openings in the insulating layer 215.

A connection portion 204 is provided in a region of the substrate 351 where the substrate 352 does not overlap. In the connection portion 204, the wiring 355 is electrically connected to the FPC 353 through a conductive layer 166 and a connection layer 242. An example is described in which the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

The light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### [Light-Emitting Apparatus 100H]

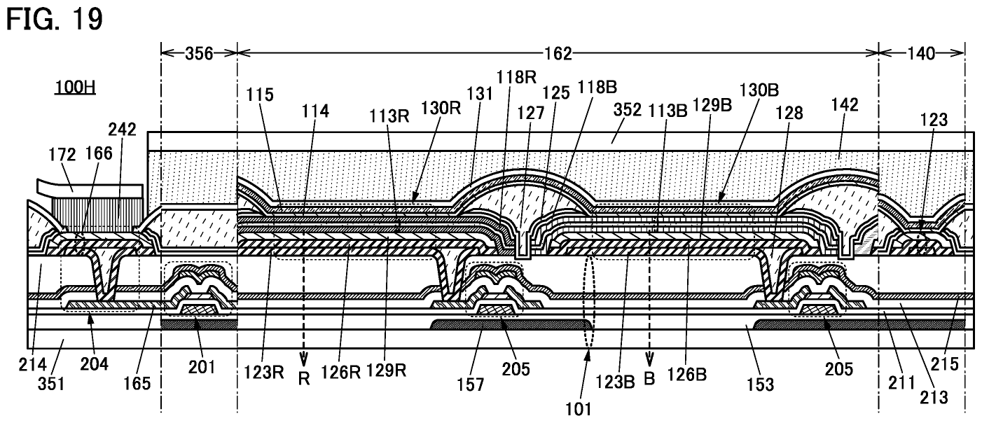

A light-emitting apparatus 100H illustrated in FIG. 19 differs from the light-emitting apparatus 100G illustrated in FIG. 18A mainly in having a bottom-emission structure.

Light from the light-emitting device is emitted toward the substrate 351. For the substrate 351, a material having a high visible-light-transmitting property is preferably used. By contrast, there is no limitation on the light-transmitting property of a material used for the substrate 352.

The light-blocking layer 157 is preferably formed between the substrate 351 and the transistor 201 and between the substrate 351 and the transistor 205. FIG. 19 illustrates an example in which the light-blocking layer 157 is provided over the substrate 351, an insulating layer 153 is provided over the light-blocking layer 157, and the transistors 201 and 205 and the like are provided over the insulating layer 153.

The light-emitting device 130R includes a conductive layer 123R, a conductive layer 126R over the conductive layer 123R, and a conductive layer 129R over the conductive layer 126R.

The light-emitting device 130B includes a conductive layer 123B, a conductive layer 126B over the conductive layer 123B, and a conductive layer 129B over the conductive layer 126B.

Materials having high visible-light-transmitting properties are used for the conductive layers 123R, 123B, 126R, 126B, 129R, and 129B. A material that reflects visible light is preferably used for the common electrode 155. As the conductive layer 123 provided in the connection portion 140, the conductive layer formed with the same material and in the same process as the conductive layers 123R, 126R, and 129R is preferably used.

Although not illustrated in FIG. 19, the light-emitting device 130G is also provided in the light-emitting apparatus 100H.

Although FIG. 19 and the like illustrate an example in which the top surface of the layer 128 includes a flat portion, the shape of the layer 128 is not particularly limited.

### [Light-Emitting Apparatus 100C]

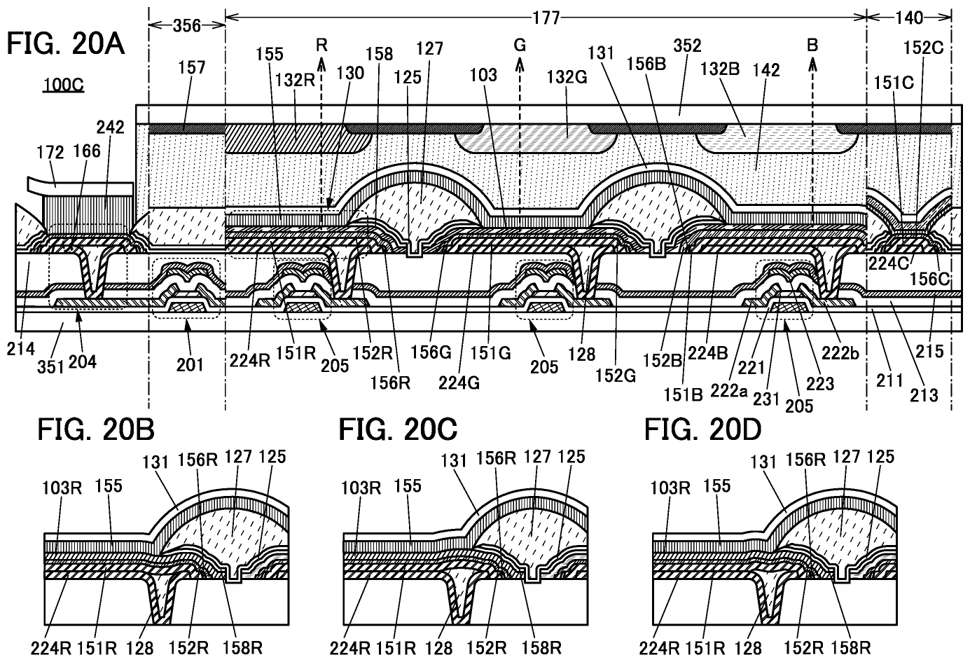

The light-emitting apparatus 100C illustrated in FIG. 20A is a variation example of the light-emitting apparatus 100B illustrated in FIG. 18A and differs from the light-emitting apparatus 100B mainly in including the coloring layers 132R, 132G, and 132B.

In the light-emitting apparatus 100C, the light-emitting device 130 includes a region overlapping with one of the coloring layers 132R, 132G, and 132B. The coloring layers 132R, 132G, and 132B can be provided on the surface of the substrate 352 on the substrate 351 side. Edge portions of the coloring layers 132R, 132G, and 132B can overlap with the light-blocking layer 157.

In the light-emitting apparatus 100C, the light-emitting device 130 can emit white light, for example. The coloring layer 132R, the coloring layer 132G, and the coloring layer 132B can transmit red light, green light, and blue light, respectively, for example. Note that in the light-emitting apparatus 100C, the coloring layers 132R, 132G, and 132B may be provided between the protective layer 131 and the adhesive layer 142.

Although the drawings like FIG. 18A and FIG. 20A illustrate an example in which the top surface of the layer 128 includes a flat portion, the shape of the layer 128 is not particularly limited. FIGS. 20B to 20D illustrate variation examples of the layer 128.

As illustrated in FIGS. 20B and 20D, the top surface of the layer 128 can have a shape in which its center and the vicinity thereof are depressed, i.e., a shape including a concave surface, in a cross-sectional view.

As illustrated in FIG. 20C, the top surface of the layer 128 can have a shape in which its center and the vicinity thereof bulge, i.e., a shape including a convex surface, in a cross-sectional view.

The top surface of the layer 128 may include one or both of a convex surface and a concave surface. The number of convex surfaces and the number of concave surfaces included in the top surface of the layer 128 are not limited and can each be one or two or more.

The level of the top surface of the layer 128 and the level of the top surface of the conductive layer 224R may be the same or substantially the same, or may be different from each other. For example, the level of the top surface of the layer 128 may be lower or higher than the level of the top surface of the conductive layer 224R.

In the example illustrated in FIG. 20B, it can be said that the layer 128 fits inside the depression portion of the conductive layer 224R. By contrast, as illustrated in FIG. 20D, the layer 128 is also present outside the depression portion of the conductive layer 224R, i.e., the top surface of the layer 128 may extend beyond the depression portion.

This embodiment can be combined as appropriate with the other embodiments or examples. In this specification, in the case where a plurality of structure examples are shown in one embodiment, the structure examples can be combined as appropriate.

### Embodiment 6

In this embodiment, electronic appliances of embodiments of the present invention will be described.

Electronic appliances of this embodiment include the light-emitting apparatus of one embodiment of the present invention in their display portions. The light-emitting apparatus of one embodiment of the present invention is highly reliable and can be easily increased in definition and resolution. Thus, the light-emitting apparatus of one embodiment of the present invention can be used for display portions of a variety of electronic appliances.

Examples of the electronic appliances include a digital camera, a digital video camera, a digital photo frame, a mobile phone, a portable game console, a portable information terminal, and an audio reproducing device, in addition to electronic appliances with a relatively large screen, such as a television device, desktop and notebook personal computers, a monitor of a computer and the like, digital signage, and a large game machine such as a pachinko machine.

In particular, the light-emitting apparatus of one embodiment of the present invention can have high definition, and thus can be favorably used for an electronic appliance having a relatively small display portion. Examples of such an electronic appliance include watch-type and bracelet-type information terminal devices (wearable devices) and wearable devices capable of being worn on a head, such as a VR device like a head-mounted display, a glasses-type AR device, and a mixed reality (MR) device.

The resolution of the light-emitting apparatus of one embodiment of the present invention is preferably as high as HD (number of pixels: 1280×720), FHD (number of pixels: 1920×1080), WQHD (number of pixels: 2560×1440), WQXGA (number of pixels: 2560×1600), 4K (number of pixels: 3840×2160), or 8K (number of pixels: 7680×4320). In particular, 4K resolution, 8K resolution, or higher resolution is preferable. The pixel density (definition) of the light-emitting apparatus of one embodiment of the present invention is preferably higher than or equal to 100 ppi, further preferably higher than or equal to 300 ppi, further preferably higher than or equal to 500 ppi, further preferably higher than or equal to 1000 ppi, still further preferably higher than or equal to 2000 ppi, still further preferably higher than or equal to 3000 ppi, still further preferably higher than or equal to 5000 ppi, yet further preferably higher than or equal to 7000 ppi. With such a light-emitting apparatus having one or both of high resolution and high definition, the electronic appliance can provide higher realistic sensation, sense of depth, and the like in personal use such as portable use or home use. There is no particular limitation on the screen ratio (aspect ratio) of the light-emitting apparatus of one embodiment of the present invention. For example, the light-emitting apparatus is compatible with a variety of screen ratios such as 1:1 (a square), 4:3, 16:9, and 16:10.

The electronic appliance in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic appliance in this embodiment can have a variety of functions. For example, the electronic appliance in this embodiment can have a function of displaying a variety of information (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

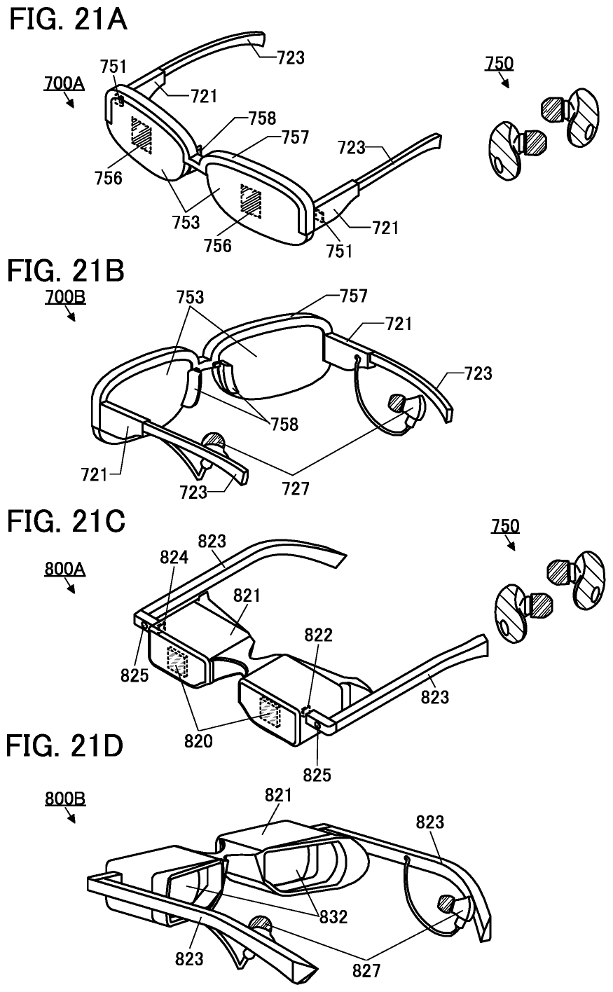

Examples of head-mounted wearable devices are described with reference to FIGS. 21A to 21D. These wearable devices have at least one of a function of displaying AR contents, a function of displaying VR contents, a function of displaying substitutional reality (SR) contents, and a function of displaying MR contents. The electronic appliance having a function of displaying contents of at least one of AR, VR, SR, MR, and the like enables the user to feel a higher level of immersion.

An electronic appliance 700A illustrated in FIG. 21A and an electronic appliance 700B illustrated in FIG. 21B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The light-emitting apparatus of one embodiment of the present invention can be used for the display panels 751. Thus, a highly reliable electronic appliance is obtained.

The electronic appliances 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic appliances 700A and 700B are electronic appliances capable of AR display.

In the electronic appliances 700A and 700B, a camera capable of capturing images of the front side may be provided as the image capturing portion. Furthermore, when the electronic appliances 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic appliances 700A and 700B are provided with a battery, so that they can be charged wirelessly and/or by wire.

A touch sensor module may be provided in the housing 721. The touch sensor module has a function of detecting a touch on the outer surface of the housing 721. Various types of processing can be executed by detecting a tap operation, a slide operation, or the like by the user with the touch sensor module. For example, a moving image can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

An electronic appliance 800A illustrated in FIG. 21C and an electronic appliance 800B illustrated in FIG. 21D each include a pair of display portions 820, a housing 821, a communication portion 822, a pair of wearing portions 823, a control portion 824, a pair of image capturing portions 825, and a pair of lenses 832.

The light-emitting apparatus of one embodiment of the present invention can be used in the display portions 820. Thus, a highly reliable electronic appliance is obtained.

The display portions 820 are positioned inside the housing 821 so as to be seen through the lenses 832. When the pair of display portions 820 display different images, three-dimensional display using parallax can be performed.

The electronic appliances 800A and 800B can function as electronic appliances for VR. The user who wears the electronic appliance 800A or the electronic appliance 800B can see images displayed on the display portions 820 through the lenses 832.

The electronic appliances 800A and 800B preferably include a mechanism for adjusting horizontally the positions of the lenses 832 and the display portions 820 so that the lenses 832 and the display portions 820 are positioned optimally in accordance with the positions of the user's eyes. Moreover, the electronic appliances 800A and 800B preferably include a mechanism for adjusting focus by changing the distance between the lenses 832 and the display portions 820.

The electronic appliance 800A or the electronic appliance 800B can be mounted on the user's head with the wearing portions 823. FIG. 21C, for instance, shows an example where the wearing portion 823 has a shape like a temple (also referred to as a joint or the like) of glasses; however, one embodiment of the present invention is not limited thereto. The wearing portion 823 can have any shape with which the user can wear the electronic appliance, for example, a shape of a helmet or a band.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of fields of view, such as a telescope field of view and a wide field of view.

Although an example where the image capturing portions 825 are provided is described here, a range sensor (hereinafter also referred to as a sensing portion) capable of measuring the distance between the user and an object may be provided. In other words, the image capturing portion 825 is one embodiment of the sensing portion. As the sensing portion, an image sensor or a range image sensor such as a light detection and ranging (LiDAR) sensor can be used, for example. By using images obtained by the camera and images obtained by the range image sensor, more information can be obtained and a gesture operation with higher accuracy is possible.

The electronic appliance 800A may include a vibration mechanism that functions as bone-conduction earphones. For example, at least one of the display portion 820, the housing 821, and the wearing portion 823 can include the vibration mechanism. Thus, without additionally requiring an audio device such as headphones, earphones, or a speaker, the user can enjoy video and sound only by wearing the electronic appliance 800A.

The electronic appliances 800A and 800B may each include an input terminal. To the input terminal, a cable for supplying a video signal from a video output device or the like, power for charging a battery provided in the electronic appliance, and the like can be connected.

The electronic appliance of one embodiment of the present invention may have a function of performing wireless communication with earphones 750. The earphones 750 include a communication portion (not illustrated) and has a wireless communication function. The earphones 750 can receive information (e.g., audio data) from the electronic appliance with the wireless communication function. For example, the electronic appliance 700A in FIG. 21A has a function of transmitting information to the earphones 750 with the wireless communication function. For another example, the electronic appliance 800A in FIG. 21C has a function of transmitting information to the earphones 750 with the wireless communication function.

The electronic appliance may include an earphone portion. The electronic appliance 700B in FIG. 21B includes earphone portions 727. For example, the earphone portion 727 can be connected to the control portion by a wiring. Part of the wiring that connects the earphone portion 727 and the control portion may be positioned inside the housing 721 or the wearing portion 723.

Similarly, the electronic appliance 800B in FIG. 21D includes earphone portions 827. For example, the earphone portion 827 can be connected to the control portion 824 by a wiring. Part of the wiring that connects the earphone portion 827 and the control portion 824 may be positioned inside the housing 821 or the wearing portion 823. Alternatively, the earphone portions 827 and the wearing portions 823 may include magnets. This is preferable because the earphone portions 827 can be fixed to the wearing portions 823 with magnetic force and thus can be easily housed.

The electronic appliance may include an audio output terminal to which earphones, headphones, or the like can be connected. The electronic appliance may include one or both of an audio input terminal and an audio input mechanism. As the audio input mechanism, a sound collecting device such as a microphone can be used, for example. The electronic appliance may have a function of a headset by including the audio input mechanism.

As described above, both the glasses-type device (e.g., the electronic appliances 700A and 700B) and the goggles-type device (e.g., the electronic appliances 800A and 800B) are preferable as the electronic appliance of one embodiment of the present invention.

The electronic appliance of one embodiment of the present invention can transmit information to earphones by wire or wirelessly.

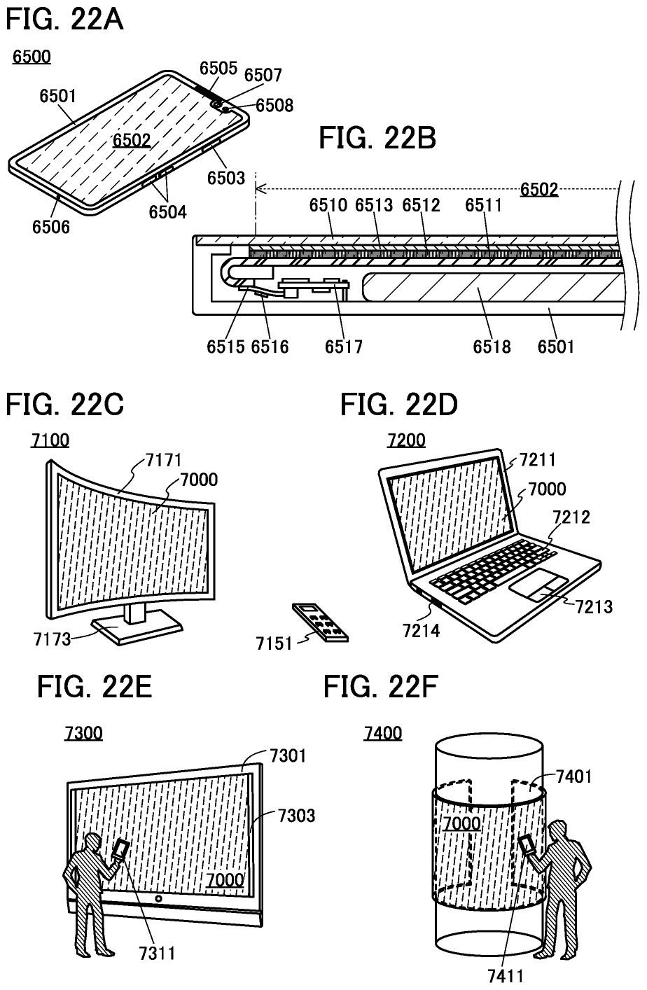

An electronic appliance 6500 illustrated in FIG. 22A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The light-emitting apparatus of one embodiment of the present invention can be used in the display portion 6502. Thus, a highly reliable electronic appliance is obtained.

FIG. 22B is a schematic cross-sectional view including an edge portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The light-emitting apparatus of one embodiment of the present invention can be used in the display panel 6511. Thus, an extremely lightweight electronic appliance can be obtained. Since the display panel 6511 is extremely thin, the battery 6518 with high capacity can be mounted without an increase in the thickness of the electronic appliance. An electronic appliance with a narrow bezel can be provided when part of the display panel 6511 is folded back and the portion connected to the FPC 6515 is provided on the back side of a pixel portion.

FIG. 22C illustrates an example of a television device. In a television device 7100, a display portion 7000 is incorporated in a housing 7171. Here, the housing 7171 is supported by a stand 7173.

The light-emitting apparatus of one embodiment of the present invention can be used in the display portion 7000. Thus, a highly reliable electronic appliance can be provided.

Operation of the television device 7100 illustrated in FIG. 22C can be performed with an operation switch provided in the housing 7171 and a separate remote controller 7151. Alternatively, the display portion 7000 may include a touch sensor, and the television device 7100 may be operated by touch on the display portion 7000 with a finger or the like. The remote controller 7151 may be provided with a display portion for displaying information output from the remote controller 7151. With operation keys or a touch panel of the remote controller 7151, channels and volume can be controlled and video displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 22D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The light-emitting apparatus of one embodiment of the present invention can be used in the display portion 7000. Thus, a highly reliable electronic appliance is obtained.

FIGS. 22E and 22F illustrate examples of digital signage.

Digital signage 7300 illustrated in FIG. 22E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 22F shows digital signage 7400 attached to a cylindrical pillar 7401. The digital signage 7400 includes the display portion 7000 provided along a curved surface of the pillar 7401.

In FIGS. 22E and 22F, the light-emitting apparatus of one embodiment of the present invention can be used in the display portion 7000. Thus, a highly reliable electronic appliance can be provided.

A larger area of the display portion 7000 can increase the amount of information that can be provided at a time. The display portion 7000 having a larger area attracts more attentions, so that the effectiveness of the advertisement can be increased, for example.

The touch panel is preferably used in the display portion 7000, in which case in addition to display of still or moving images on the display portion 7000, intuitive operation by a user is possible. Moreover, in the case of an application for providing information such as route information or traffic information, usability with intuitive operation can be enhanced

As illustrated in FIGS. 22E and 22F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

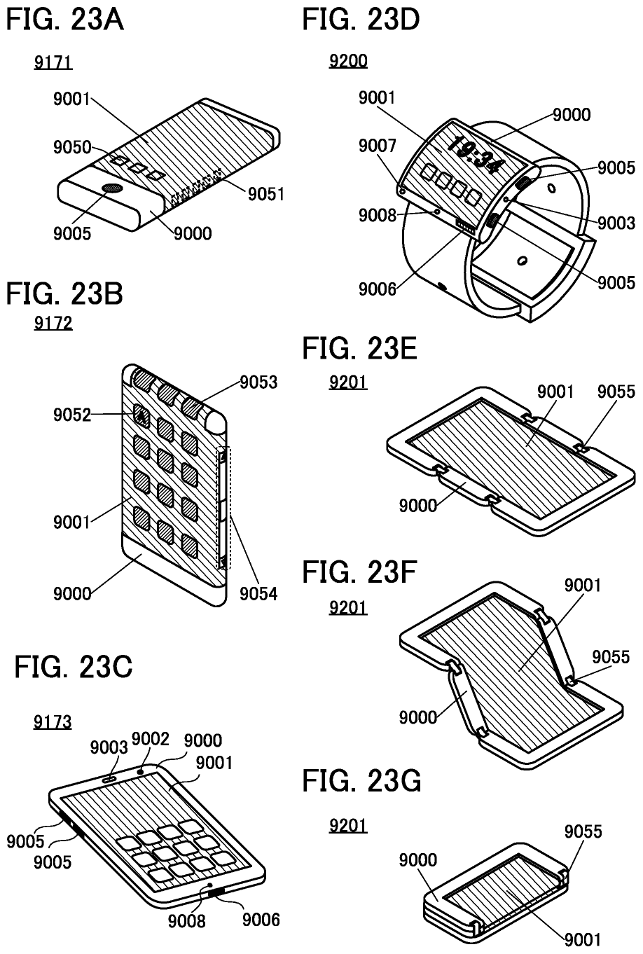

Electronic appliances illustrated in FIGS. 23A to 23G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic appliances illustrated in FIGS. 23A to 23G have a variety of functions. For example, the electronic appliances can have a function of displaying a variety of information (e.g., a still image, a moving image, and a text image) on the display portion, a touch panel function, a function of displaying a calendar, date, time, and the like, a function of controlling processing with the use of a variety of software (programs), a wireless communication function, and a function of reading out and processing a program or data stored in a recording medium. Note that the functions of the electronic appliances are not limited thereto, and the electronic appliances can have a variety of functions. The electronic appliances may include a plurality of display portions. The electronic appliances may be provided with a camera or the like and have a function of taking a still image or a moving image, a function of storing the taken image in a storage medium (an external storage medium or a storage medium incorporated in the camera), a function of displaying the taken image on the display portion, and the like.

The electronic appliances in FIGS. 23A to 23G are described in detail below.

FIG. 23A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 23A illustrates an example in which three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, the title and sender of an e-mail, an SNS message, or the like, the date, the time, remaining battery, and the radio field intensity. Alternatively, the icon 9050 or the like may be displayed at the position where the information 9051 is displayed.

FIG. 23B is a perspective view of a portable information terminal 9172. The portable information terminal 9172 has a function of displaying information on three or more surfaces of the display portion 9001. Here, an example in which information 9052, information 9053, and information 9054 are displayed on different surfaces is described. For example, the user of the portable information terminal 9172 can check the information 9053 displayed so as to be seen from above the portable information terminal 9172, with the portable information terminal 9172 put in a breast pocket of his/her clothes. Thus, the user can see the display without taking out the portable information terminal 9172 from the pocket and decide whether to answer the call, for example.

FIG. 23C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, the camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 23D is a perspective view of a watch-type portable information terminal 9200. The portable information terminal 9200 can be used as a Smartwatch (registered trademark), for example. The display surface of the display portion 9001 is curved, and an image can be displayed on the curved display surface. Furthermore, for example, mutual communication between the portable information terminal 9200 and a headset capable of wireless communication can be performed, and thus hands-free calling is possible. With the connection terminal 9006, the portable information terminal 9200 can perform mutual data transmission with another information terminal and charging. Note that the charging operation may be performed by wireless power feeding.

FIGS. 23E to 23G are perspective views of a foldable portable information terminal 9201. FIG. 23E is a perspective view showing the portable information terminal 9201 that is opened. FIG. 23G is a perspective view showing the portable information terminal 9201 that is folded. FIG. 23F is a perspective view showing the portable information terminal 9201 that is shifted from one of the states in FIGS. 23E and 23G to the other. The portable information terminal 9201 is highly portable when folded. When the portable information terminal 9201 is opened, a seamless large display region is highly browsable. The display portion 9001 of the portable information terminal 9201 is supported by three housings 9000 joined together by hinges 9055. The display portion 9001 can be folded with a radius of curvature greater than or equal to 0.1 mm and less than or equal to 150 mm, for example.

This embodiment can be combined as appropriate with any of the other embodiments or examples. In this specification, in the case where a plurality of structure examples are shown in one embodiment, the structure examples can be combined as appropriate.

### Example 1

### Synthesis Example 1

In this example, a synthesis method of 9′-(phenyl-d5)-9′H-9,3′:6′,9″-tercarbazole-1,1′,1″,2,2′,2″,3,3″,4,4′,4″,5,5′,5″,6,6″,7,7′,7″,8,8′,8″-d22 (abbreviation: PhCzGI-d27) represented by Structural Formula (100) in Embodiment 1 is described. The structure of PhCzGI-d27 is shown below.

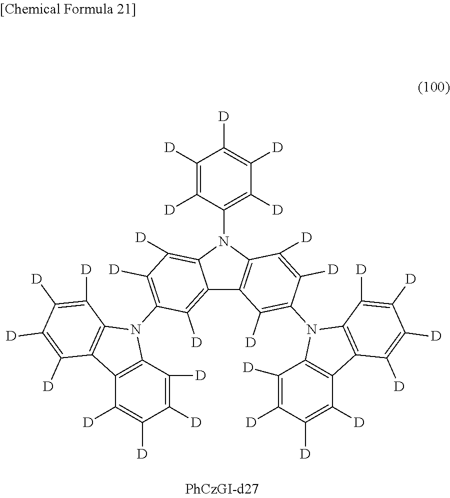 [SMILES: [2H]c1c([2H])c([2H])c(-n2c3c([2H])c([2H])c(-n4c5c([2H])c([2H])c([2H])c([2H])c5c5c([2H])c([2H])c([2H])c([2H])c54)c([2H])c3c3c([2H])c(-n4c5c([2H])c([2H])c([2H])c([2H])c5c5c([2H])c([2H])c([2H])c([2H])c54)c([2H])c([2H])c32)c([2H])c1[2H]]

### Step 1: Synthesis of 3,6-dibromo-9-(phenyl-d5)-9H-carbazole-1,2,4,5,7,8-d6

First, 11 g (26 mmol) of 3,6-dibromo-9-phenyl-9H-carbazole and 50 g of toluene-d8 were put in a 300 mL three-neck flask and stirred under a nitrogen stream. To this mixture solution was added 1.4 g (5.0 mmol) of molybdenum(V) chloride (abbreviation: MoCl₅), and the mixture was stirred at 80° C. for five hours. After the predetermined time elapsed, the resulting reaction mixture was subjected to extraction with toluene. The resulting residue was purified by silica gel column chromatography. As a developing solvent, a 5:1 hexane-toluene mixed solvent was used. The fraction was concentrated to give a solid. Ethanol was added to the obtained solid, and the solution was filtrated by suction, so that 6.5 g of a target white solid was obtained in 60% yield. The synthesis scheme of Step 1 is shown in (a-1) below.

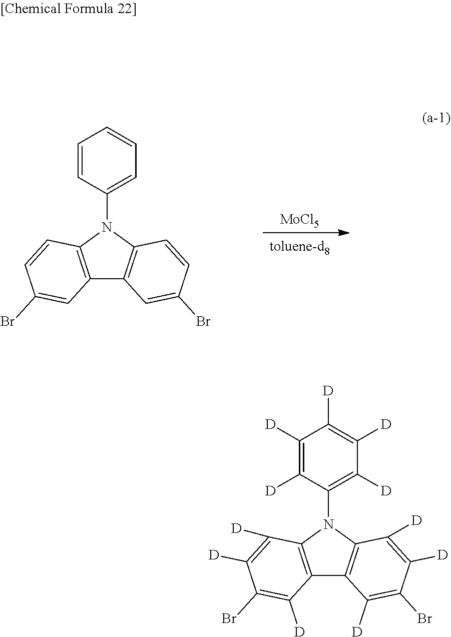 [SMILES: Brc1ccc2c(c1)c1cc(Br)ccc1n2-c1ccccc1 ; "MoCl5" ; [2H]c1c([2H])c([2H])c(-n2c3c([2H])c([2H])c(Br)c([2H])c3c3c([2H])c(Br)c([2H])c([2H])c32)c([2H])c1[2H]]

### Step 2: 9′-(phenyl-d5)-9′H-9,3′:6′,9″-tercarbazole-1,1′,1″,2,2′,2″,3,3″,4,4′,4″,5,5′,5″,6,6″,7,7′,7″,8,8′,8″-d22 (abbreviation: PhCzGI-d27)

Next, 4.1 g (9.9 mmol) of 3,6-dibromo-9-(phenyl-d5)-9H-carbazole-1,2,4,5,7,8-d6 synthesized in Step 1, 4.1 g (23 mmol) of carbazole-1,2,3,4,5,6,7,8-d8, 5.7 g (59 mmol) of sodium tert-butoxide (abbreviation: NaOtBu), 0.24 g (0.59 mmol) of 2-dicyclohexylphosphino-2′,6′-dimethoxy-biphenyl (abbreviation: Sphos), and 80 mL of xylene were put in a 200 mL three-neck flask, the air of the flask was replaced with nitrogen, and the mixture in the flask was stirred under reduced pressure to be degassed. After the degassing, 0.27 g (0.30 mmol) of tris(dibenzylideneacetone)palladium(0) was added, and the mixture was stirred at 140° C. under a nitrogen stream for 14 hours. After the predetermined time elapsed, the resulting reaction mixture was suction-filtered through a filter aid of Celite and alumina stacked in this order. The resulting filtrate was concentrated to give a solid. The solid was purified by silica column chromatography. As a developing solvent, a 5:1 hexane-toluene mixed solvent was used and then a 1:1 hexane-toluene mixed solvent was used. The fraction was concentrated to give a target solid. The solid was recrystallized from ethyl acetate to give 3.3 g of the solid in 55% yield. By a train sublimation method, 3.3 g of the solid was purified by train sublimation. The purification by sublimation was performed by heating at 285° C. under a pressure of 3.7 Pa with an argon flow rate of 15 mL/min for 24 hours. After the purification by sublimation, 2.2 g of the target white solid was obtained at a collection rate of 67%. The synthesis scheme of Step 2 is shown in (a-2) below.

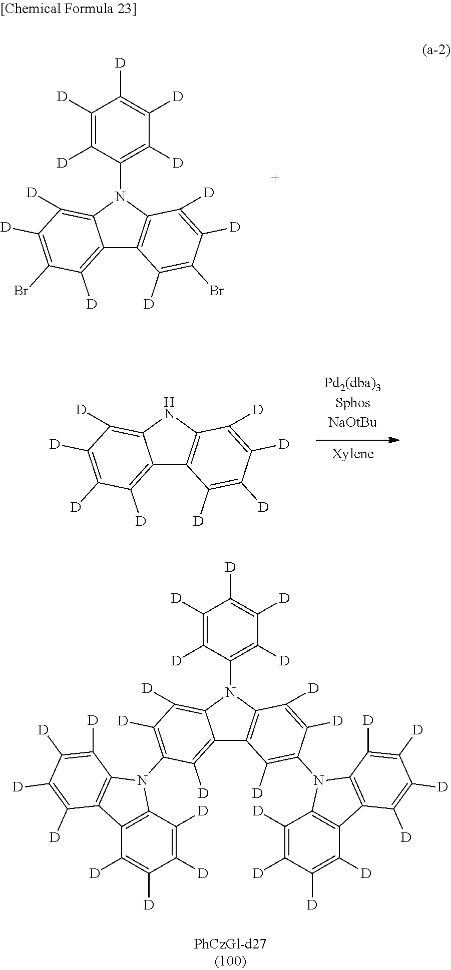 [SMILES: [2H]c1c([2H])c([2H])c(-n2c3c([2H])c([2H])c(Br)c([2H])c3c3c([2H])c(Br)c([2H])c([2H])c32)c([2H])c1[2H] ; [2H]c1c([2H])c([2H])c2c([nH]c3c([2H])c([2H])c([2H])c([2H])c32)c1[2H] ; [2H]c1c([2H])c([2H])c(-n2c3c([2H])c([2H])c(-n4c5c([2H])c([2H])c([2H])c([2H])c5c5c([2H])c([2H])c([2H])c([2H])c54)c([2H])c3c3c([2H])c(-n4c5c([2H])c([2H])c([2H])c([2H])c5c5c([2H])c([2H])c([2H])c([2H])c54)c([2H])c([2H])c32)c([2H])c1[2H]]

### <Measurement of Physical Property>

Next, an ultraviolet-visible absorption spectrum (hereinafter simply referred to as “absorption spectrum”) and an emission spectrum of PhCzGI-d27 in a solid thin film were measured. The solid thin film was deposited over a quartz substrate by a vacuum evaporation method.

The absorption spectrum was measured with a UV-visible spectrophotometer (U-4100, Hitachi High-Technologies Corporation). The absorption spectrum of the thin film was calculated using an absorbance (−log₁₀ [% T/(100−% R)]) obtained from the transmittance and reflectance of the thin film including the substrate. Note that % T represents transmittance and % R represents reflectance. The emission spectrum was measured with a fluorescence spectrophotometer (FP-8600, JASCO Corporation).

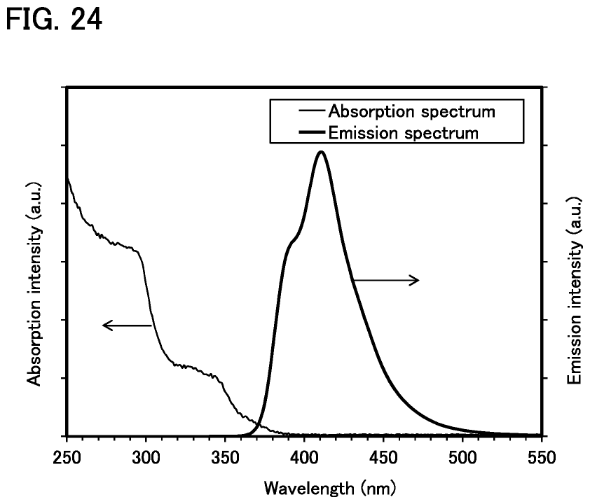

FIG. 24 shows the measurement results of the absorption and emission spectra of the deposited solid thin film. The horizontal axis represents wavelength, and the vertical axis represents absorption intensity and emission intensity.

As shown in FIG. 24, the solid thin film of PhCzGI-d27 exhibits absorption peaks at around 290 nm, 330 nm, and 358 nm, and emission wavelength peaks at around 390 nm and 411 nm (the excitation wavelength is 330 nm).

### Example 2

Light-emitting devices 2A to 2D of one embodiment of the present invention were fabricated and physical properties thereof were evaluated.

Structural formulas of organic compounds used for all of the light-emitting devices 2A to 2D are shown below.

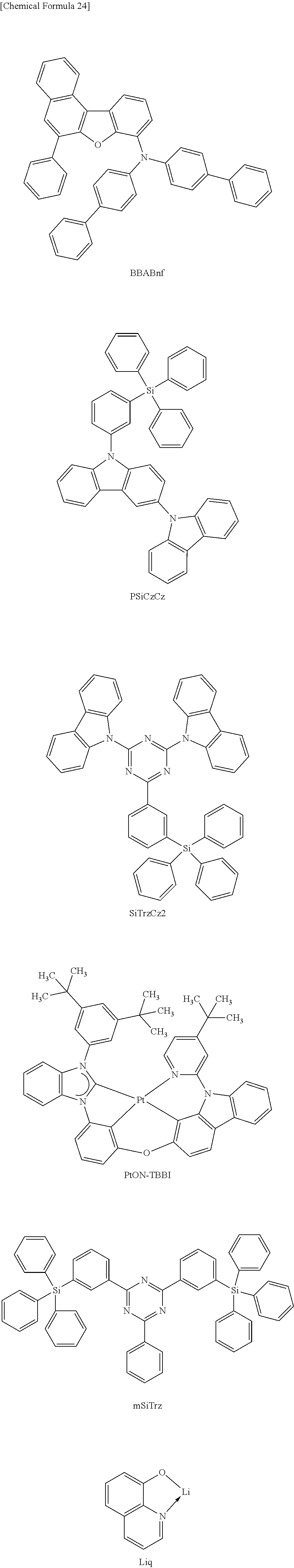

Structural formulas of organic compounds used independently in the light-emitting devices are shown below. Note that 9-[3-(triphenylsilyl)phenyl]-3,9′-(bi-9H-carbazole-d15) (abbreviation: PSiCzCz-d15) used in the light-emitting device 2B is an organic compound in which the carbazole skeleton of 9-[3-(triphenylsilyl)phenyl]-3,9′-bi-9H-carbazole (abbreviation: PSiCzCz) used in the light-emitting device 2A is deuterated. In addition, 9′-(phenyl-d5)-9′H-9,3′:6′,9″-tercarbazole-1,1′,1″,2,2′,2″,3,3″,4,4′,4″,5,5′,5″,6,6″,7,7′,7″,8,8′,8″-d22 (abbreviation:PhCzGI-d27) used in the light-emitting device 2D is an organic compound in which the carbazole skeleton of 9′-phenyl-9′H-9,3′:6′,9″-tercarbazole (abbreviation: PhCzGI) used in the light-emitting device 2C is deuterated.

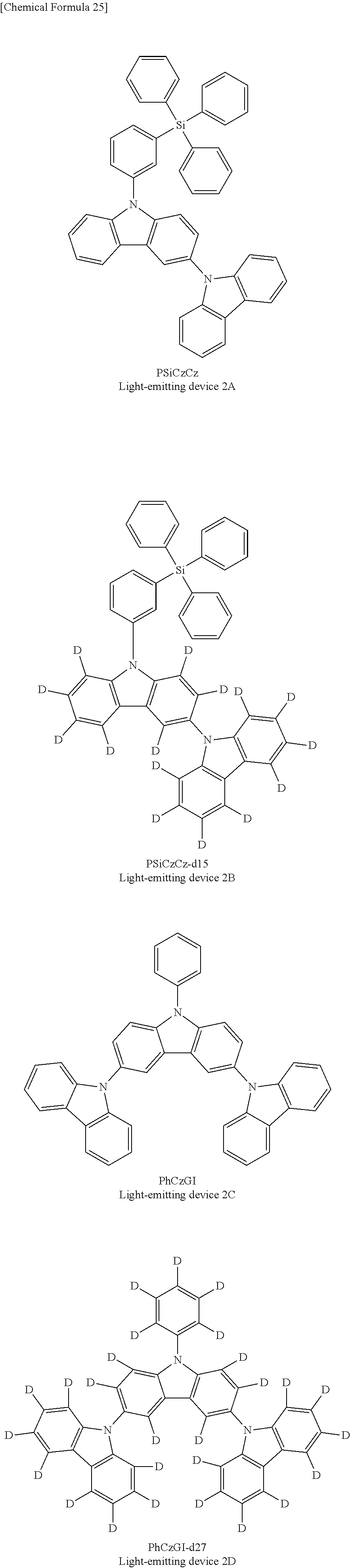

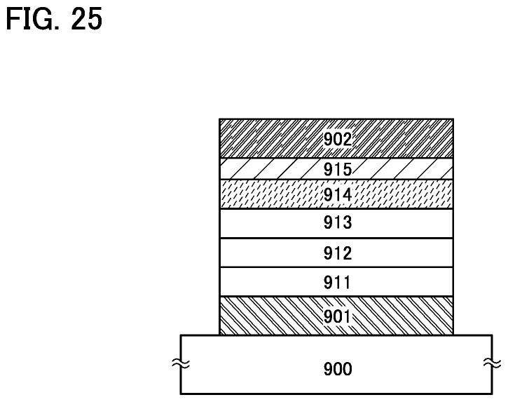

In each of the devices, as illustrated in FIG. 25, a hole-injection layer 911, a hole-transport layer 912, a light-emitting layer 913, an electron-transport layer 914, and an electron-injection layer 915 are stacked in this order over a first electrode 901 formed over a glass substrate 900, and a second electrode 902 is stacked over the electron-injection layer 915.

### <Fabrication Method of Light-Emitting Devices 2A to 2D>

As the first electrode 901 serving as a transparent electrode, indium tin oxide containing silicon oxide (ITSO) was deposited to a thickness of 70 nm over the glass substrate 900 by a sputtering method. The electrode area was set to 4 mm² (2 mm×2 mm).

Next, in pretreatment for forming the light-emitting device over the substrate, a surface of the substrate was washed with water and baking was performed at 200° C. for 1 hour. Then, the substrate was transferred into a vacuum evaporation apparatus where the pressure was reduced to approximately 1×10⁻⁴ Pa, and vacuum baking was performed at 180° C. for 60 minutes in a heating chamber of the vacuum evaporation apparatus, followed by natural cooling down to 30° C. or lower.

Then, the substrate provided with the first electrode 901 was fixed to a substrate holder provided in the vacuum evaporation apparatus such that the surface on which the first electrode 901 was formed faced downward. Over the first electrode 901, N,N-bis(4-biphenyl)-6-phenylbenzo[b]naphtho[1,2-d]furan-8-amine (abbreviation: BBABnf) and an electron acceptor material containing fluorine and having a molecular weight of 672 (OCHD-003) were deposited by co-evaporation to a thickness of 10 nm at the weight ratio of 1:0.1 (BBABnf:OCHD-003=1:0.1), whereby the hole-injection layer 911 was formed.

Next, BBABnf was deposited over the hole-injection layer 911 to a thickness of 30 nm, and then PSiCzCz was deposited to a thickness of 5 nm, whereby the hole-transport layer 912 was formed.

Subsequently, under the conditions shown in Table 1, the light-emitting layer 913 was formed over the hole-transport layer 912 to a thickness of 35 nm by co-evaporation using resistance heating of 9,9′-{6-[3-(triphenylsilyl)phenyl]-1,3,5-triazine-2,4-diyl}bis(9H-carbazole) (abbreviation: SiTrzCz2), a material X, and (2-{3-[3-(3,5-di-tert-butylphenyl)benzimidazol-1-yl-2-ylidene-κC²]phenoxy-κC²}-9-(4-tert-butyl-2-pyridinyl-κN)carbazole-2,1-diyl-κC)platinum(II) (abbreviation: PtON-TBBI) at the weight ratio of 0.435:0.435:0.13 (SiTrzCz2:X:PtON-TBBI=0.435:0.435:0.13). Note that X represents PSiCzCz, PSiCzCz-d15, PhCzGI, or PhCzGI-d27.

Specifically, for the light-emitting device 2A, SiTrzCz2, PSiCzCz, and PtON-TBBI were co-evaporated. For the light-emitting device 2B, SiTrzCz2, PSiCzCz-d15, and PtON-TBBI were co-evaporated. For the light-emitting device 2C, SiTrzCz2, PhCzGI, and PtON-TBBI were co-evaporated. For the light-emitting device 2D, SiTrzCz2, PhCzGI-d27, and PtON-TBBI were co-evaporated.

Then, the electron-transport layer 914 was formed over the light-emitting layer 913 in the following manner: 2-phenyl-4,6-bis[3-(triphenylsilyl)phenyl]-1,3,5-triazine (abbreviation: mSiTrz) was deposited to a thickness of 5 nm and then, mSiTrz and 8-quinolinolato-lithium (abbreviation: Liq) were co-deposited to a thickness of 20 nm at the weight ratio of 1:1 (mSiTrz:Liq=1:1).

Next, lithium fluoride (LiF) was deposited by evaporation to a thickness of 1 nm over the electron-transport layer 914, so that the electron-injection layer 915 was formed.

Then, aluminum (Al) was deposited to a thickness of 200 nm over the electron-injection layer 915 to form the second electrode.

The device structures of the light-emitting devices 2A to 2D are listed in Table 1. Note that X in Table 1 represents PSiCzCz, PSiCzCz-d15, PhCzGI or PhCzGI-d27.

TABLE 1

| | Materials | | | |
|---|---|---|---|---|
| Thickness | Light-emitting | Light-emitting | Light-emitting | Light-emitting |
| [nm] | device 2A | device 2B | device 2C | device 2D |

| | | |
|---|---|---|
| Second electrode | 200 | Al |
| Electron-injection | 1 | LiF |
| layer | | |
| Electron-transport | 20 | mSiTrz:Liq (1:1) |
| layer | 5 | mSiTrz |
| Light-emitting layer | 35 | SiTrzCz2:X:PtON-TBBI (0.435:0.435:0.13) |

| | | | |
|---|---|---|---|
| X = PSiCzCz | X = PSiCzCz-d15 | X = PhCzGI | X = PhCzGI-d27 |

| | | |
|---|---|---|
| Hole-transport | 5 | PSiCzCz |
| layer | 30 | BBABnf |
| Hole-injection layer | 10 | BBABnf:OCHD-003 (1:0.1) |
| First electrode | 70 | ITSO |

### <Characteristics of Light-Emitting Devices>

The light-emitting devices 2A to 2D were sealed using a glass substrate in a glove box containing a nitrogen atmosphere so as not to be exposed to the air (a sealing material was applied to surround the device and UV treatment and heat treatment at 80° C. for 1 hour were performed at the time of sealing). Then, the characteristics of the devices were measured.

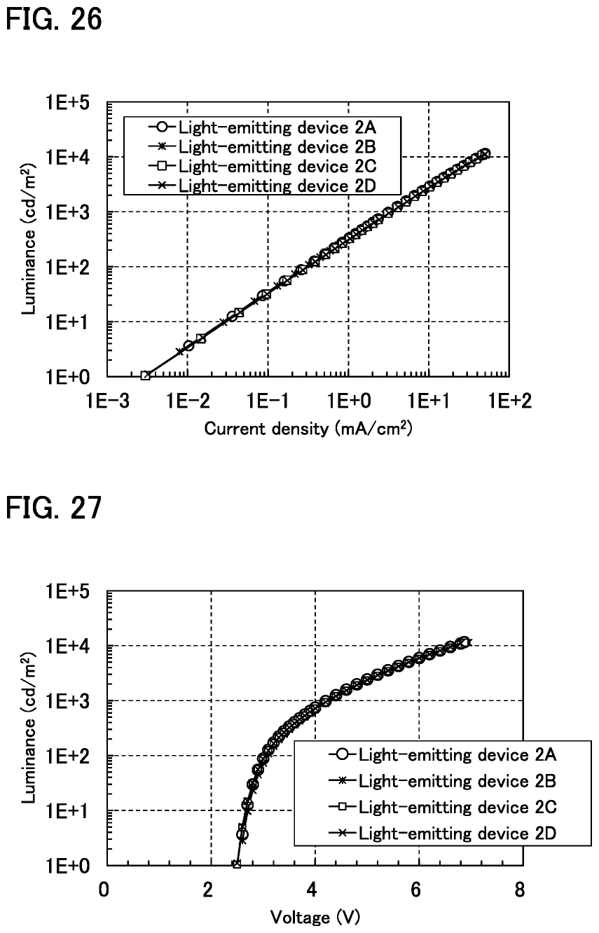

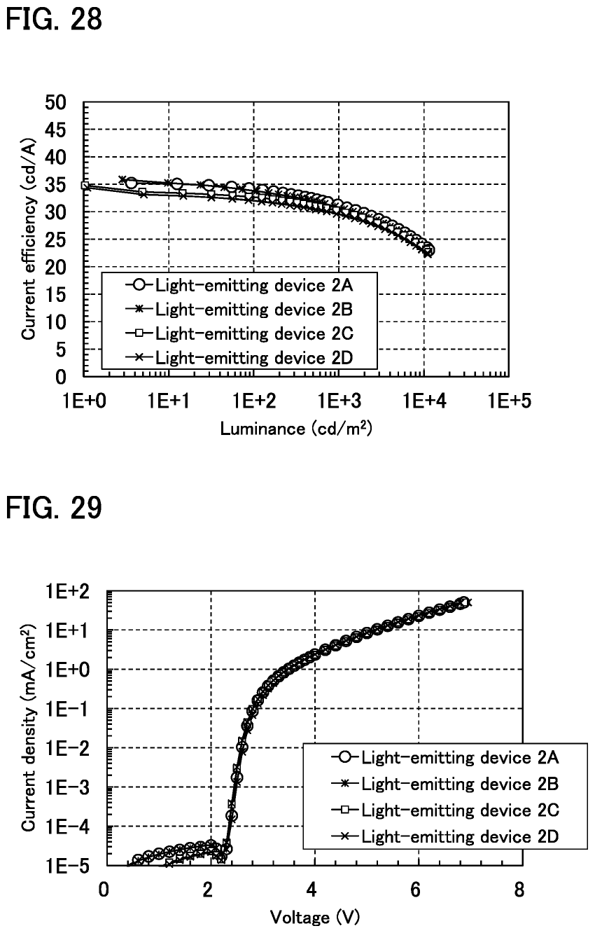

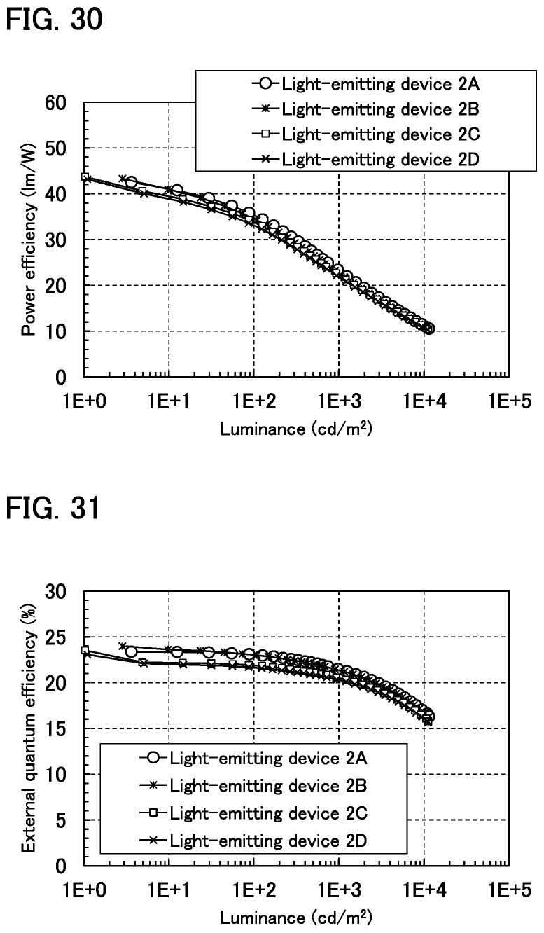

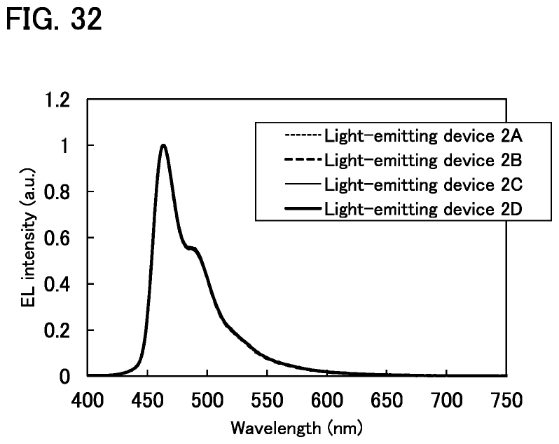

FIG. 26 shows the luminance-current density characteristics of the light-emitting devices 2A to 2D. FIG. 27 shows the luminance-voltage characteristics of the light-emitting devices 2A to 2D. FIG. 28 shows the current efficiency-luminance characteristics of the light-emitting devices 2A to 2D. FIG. 29 shows the current efficiency-voltage characteristics of the light-emitting devices 2A to 2D. FIG. 30 shows the power efficiency-luminance characteristics of the light-emitting devices 2A to 2D. FIG. 31 shows the external quantum efficiency-luminance characteristics of the light-emitting devices 2A to 2D. FIG. 32 shows the electroluminescence spectra of the light-emitting devices 2A to 2D.

The following table shows the main characteristics of the light-emitting devices at a luminance of 1000 cd/m². The luminance, CIE chromaticity, and emission spectra were measured with a spectroradiometer (SR-UL1R, TOPCON TECHNOHOUSE CORPORATION). The external quantum efficiency (EQE) was calculated from the luminance and the emission spectra measured with the spectroradiometer, on the assumption that the devices had Lambertian light-distribution characteristics.

TABLE 2

| | | Current | | | | Current | |
|---|---|---|---|---|---|---|---|
| | Voltage | density | Chromaticity | Chromaticity | Luminance | efficiency | EQE |
| | (V) | (mA/cm²) | x | y | (cd/m²) | (cd/A) | (%) |
| Light-emitting device 2A | 4.2 | 3.1 | 0.14 | 0.20 | 974 | 31 | 21 |
| Light-emitting device 2B | 4.2 | 2.9 | 0.14 | 0.20 | 900 | 31 | 21 |
| Light-emitting device 2C | 4.2 | 3.1 | 0.14 | 0.20 | 935 | 30 | 21 |
| Light-emitting device 2D | 4.2 | 3.2 | 0.14 | 0.20 | 942 | 30 | 20 |

As apparent from FIG. 26 to FIG. 32, the light-emitting devices 2A to 2D have equivalent device characteristics.

### <Reliability Test Result>

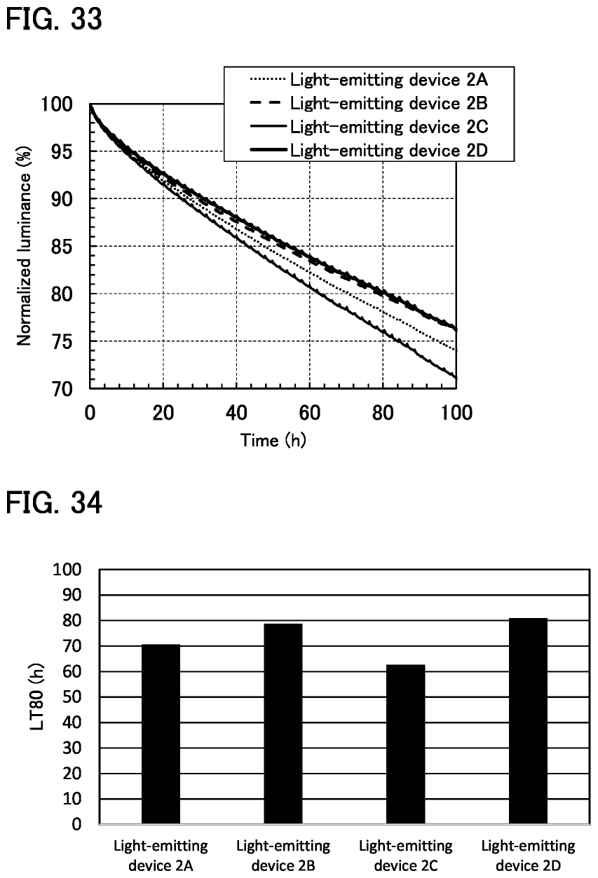

Furthermore, a reliability test was performed on the light-emitting devices 2A to 2D. FIG. 33 shows time-dependent changes in normalized luminance at the time of constant current density driving (10 [mA/cm²]). In FIG. 33, the vertical axis represents the luminance (%) normalized with the luminance at the time of the start of emission as 100%, and the horizontal axis represents the time (h). FIG. 34 shows LT80 (h), which is the time that has elapsed until the measured luminance decreases to 80% of the initial luminance, of the light-emitting devices 2A to 2D.

As shown in FIG. 32 and FIG. 34, LT80 (h) of the light-emitting device 2A was approximately 71 hours and LT80 (h) of the light-emitting device 2B was approximately 79 hour. That is, LT80 (h) of the light-emitting device 2B was 1.11 times longer than that of the light-emitting device 2A. The light-emitting device 2B has higher reliability. In addition, LT80 (h) of the light-emitting device 2C was approximately 63 hours and LT80 (h) of the light-emitting device 2D was approximately 81 hours. That is, LT80 (h) of the light-emitting device 2D was approximately 1.29 times longer than that of the light-emitting device 2C. The light-emitting device 2D has much higher reliability.

### <Measurement Result of PL Spectrum>

Here, photoluminescence (PL) spectra at room temperature and 10 K of mixed films of PSiCzCz, PSiCzCz-d15, PhCzGI, and PhCzGI-d27, which were used for the light-emitting device 2A to 2D respectively, and SiTrzCz2, which was used for all of the light-emitting device 2A to 2D, were measured.

Here, the PL spectrum at room temperature (for example, approximately, 290 K to 300 K (17° C. to 27° C.), preferably around 298 K (25° C.) was observed in the following manner: luminescence exhibited when photoexcited molecules return to a ground state from a singlet excited state was observed, which corresponds to a fluorescent spectrum. In addition, the photoluminescence (PL) spectrum at low temperature (a temperature in the temperature range of 4 K to 80 K, inclusive) was observed in the following manner: luminescence exhibited when photoexcited molecules return to a ground state from a triple excited state was observed, which correspond to a phosphorescent spectrum.

Note that the measurement of these spectra was performed on thin films with a thickness of 50 nm deposited on quartz substrates. The mixed film of SiTrzCz2 and PSiCzCz, PSiCzCz-d15, PhCzGI, or PhCzGI-d27 was deposited by co-evaporation of SiTrzCz2 and the organic compound (PSiCzCz, PSiCzCz-d15, PhCzGI, or PhCzGI-d27) at the weight ratio of 1:1 (SiTrzCz2:organic compound=1:1).

The fluorescent spectrum was measured with a fluorescence spectrophotometer, FP-8600DS (JASCO Corporation). The phosphorescent spectrum was measured with a microscope PL spectrometer, LabRAM HR-PL (HORIBA Scientific). In the measurement of the phosphorescent spectrum, irradiation with excitation light of 325 nm was performed for 0.1 sec and no detection or excitation was performed for 0.02 sec. Then, luminescence from the film at 10 K was detected for 0.86 sec and no detection or excitation was performed for 0.02 sec. These two steps were repeated to measure the phosphorescent spectrum.

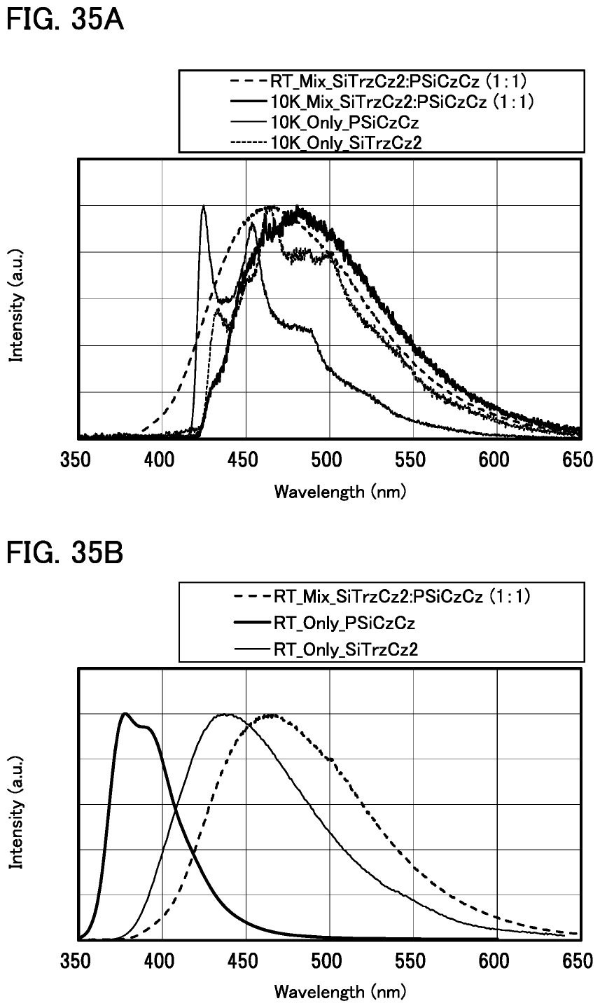

FIG. 35A shows the measurement result of the PL spectra at room temperature and 10 K of the mixed film of SiTrzCz2 and PSiCzCz, which is the organic compound used in the light-emitting device 2A. In addition to the result, PL spectra measured at 10 K of a film of only PSiCzCz and a film of only SiTrzCz2 were also shown as references. FIG. 35B shows the measurement result of the PL spectra at room temperature of the mixed film of SiTrzCz2 and PSiCzCz, which is the organic compound used in the light-emitting device 2A, the film of only PSiCzCz and the film of only SiTrzCz2. Note that explanatory notes are each shown in the order of the measurement temperature (room temperature or 10 K), the film state (a film of only a substance or a mixed film), and the organic compound in FIGS. 35A and 35B, FIGS. 36A and 36B, FIGS. 37A and 37B, FIGS. 38A and 38B, and FIG. 39.

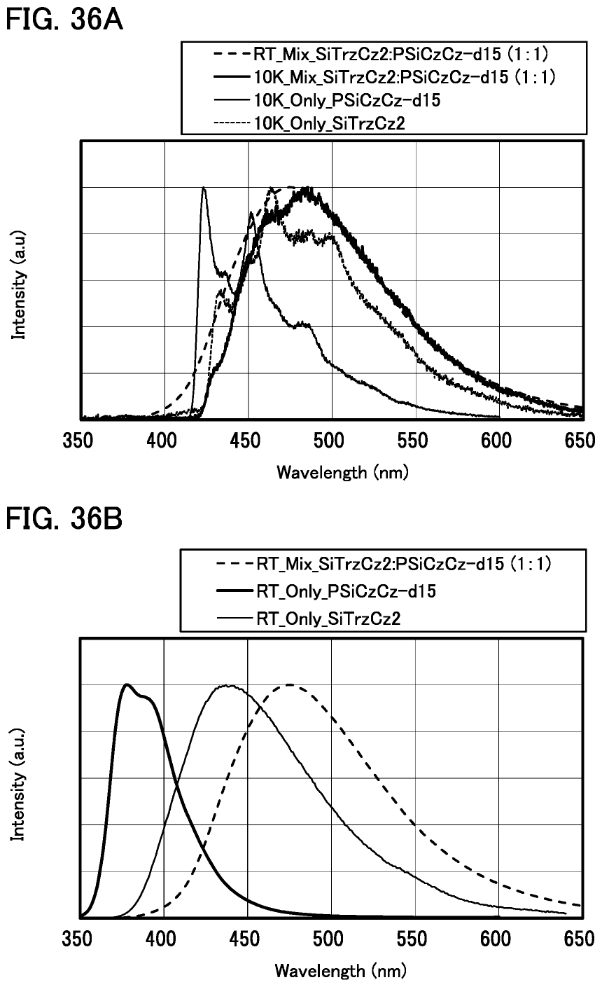

FIG. 36A shows the measurement result of the PL spectra at room temperature and 10 K of the mixed film of SiTrzCz2 and PSiCzCz-d15, which is the organic compound used in the light-emitting device 2B. In addition to the result, PL spectra measured at 10 K of a film of only PSiCzCz-d15 and the film of only SiTrzCz2 were also shown as references. FIG. 36B shows the measurement result of the PL spectra at room temperature of the mixed film of SiTrzCz2 and PSiCzCz-d15, which is the organic compound used in the light-emitting device 2B, the film of only PSiCzCz-d15 and the film of only SiTrzCz2.

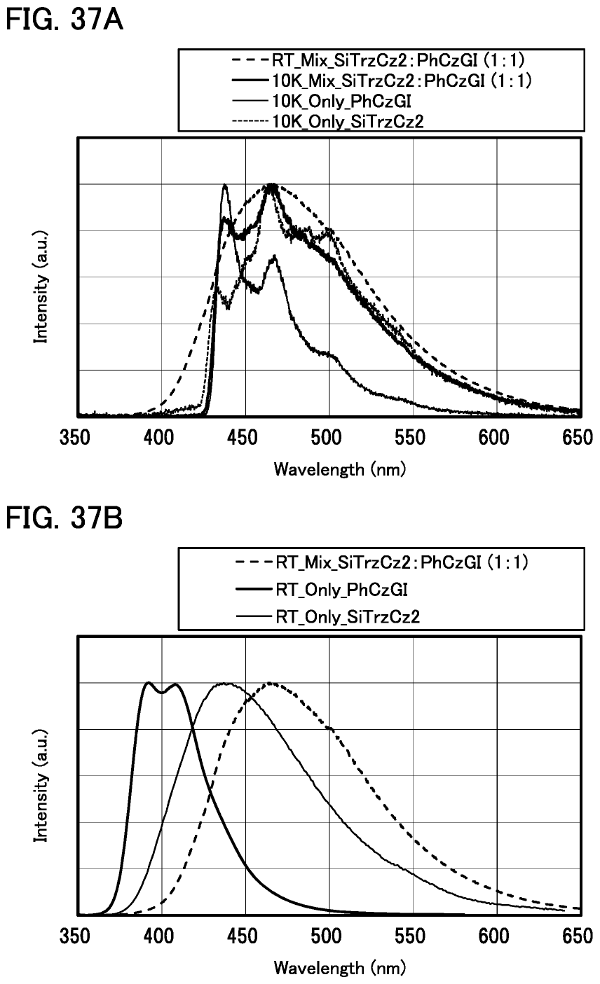

FIG. 37A shows the measurement result of the PL spectra at room temperature and 10 K of the mixed film of SiTrzCz2 and PhCzGI, which is the organic compound used in the light-emitting device 2C. In addition to the result, photoluminescence (PL) spectra measured at 10 K of a film of only PhCzGI and the film of only SiTrzCz2 were also shown as references. FIG. 37B shows the measurement result of the PL spectra at room temperature of the mixed film of SiTrzCz2 and PhCzGI, which is the organic compound used in the light-emitting device 2C, the film of only PhCzGI and the film of only SiTrzCz2.

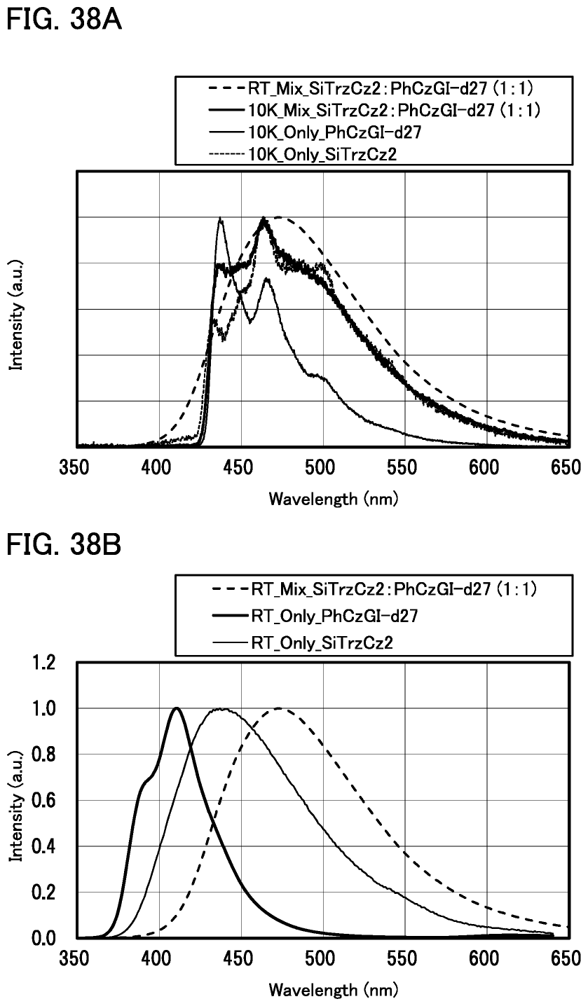

FIG. 38A shows the measurement result of the PL spectra measured at room temperature and 10 K of the mixed film of SiTrzCz2 and PhCzGI-d27, which is an organic compound used in the light-emitting device 2D. In addition to the result, PL spectra measured at 10 K of a film of only PhCzGI-d27 and the film of only SiTrzCz2 were also shown as references. FIG. 38B shows the measurement result of the phosphorescence (PL) spectra at room temperature of the mixed film of SiTrzCz2 and PhCzGI-d27 of the organic compound used in the light-emitting device 2D, the film of only PhCzGI-d27 and the film of only SiTrzCz2.

As shown in FIG. 35B, the emission spectrum of the mixed film of SiTrzCz2 and PSiCzCz is different from the spectrum obtained by superimposing the emission spectrum of the film of only PSiCzCz and the emission spectrum of the film of only SiTrzCz2 and is shifted to a longer wavelength than the emission spectra of the film of only PSiCzCz and the film of only SiTrzCz2. Thus, it is found that SiTrzCz2 and PSiCzCz in the mixed film were excited at room temperature to form an exciplex, and a fluorescent component of the emission spectrum originating from the exciplex was observed.

As shown in FIG. 36B, it is found that SiTrzCz2 and PSiCzCz-d15 in the mixed film were also excited at room temperature to form an exciplex, and a fluorescent component of the emission spectrum originating from the exciplex was observed. Furthermore, as shown in FIG. 36A, in the mixed films each, the spectrum measured at 10 K is shifted to a longer wavelength than the spectrum measured at room temperature; thus, it is inferred that phosphorescent component of the emission spectrum originated from the exciplex was observed.

As shown in FIG. 35A, the emission edge (418 nm, 2.967 eV) on the short wavelength side of the emission spectrum (phosphorescent spectrum) of PSiCzCz is located on a shorter wavelength than the emission edge (422 nm, 2.938 eV) on the short wavelength side of the emission spectrum (phosphorescent spectrum) at 10 K of the mixed film of PSiCzCz and SiTrzCz2.

The energy on the short wavelength side of the phosphorescent spectrum can be regarded as the energy of the lowest triplet excited level (T₁ level) of the organic compound. Moreover, the phosphorescent spectrum of the mixed film of PSiCzCz and SiTrzCz2 has a spectrum shape whose vibration structure is not so clear, and the tail of the spectrum on the long wavelength side is very similar to the tail of the fluorescent spectrum at room temperature of the exciplex; therefore, it is considered that the phosphorescent spectrum of the mixed film nearly reflects phosphorescence exhibited from the exciplex. In other words, the energy on the short wavelength side of the phosphorescent spectrum of the mixed film of PSiCzCz and SiTrzCz2 can be regarded as the T₁ level of the exciplex. This shows that in the light-emitting device 2A, the T₁ level (418 nm, 2.967 eV) of PSiCzCz is higher than the T₁ level (422 nm, 2.938 eV) of the exciplex. Accordingly, it is considered that light emission in the light-emitting device 2A is obtained as the result of energy transfer from the T₁ level of the exciplex formed by PSiCzCz and SiTrzCz2 to the T₁ level of the light-emitting substance.

As shown in FIG. 36A, the emission edge (417 nm, 2.974 eV) on the short wavelength side of the emission spectrum (phosphorescent spectrum) of PSiCzCz-d15 is located at a shorter wavelength than the emission edge (422 nm, 2.938 eV) on the short wavelength side of the emission spectrum (phosphorescent spectrum) at 10 K of the mixed film of PSiCzCz-d15 and SiTrzCz2.

The energy on the short wavelength side of the phosphorescent spectrum can be regarded as the energy of the lowest triplet excited level (T₁ level) of the organic compound. Moreover, the phosphorescent spectrum of the mixed film of PSiCzCz-d15 and SiTrzCz2 has a spectrum shape whose vibration structure is not so clear, and the tail of the spectrum on the long wavelength side is very similar to the tail of the fluorescent spectrum at room temperature of the exciplex; therefore, it is considered that the phosphorescent spectrum of the mixed film nearly reflects phosphorescence exhibited from the exciplex. In other words, the energy on the short wavelength side of the phosphorescence spectrum of the mixed film of PSiCzCz-d15 and SiTrzCz2 can be regarded as the T₁ level of the exciplex. This shows that in the light-emitting device 2B, the T₁ level of PSiCzCz-d15 (417 nm, 2.974 eV) is higher than the T₁ level (422 nm, 2.938 eV) of the exciplex by 0.036 eV. Accordingly, it is considered that light emission in the light-emitting device 2B is obtained as the result of energy transfer from the T₁ level of the exciplex formed by PSiCzCz-d15 and SiTrzCz2 to the T₁ level of the light-emitting substance.

As shown in FIG. 37B, the emission spectrum of the mixed film of SiTrzCz2 and PhCzGI is different from the spectrum obtained by superimposing the spectrum of the film of only PhCzGI and the spectrum of the film of only SiTrzCz2 and is shifted to a longer wavelength than the emission spectra of the film of only PhCzGI and the film of only SiTrzCz2. Thus, it is found that SiTrzCz2 and PhCzGI in the mixed film was excited at room temperature to form an exciplex, and a fluorescent component of the emission spectrum originating from the exciplex was observed. On the other hand, in FIG. 37A, in the case of 10 K, a spectrum in which the phosphorescent spectrum of the film of only PhCzGI and the phosphorescent spectrum of the film of only SiTrzCz2 were superimposed was observed.

In addition, as in the mixed film of PhCzGI and SiTrzCz2, as shown in FIG. 38A and FIG. 38B, an exciplex was formed by excitation at room temperature in the mixed film of PhCzGI-d27 and SiTrzCz2, and a fluorescent component of the emission spectrum originating from the exciplex was observed. On the other hand, in the case of 10 K, a plurality of peaks (local maximum values) were observed, and a spectrum in which the phosphorescent spectrum of PhCzGI-d27 and the phosphorescent spectrum of SiTrzCz2 were superimposed was observed.

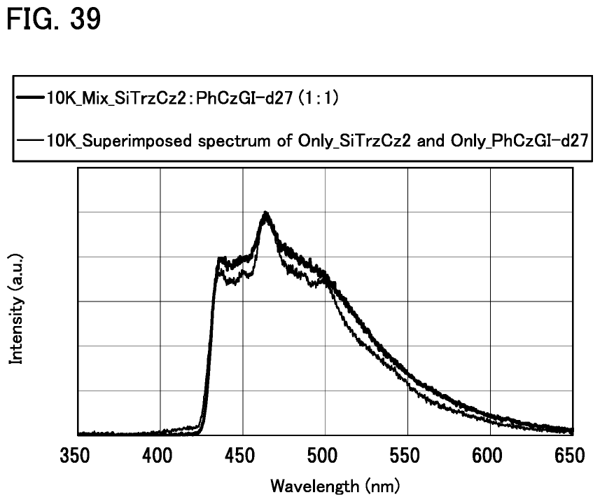

FIG. 39 shows measurement results of a PL spectrum at 10 K of the mixed film of SiTrzCz2 and PhCzGI-d27, which is an organic compound used in the light-emitting device 2D. In addition, a spectrum in which PL spectra measured at 10 K of the film of only PhCzGI-d27 and the film of only SiTrzCz2 were superimposed was also shown as a reference. As shown in FIG. 39, the PL spectrum measured at 10 K of the mixed film of PhCzGI-d27 and SiTrzCz2 is inferred to be a spectrum in which the PL spectra of the film of only PhCzGI-d27 and the film of only SiTrzCz2 are superimposed.

As described above, as shown in FIG. 37A, in the emission spectrum (phosphorescent spectrum) at 10 K of the mixed film of PhCzGI and SiTrzCz2, a superimposed spectrum of the phosphorescent spectrum observed in the measurement of the film of only PhCzGI and the phosphorescent spectrum observed in the measurement of the film of only SiTrzCz2 was observed.

In other words, in the light-emitting device 2C, the T₁ levels of PhCzGI and SiTrzCz2 are lower than the T₁ level of the exciplex of the mixed film of PhCzGI and SiTrzCz2. Accordingly, in the light-emitting device 2C, light emission is basically generated by energy transfer to the T₁ level of the light-emitting substance from the T₁ level of PhCzGI or SiTrzCz2, not from the T₁ level of the exciplex formed by PhCzGI and SiTrzCz2. In particular, the T₁ level (429 nm, 2.890 eV) of PhCzGI is slightly lower than the T₁ level (420 nm, 2.952 eV) of SiTrzCz2; therefore, the energy transfer from the T₁ level of PhCzGI is probably dominant. In other words, under electrical excitation, an exciplex is likely to be formed by PhCzGI and SiTrzCz2 due to carrier recombination, and the T₁ level energy of the exciplex formed is inferred to be transferred to the T₁ level of the light-emitting substance through the T₁ level of PhCzGI before deactivation and be emitted as light.

As described above, as shown in FIG. 38A, the emission spectrum (phosphorescent spectrum) at 10 K of the mixed film of PhCzGI-d27 and SiTrzCz2 is observed to be a superimposed spectrum of the phosphorescent spectrum observed in the measurement of the film of only PhCzGI-d27 and the phosphorescent spectrum observed in the measurement of the film of only SiTrzCz2.

In other words, in the light-emitting device 2D, the T₁ levels of PhCzGI-d27 and SiTrzCz2 are lower than the T₁ level of the exciplex of the mixed film of PhCzGI-d27 and SiTrzCz2. Accordingly, in the light-emitting device 2D, light emission is basically generated by energy transfer to the T₁ level of the light-emitting substance from the T₁ level of PhCzGI-d27 or SiTrzCz2, not from the T₁ level of the exciplex of PhCzGI-d27 and SiTrzCz2. In particular, the T₁ level (428 nm, 2.897 eV) of PhCzGI-d27 is slightly lower than the T₁ level (420 nm, 2.952 eV) of SiTrzCz2; therefore, the energy transfer from the T₁ level of PhCzGI-d27 occurs easily. In other words, under electrical excitation, an exciplex is likely to be formed by PhCzGI-d27 and SiTrzCz2 due to carrier recombination, and the T₁ level energy of the exciplex formed is inferred to be transferred to the T₁ level of the light-emitting substance through the T₁ level of PhCzGI-d27 before deactivation and be emitted as light.

As shown in FIG. 38A and FIG. 39, when the T₁ levels of the plurality of organic compounds used as the host materials are close to each other, the phosphorescent spectrum of the mixed film of the organic compounds is observed to be a spectrum in which the phosphorescent spectra of the organic compounds are superimposed. This is because the T₁ levels of the organic compounds are close to each other. It is a preferred mode in which a plurality of energy transfer paths are generated.

The difference between the T₁ levels of PhCzGI-d27 and SiTrzCz2 in FIG. 38A is 0.055 eV, as calculated by the formula: 2.952-2.897=0.055 eV. The difference between the T₁ levels of PhCzGI and SiTrzCz2 in FIG. 37A is 0.062 eV, as calculated by the formula: 2.952-2.890=0.062 eV. Accordingly, the difference between the T₁ levels of the plurality of organic compounds (the first organic compound and the second organic compound of the present invention) used as the host materials is preferably 0.100 eV or less, further preferably 0.090 eV or less, still further preferably 0.080 eV or less, yet still further preferably 0.070 eV or less.

As a result, as shown in FIGS. 37A and 37B and FIGS. 38A and 38B, in each of the light-emitting devices 2C and 2D, the energy transfer from the triplet excited state of PhCzGI (the light-emitting device 2C) and PhCzGI-d27 (the light-emitting device 2D) to the triplet excited state of the light-emitting substance are dominant. Since the molecular structure of PhCzGI is unstable, the carbazole skeleton of PhCzGI is deuterated to stabilize the molecular structure in the light-emitting device 2D by reducing dissociation of a carbon-hydrogen bond or nonradiative deactivation due to vibration. In other words, PhCzGI-d27, which is deuterated PhCzGI, contains deuterium, and thus is an organic compound having a stable excited state and a stable molecular structure.

Therefore, the light-emitting device 2D including PhCzGI-d27 is considered to have an improved reliability approximately 1.29 times higher than that of the light-emitting device 2C including PhCzGI. In the light-emitting device 2D, the path for light emission includes a second path of energy transfer from the T₁ level of the exciplex to the T₁ level of the light-emitting substance through the T₁ level of PhCzGI-d27, in addition to a first path of energy transfer directly from the T₁ level of the exciplex to the T₁ level of the light-emitting substance, whereby the reliability of light-emitting device 2D including PhCzGI-d27 is higher than that of the light-emitting device 2B including PSiCzCz-d15, which is deuterated PSiCzCz.

The above shows that a high-reliable light-emitting device can be provided by using one embodiment of the present invention.

### Example 3

### Synthesis Example 2

This example describes a synthesis method of 9′-[3-(triphenylsilyl)phenyl]-9′H-9,3′:6′,9″-tercarbazole-1,1′,1″,2,2′,2″,3,3″,4,4′,4″,5,5′,5″,6,6″,7,7′,7″,8,8′,8″-d22 (abbreviation: PSiCzGI-d22) represented by Structural Formula (101) described in Embodiment 1. The structure of PSiCzGI-d22 is shown below.

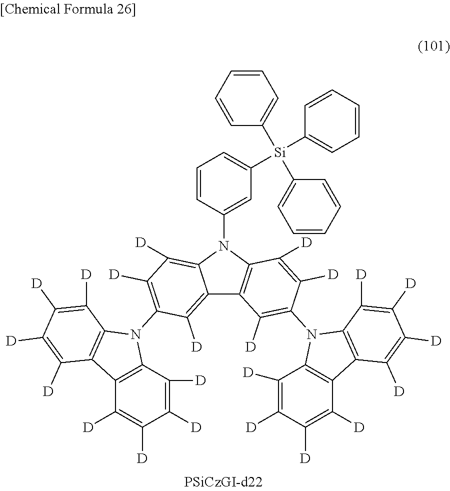 [SMILES: [2H]c1c([2H])c([2H])c2c(c1[2H])c1c([2H])c([2H])c([2H])c([2H])c1n2-c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1cccc([Si](c2ccccc2)(c2ccccc2)c2ccccc2)c1]

### Step 1: Synthesis of 9-[3-(triphenylsilyl)phenyl]-9H-carbazole-1,2,3,4,5,6,7,8-d8

First, 4.1 g (24 mmol) of carbazole-1,2,3,4,5,6,7,8-d8, 12 g (28 mmol) of (3-bromophenyl)triphenylsilane, 6.8 g (71 mmol) of sodium tert-butoxide (abbreviation: NaOtBu), 0.58 g (1.4 mmol) of 2-dicyclohexylphosphino-2′,6′-dimethoxybiphenyl (abbreviation: Sphos), and 120 mL of xylene were put in a 200 mL three-neck flask, the air of the flask was replaced with nitrogen, and the mixture in the flask was stirred under a reduced pressure in the flask for degassing of the mixture. After the degassing, 0.65 g (0.71 mmol) of tris(dibenzylideneacetone)dipalladium(0) was added thereto, and the mixture was stirred at 140° C. under a nitrogen stream for 7 hours. After the predetermined time elapsed, the reaction mixture was suction-filtered through a filter aid of Celite and alumina stacked in this order. The resulting filtrate was concentrated to give a solid. The obtained solid was purified by silica column chromatography. As a developing solvent, a 3:1 hexane-toluene mixed solvent was used, and the proportion of toluene was increased gradually and only toluene was used finally. The obtained fraction was concentrated so that 3.5 g of a target solid was obtained in 31% yield. The synthesis scheme of Step 1 is shown in (b-1) below.

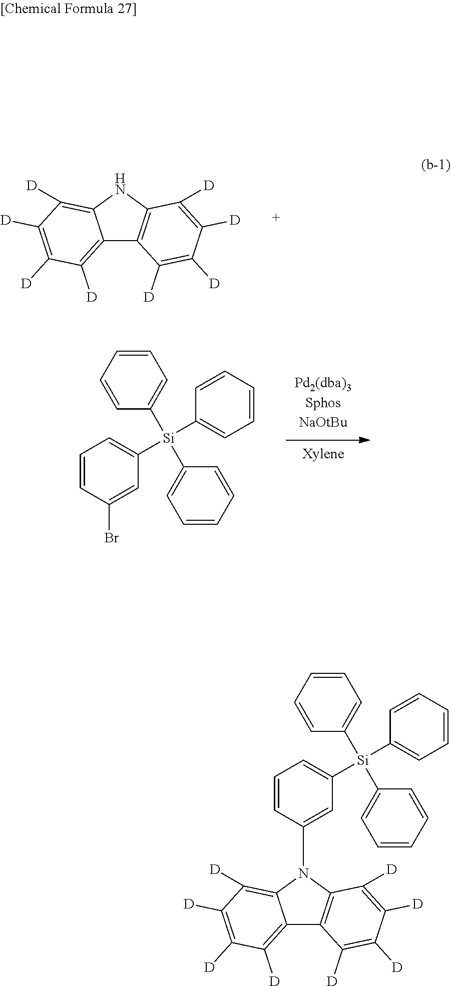 [SMILES: [2H]c1c([2H])c([2H])c2c([nH]c3c([2H])c([2H])c([2H])c([2H])c32)c1[2H] ; Brc1cccc([Si](c2ccccc2)(c2ccccc2)c2ccccc2)c1 ; [2H]c1c([2H])c([2H])c2c(c1[2H])c1c([2H])c([2H])c([2H])c([2H])c1n2-c1cccc([Si](c2ccccc2)(c2ccccc2)c2ccccc2)c1]

### Step 2: Synthesis of 3,6-dibromo-9-[3-(triphenylsilyl)phenyl]-9H-carbazole-1,2,4,5,7,8-d6

Then, 3.5 g (6.8 mmol) of 9-[3-(triphenylsilyl)phenyl]-9H-carbazole-1,2,3,4,5,6,7,8-d8 synthesized in Step 1 and 68 mL of ethyl acetate were put in a 500 mL three-neck flask and the air of the flask was replaced with nitrogen and the mixture in the flask was stirred under a reduced pressure in the flask for degassing of the mixture. After the degassing, 2.4 g (14 mmol) of N-bromosuccinimide was added, and the mixture was stirred at room temperature for 22 hours, and then stirred by heating at 80° C. for 7 hours. After the predetermined time elapsed, the reaction mixture was subjected to extraction by ethyl acetate, and then the resulting residue was purified by silica gel column chromatography. As a developing solvent, a 4:1 hexane-toluene mixed solvent was used. The obtained fraction was concentrated to give a solid. The obtained solid was recrystallized with the use of a mixed solvent of hexane and ethyl acetate, so that 3.5 g (5.2 mmol) of a target white solid was obtained in 77% yield. A synthesis scheme of Step 2 is shown in (b-2) below.

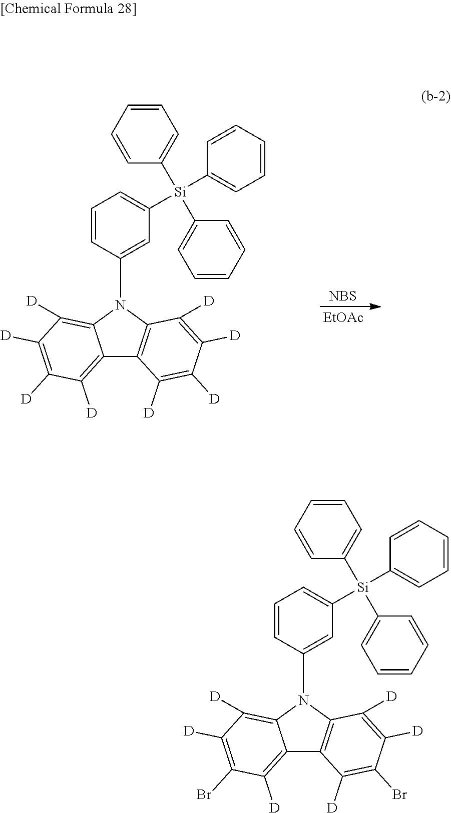 [SMILES: "EtOAc" ; [2H]c1c([2H])c([2H])c2c(c1[2H])c1c([2H])c([2H])c([2H])c([2H])c1n2-c1cccc([Si](c2ccccc2)(c2ccccc2)c2ccccc2)c1 ; "NBS" ; [2H]c1c(Br)c([2H])c2c3c([2H])c(Br)c([2H])c([2H])c3n(-c3cccc([Si](c4ccccc4)(c4ccccc4)c4ccccc4)c3)c2c1[2H]]

### Step 3: Synthesis of 9′-[3-(triphenylsilyl)phenyl]-9′H-9,3′:6′,9″-tercarbazole-1,1′,1″,2,2′,2″,3,3″,4,4′,4″,5,5′,5″,6,6″,7,7′,7″,8,8′,8″-d22 (abbreviation: PSiCzGI-d22)

Then, 3.4 g (5.1 mmol) of 3,6-dibromo-9-[3-(triphenylsilyl)phenyl]-9H-carbazole-1,2,4,5,7,8-d6 synthesized in Step 2, 2.1 g (12 mmol) of carbazole-1,2,3,4,5,6,7,8-d8, 2.9 g (31 mmol) of sodium tert-butoxide (abbreviation: NaOtBu), 0.13 g (0.31 mmol) of 2-dicyclohexylphosphino-2′,6′-dimethoxybiphenyl (abbreviation: Sphos), and 40 mL of xylene were put in a 200 mL three-neck flask and the air of the flask was replaced with nitrogen and the mixture in the flask was stirred under a reduced pressure in the flask for degassing of the mixture. After the degassing, 0.14 g (0.15 mmol) of tris(dibenzylideneacetone)dipalladium(0) was added thereto, and the mixture was stirred at 140° C. under a nitrogen stream for 4 hours. After the predetermined time elapsed, the resulting reaction mixture was suction-filtered through a filter aid of Celite and alumina stacked in this order. The filtrate was concentrated to give a solid. The solid was purified by silica column chromatography. As a developing solvent, a 2:1 hexane-toluene mixed solvent was used. The obtained fraction was concentrated to give a target solid. Toluene was added to the solid, ethanol was added to the solution, and the solution was concentrated under reduced pressure, whereby an ethanol suspension was obtained. The precipitated solid was filtered to give 3.0 g of a target white solid in 70% yield. Then, 3.0 g of the solid was purified by a train sublimation method. The purification by sublimation was performed by heating at 350° C. under a pressure of 3.0 Pa with an argon flow rate of 10 mL/min for 18 hours. After the purification by sublimation, 2.2 g of the target white solid was obtained at a collection rate of 73%. A synthesis scheme of Step 3 is shown in (b-3) below.

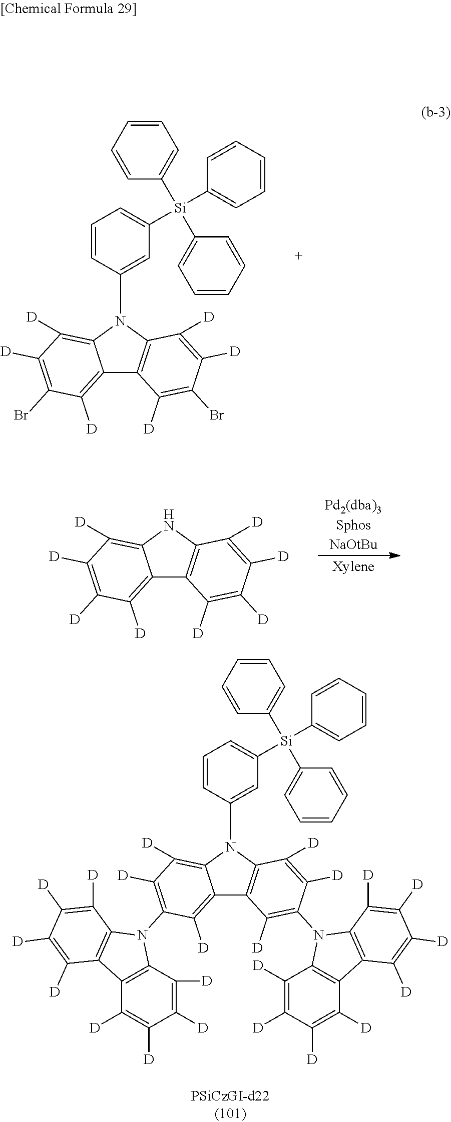

### <Measurement of Physical Properties>

Next, ultraviolet-visible absorption spectra (hereinafter simply referred to as “absorption spectra”) and emission spectra of PSiCzGI-d22 in a solid thin film were measured. The solid thin film was formed over a quartz substrate by a vacuum evaporation method.

The absorption spectrum was measured with a UV-visible spectrophotometer (U-4100, Hitachi High-Technologies Corporation). The absorption spectrum of the thin film was calculated using an absorbance (−log₁₀ [% T/(100−% R)]) obtained from the transmittance and reflectance of the thin film including the substrate. Note that % T represents transmittance and % R represents reflectance. The emission spectrum was measured with a fluorescence spectrophotometer (FP-8600, JASCO Corporation).

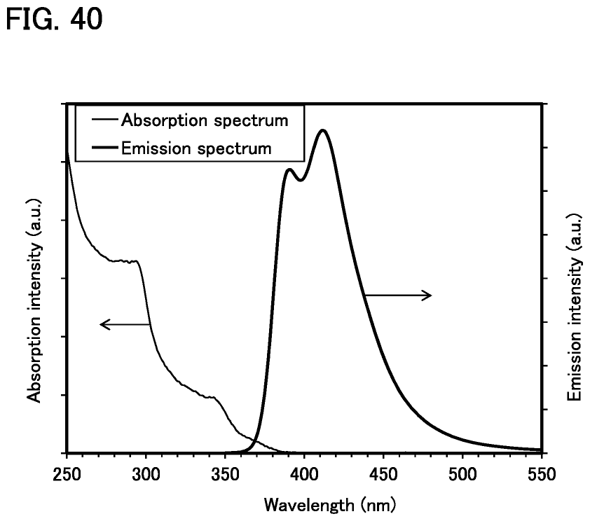

FIG. 40 shows the measurement results of the absorption and emission spectra of the obtained solid thin film. The horizontal axis represents wavelength, and the vertical axis represents absorption intensity and emission intensity.

As shown in FIG. 40, the thin solid film of PSiCzGI-d22 has absorption peaks at around 297 nm, 335 nm, 345 nm, and 357 nm, and emission wavelength peaks at around 391 nm and 412 nm (at an excitation wavelength of 330 nm).

### <Measurement Result of PL Spectrum of Mixed Film>

Here, PL spectra at room temperature and 10 K of the mixed film of SiTrzCz2 and PSiCzGI-d22 obtained in this synthesis example were measured. The measurement of the spectra was performed on thin films with a thickness of 50 nm deposited on the quartz substrate. The mixed film of SiTrzCz2 and PSiCzGI-d22 was deposited by co-evaporation of SiTrzCz2 and PSiCzGI-d22 at the weight ratio of 1:1 (SiTrzCz2:PSiCzGI-d22=1:1).

An ultraviolet-visible spectrophotometer, U-4100 (Hitachi High-Technologies Corporation) was used for measuring the absorption spectrum; a spectrofluorometer, FP-8600DS (JASCO Corporation) was used for measuring the fluorescent spectrum; and a microscope PL spectrometer, LabRAM HR-PL (HORIBA Scientific) was used for measuring the phosphorescent spectrum. In the measurement of the phosphorescent spectrum, irradiation of excitation light of 325 nm was performed for 0.1 sec and no detection or excitation was performed for 0.02 sec. Then, luminescence from the film at 10 K was detected for 0.86 sec and no detection or excitation was performed for 0.02 sec. These two steps were repeated to measure the phosphorescent spectrum.

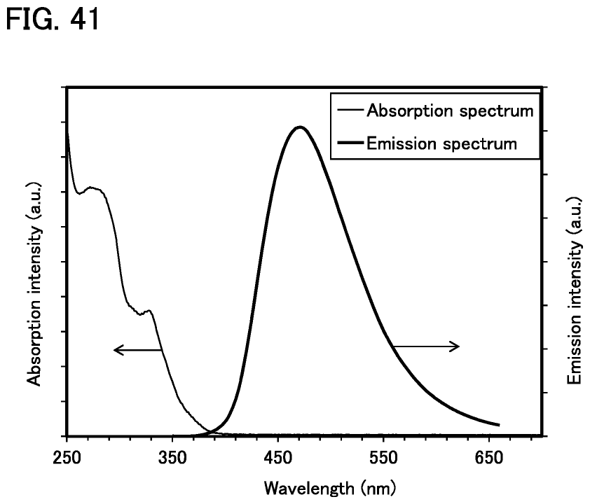

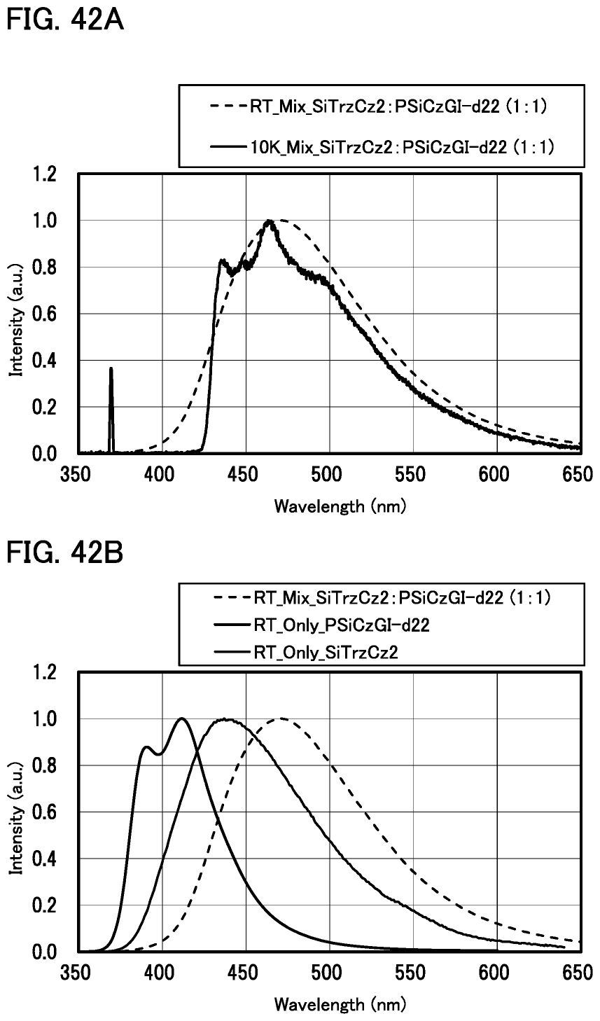

FIG. 41 shows measurement results of the absorption spectrum and the emission spectrum of the mixed film. FIG. 42A shows the measurement results at room temperature and 10 K of the emission spectrum of the mixed film. The horizontal axis represents wavelength, and the vertical axis represents absorption intensity and/or emission intensity. FIG. 42B shows the measurement results of PL spectra at room temperature of the mixed film of PSiCzGI-d22 and SiTrzCz2, the film of only PSiCzGI-d22, and the film of only SiTrzCz2. Note that explanatory notes are each shown in the order of the measurement temperature (room temperature or 10 K), the film state (a film of only a substance or a mixed film), and the organic compound in FIGS. 42A and 42B.

As shown in FIGS. 42A and 42B, PSiCzGI-d22 and SiTrzCz2 in the mixed film were excited at room temperature to form an exciplex and a fluorescent component of the emission spectrum originating from the exciplex is observed. On the other hand, a plurality of peaks were observed at 10 K, which indicates that a superimposed spectrum of the phosphorescent spectrum of PSiCzGI-d22 and the phosphorescent spectrum of SiTrzCz2 was observed.

This application is based on Japanese Patent Application Serial No. 2022-192372 filed with Japan Patent Office on Nov. 30, 2022, Japanese Patent Application Serial No. 2022-193138 filed with Japan Patent Office on Dec. 1, 2022, and Japanese Patent Application Serial No. 2023-003459 filed with Japan Patent Office on Jan. 13, 2023, the entire contents of which are hereby incorporated by reference.

What is claimed is:

1. A light-emitting device comprising:
a pair of electrodes; and
an organic compound layer between the pair of electrodes,
wherein the organic compound layer comprises a light-emitting layer,
wherein the light-emitting layer comprises a first organic compound, a second organic compound, and a light-emitting substance,
wherein the first organic compound comprises deuterium, and
wherein in a PL measurement of a mixed layer of the first organic compound and the second organic compound, a spectrum of an exciplex is observed at room temperature, and a plurality of peaks are observed at a temperature in a temperature range of 4K to 80 K, inclusive.

2. A light-emitting device comprising:
a pair of electrodes; and
an organic compound layer between the pair of electrodes,
wherein the organic compound layer comprises a light-emitting layer,
wherein the light-emitting layer comprises a first organic compound, a second organic compound, and a light-emitting substance,
wherein the first organic compound comprises deuterium, and
wherein in a PL measurement of a mixed layer of the first organic compound and the second organic compound, a spectrum of an exciplex is observed at room temperature, and a phosphorescent spectrum of the first organic compound and a phosphorescent spectrum of the second organic compound are observed at a temperature in a temperature range of 4 K to 80 K, inclusive.

3. The light-emitting device according to claim 1,
wherein the first organic compound comprises three or more carbazole skeletons.

4. The light-emitting device according to claim 1,
wherein the first organic compound comprises a deuterated carbazole skeleton.

5. The light-emitting device according to claim 1,
wherein in the PL measurement of the mixed layer of the first organic compound and the second organic compound, a spectrum of the exciplex is not observed at a temperature in the temperature range of 4 K to 80 K, inclusive.

6. The light-emitting device according to claim 1,
wherein a difference between a T₁ level of the first organic compound and a T₁ level of the second organic compound is less than or equal to 0.100 eV.

7. The light-emitting device according to claim 1,
wherein the light-emitting substance is a phosphorescent substance.

8. The light-emitting device according to claim 1,
wherein the light-emitting substance is a metal complex containing platinum.

9. The light-emitting device according to claim 2,
wherein the first organic compound comprises three or more carbazole skeletons.

10. The light-emitting device according to claim 2,
wherein the first organic compound comprises a deuterated carbazole skeleton.

11. The light-emitting device according to claim 2,
wherein in the PL measurement of the mixed layer of the first organic compound and the second organic compound, a spectrum of the exciplex is not observed at a temperature in the temperature range of 4 K to 80 K, inclusive.

12. The light-emitting device according to claim 2,
wherein a difference between a T₁ level of the first organic compound and a T₁ level of the second organic compound is less than or equal to 0.100 eV.

13. The light-emitting device according to claim 2,
wherein the light-emitting substance is a phosphorescent substance.

14. The light-emitting device according to claim 2,
wherein the light-emitting substance is a metal complex containing platinum.

15. An organic compound represented by General Formula (G1-1) or General Formula (G1-2), 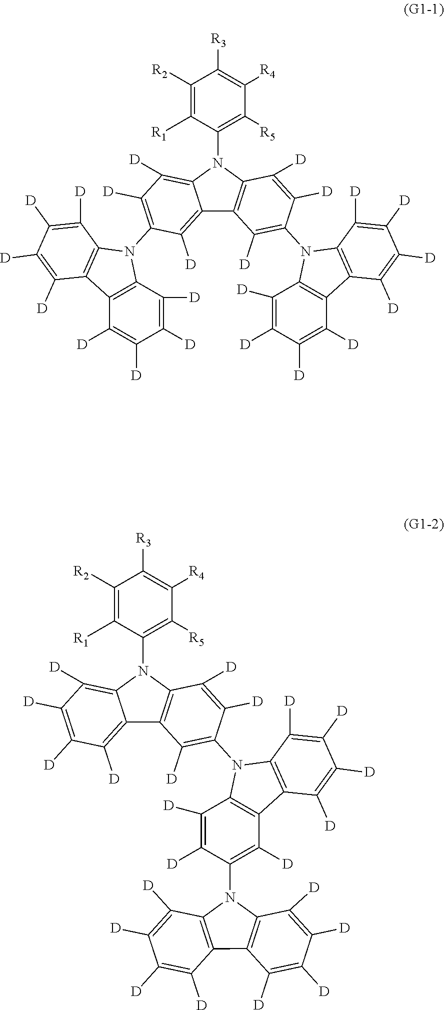
wherein R¹ to R⁵ each independently represent hydrogen, deuterium, a substituted or unsubstituted alkyl group having 1 to 10 carbon atoms, a substituted or unsubstituted cycloalkyl group having 3 to 10 carbon atoms, a substituted or unsubstituted polycyclic alkyl group having 6 to 10 carbon atoms, a substituted or unsubstituted aryl group having 6 to 30 carbon atoms, a substituted or unsubstituted heteroaryl group having 2 to 30 carbon atoms, a triarylsilyl group having 18 to 36 carbon atoms, or a trialkylsilyl group having 3 to 12 carbon atoms.

16. The organic compound according to claim 15,
wherein the General Formula (G1-1) is represented by Structural Formula (100) or Structural Formula (101),

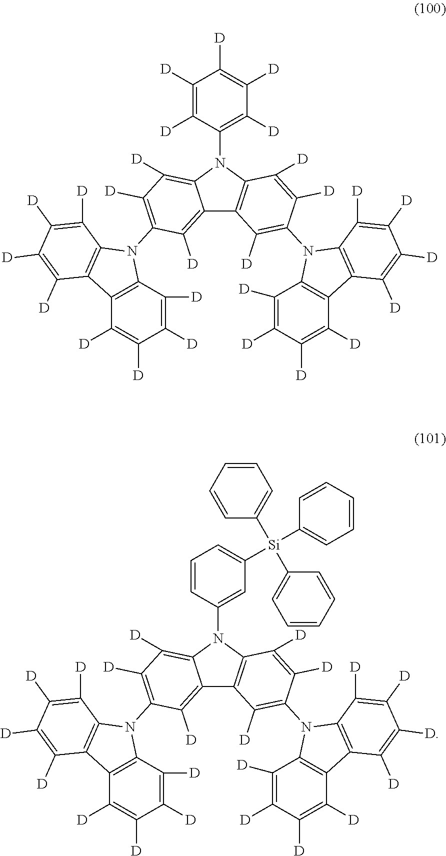

17. A light-emitting device comprising an organic compound layer between a pair or electrodes,
wherein the organic compound layer comprises a light-emitting layer, and
wherein the light-emitting layer comprises the organic compound according to claim 15.
